SEQUENCE LISTING (84 sequences)

SEQ ID NO: 1 - DNA, 21 bp, Arabidopsis thaliana
  source 1..21: ecotype = Columbia-0; mol_type = other DNA
ttgactgatgggatgtccttc

SEQ ID NO: 2 - DNA, 21 bp, Arabidopsis thaliana
  source 1..21: ecotype = Columbia-0; mol_type = other DNA
tggagatttgaagcataacgg

SEQ ID NO: 3 - DNA, 21 bp, Arabidopsis thaliana
  source 1..21: ecotype = Columbia-0; mol_type = other DNA
atttgatggtgcaggattgtc

SEQ ID NO: 4 - DNA, 21 bp, Arabidopsis thaliana
  source 1..21: ecotype = Columbia-0; mol_type = other DNA
tgatggttttgcattgttgag

SEQ ID NO: 5 - DNA, 21 bp, Arabidopsis thaliana
  source 1..21: ecotype = Columbia-0; mol_type = other DNA
gcgtgcttgagttttcaatgt

SEQ ID NO: 6 - DNA, 20 bp, Arabidopsis thaliana
  source 1..20: ecotype = Columbia-0; mol_type = other DNA
cggtatgcaagctgaggttc

SEQ ID NO: 7 - DNA, 20 bp, Arabidopsis thaliana
  source 1..20: ecotype = Columbia-0; mol_type = other DNA
agaacctgcgaaggagtgtg

SEQ ID NO: 8 - DNA, 20 bp, Arabidopsis thaliana
  source 1..20: ecotype = Columbia-0; mol_type = other DNA
ggaggattctacagccacgg

SEQ ID NO: 9 - DNA, 20 bp, Arabidopsis thaliana
  source 1..20: ecotype = Columbia-0; mol_type = other DNA
ccgttccctataccaggagg

SEQ ID NO: 10 - DNA, 20 bp, Arabidopsis thaliana
  source 1..20: ecotype = Columbia-0; mol_type = other DNA
tcgctatcgctttggttctc

SEQ ID NO: 11 - DNA, 20 bp, Arabidopsis thaliana
  source 1..20: ecotype = Columbia-0; mol_type = other DNA
tccaccagcaataacctggg

SEQ ID NO: 12 - DNA, 20 bp, Arabidopsis thaliana
  source 1..20: ecotype = Columbia-0; mol_type = other DNA
tgccttagctgttgccattg

SEQ ID NO: 13 - DNA, 20 bp, Arabidopsis thaliana
  source 1..20: ecotype = Columbia-0; mol_type = other DNA
atttgattcagcagctcggc

SEQ ID NO: 14 - DNA, 20 bp, Arabidopsis thaliana
  source 1..20: ecotype = Columbia-0; mol_type = other DNA
tatgatgcccagggggtgag

SEQ ID NO: 15 - DNA, 20 bp, Arabidopsis thaliana
  source 1..20: ecotype = Columbia-0; mol_type = other DNA
gcccctgctatgacttcaac

SEQ ID NO: 16 - DNA, 2340 bp, Arabidopsis thaliana
  source 1..2340: ecotype = Columbia-0; mol_type = genomic DNA
gcgaccgttgaatttttctcagaaaataaaaatatataagctaaagattcataatacccaaagtacccctattgaaaagtcaaaccttgtagcaagcccaaaggcccactttatttgagcccagttatcctcttttggacacatttttggtatattcgagtcacgatactcgaatggtatttccgttataatttacgaaaacaatgtctcttcttttctctacaaccacacgatttcgatgttaattaccaaaaaagtgtgtttaatttagctaaactaattgttttcgttgattaggcccagttgggttgaagaaaatctccaatctttcttcgccaagtctcccacaacactttgtgttctaatctctctgtatgtcgcaaagtagcagcaacttaattctctcatggagtctcttacatcaaatccttccttcgcttgtttctctaaaaccatttctcattacccttctcgtttctccttcgtaaagctaacctctatccaaaagttggaagcttccaacaacaccttatcgttattctgctgtaaatcatcttcgaatcctaaaccagattgcaatcgtcgtatcaagttaaaccctttatgtgtattgcgtccaatcattcgaacaataaagggtttggtttcttctcaatccaggcaatggatgtctcgttttcgagcttacagagacgacacggcggcgttttctgaggatttcgctggagatttgaagcataacggtggattagggattgcccttttgagcgttactgcttctgctaagataaagattagccctttcgttgcgacgctctctgcgaatccaactttcgtttcagctgtagtcgcgtggttctttgcgcaatcgagtaaaatggttatcaatttcttcattgagaggaaatgggattttcgtcttctctatgcttctggtgggatgccttcgtctcactcagctttatgtatggctttgacgacctctgtggcgctttgtcatggagttgcagattccttgtttcctgtttgtttagggtttagcttgattgtcatgtatgatgcaattggtgttagacgccatgccggtatgcaagctgaggtaaaaagttaaaccaaatctgcttcaagttttgagttttggagatttttacagagtttgactttggtcttattaaatgctttgtctattctacaagttgactagattttcatgttgatgagtgtgtatagtttaatgtattactatttccatgttgatgagtgtgtatagtttaatgtattactagaatctggtgatggattcgactttcactacattgttgaatttttgtgaatgcttttatgtgtaaaagttggcgtgcttgagttttcaatgtgtgcacaattgaagtacatcactagatttggaattatggagagttagatgttgatttctgttcttggttcttgaatcttttatatgatggttttgcattgttgagagcgtaatacaagtttctcgaaattatggacattgggcacatgttgatgatagctgttattggttgtagaatgaccttgataaaattgcagagaattaagagttttgaaagaggattcacacagagtctaagtcatgtttgctcttggttcttaaatcttttagatgatggttttgcattgttgagagcacaatacaagtttctcgaaattatggatattagccacatattgatgatagttgttgttggttgtagaatgatcttgataaaattgcagagaattaagagttttgaaagagggtctgtacagagtctaagtctagtttgttgttctttgtaagataagattaacaatgttttgtatttgttgatgtcaggttctgaacttgatcattagggacttgttcgaaggacatcccatcagtcaaagaaagcttaaggaactgcttggtcacactccttcgcaggttcttgctggagcattggttggtattgtgattgcttgcttttgttgccaaggctatctcgtctcagtctaagaagatgtcttttattccaagtgatctggttggtttttgatttgggtagcctaaaaaaacagagtgttttaagtatcgcttaacattgattttggctaagattcatgttgtagtatcatatgttttgttcagcaatagagaaaactcttgtttcttcttccttgtggcttttatttttttgagtttttgtattccctgagaatgcaatgagagcttcttttgtttaaagcgtttaaagcaatgcatgacatgttcagtgactgctgcagaaacacacacacacacacacacacaaagttaaccataataactaagattta

SEQ ID NO: 17 - DNA, 1955 bp, Arabidopsis thaliana
  source 1..1955: ecotype = Columbia-0; mol_type = genomic DNA
atgaacatcacaattttattccacaaattcctttgtatactacttattttcaaataaaatcacgttatttttgtaatggtgtttcacgatttctttacatatggtaatggtaatgttaaacaagaataaatatatttttaactcggattcggttgtaaatctgttttggtactctccagtggcttcaataggcaatgaagcagaagagttgtgtctatgcatgttcttgacgttagcttgtagctatcctgaaaaataagtatccggtgactgctgtagcacttccaagcattccaccagcaacgacctgaattctcagcaaatggaaaacagaatctgatgagaactagaaattcattgatatcaacacggttacaaatcaaagagtagttcatttatcaaccaaagtaaacacaaactagggtctgttctagagttccatcagctcacacaagcctccaaaagaccctcaactctgttcccaattacacgatccccctcaccgattccttcagcactctatttgtactcatgatttcccctaactctgtaactatcttgctaaatgtaacagctaaaagaatctcatatcttgctagtcatatatgaaaagactttttcaagtcatatgtctacatacctggggaggggtatgaccaagaagttcccgcaaaggtctgctttcagccagcggatgttctgcaggaagttcatatacaatctgattgagaacctgaaaaatgaatcagaccccagagatcaggataccagagctcaaatgcatcatgacaaaaccaatagtggttcttttatcaaaccagtcaagttagaagatgaaacaaaggaaaggttagagcagcaatgaacaaacctcagcttgacgaccagcatgtaatctaacaccggtagcatcatacatcacctaccaacccaaaccactcacatattaatcgtcacttgattccttaatagccaatttgattaaactagagaaagtatccaacttacaacagatgccaaaatcaaagcaatggcaaaatgtgatccaccaaagccttcttgtaaaccaatagctacagcgagagctgtaacggtcgctgaatgcgaagaaggcattcctccagacccaataagctgtttgagatcccatctccgttccctatacctacagaatctaggaaaccatgtaaagatcctttctcatctaacgtatactataatttcctatgtaccgtttcgaaccaaagattgaggaaccaggcacatatagatttaatcggagtctgatccaaacagagatataaagagagaacgtaccaggaggtgaagagtttgatgaattgagcgatagtgaaagaggtgacggcggaaatgagaggataattcgtgaaaatggagaaataatgagtggaagacgaagacgtagccgtggctgtagaatcctccatggccgatgatggatctagtcaggctttaaatcgtgctagcgaaagaagaagaagaaaatgacaccgaaacgacaatgatccataaaacgtcgactaattccctttcaaactccaatggcgtctacgtctaccgttcaatcctctttgtctttctttttttttctttgaccaattttttttttaaacactgctccacaaaactatatattatacaattacccgcatccgaacaaatatgaaccgtatccgatacaaaacccgaatacctaggtcagttctattcgtgaaatcgagtctcctcttattcgacgacctgccccaactataaacatgacttttacacacttgacataacttcgtttgtatcgcttttttgaaattctatatattattattgtttgagcagaaactaagacaaaattaagacttttctctcttcttcttctccactgggtcgatctttacgaggttcacttgtacggattcactttaacaacagattcgcca

SEQ ID NO: 18 - DNA, 1848 bp, Arabidopsis thaliana
  source 1..1848: ecotype = Columbia-0; mol_type = genomic DNA
tatcacacattagaggaattttatcagctagcttgtgccagaggaaacagaacttaatgctaatatatatgtgtttaatgagccaaaagaaataaagcaaatcaacattagctctataagcaaaactttgtagtcatggaatttaagaggaaatgagatgatgagttttaaagtattgatgctcctgagtgctgaatctaaacagatcatgttcgttgtagttcaatgcacaagctcccattttaccactttacagatctgtgtaatgacgtttcaagtttcctttactagagcaaacctaactaaaatatattgtcaatgacaagaacagagcagcaaaaaaaacagagaaattaagctcaatttccaaattctatttgcatgtcatggctatctttaaactacctagcagcgccttgacacacaatggagcagaaaaggcgagattttgctgttcccagcttaagctacttggctaacaagatgactaagtacccaacaactgctgtagatattccaagcattccaccagcaataacctgacgccaccaaaatatagatagtctcaagtactaaccacaatcattctcttgcttggtgtgatctgacacaattagtactacatacttacctggggaggagtatgacctagaagttcacgcaaaggtctggtttcagccagaggatgctctgcaggaagttcatacacaatctgatttagaacctcagcttggcgtccagcatgtaaccttacaccagttgcatcatacataacctaccaaaccaaacaatcttaaattcttaatcatcccctgaagtgatataatcaagaaaggtcattaattaattaagtgattagctaaaaacatacaatggtagtgagaaccaaagcgatagcgaaatgtgaaccaccaaaaccttcttgtaaaccaacagctaaagctaaagctgtaaccgttgctgaatgtgatgaaggcatacctccagacccaacaagccgtttcaaatcccatctcctttctttatacctacaccaagtagttgtaaaatcaaatttctacaactagtctgtttctttaacctagacagatatgaaaacgtaccaggaggtgaagaatttgatgaattgtgcgatggtgaaagctaaaacggcggaaatcagagggtaatttgtgaaaattgagatatagtgcgaggaagatgaagccaccgattcctccattgaatgatggtgatcgattgatctcgtatcgtagggctttaatcgtctgatagctcttctacaggataaagaacatacagagaagacataaagttgcgtgtagaaatctttaccttgagactcctctaatggcttcttcttcctcaaattttattttcctagttttctcgctttttcttcggcaacaaacaacagcttttatttaatgttttttactaacaagtctacacgacctaagtacagatgagattattacttttttttatcaccgtttatattataatgacaaaacagctacatatcaataaagaattgacatctgaaaaattgcatattatctgaaattatataatagaaatattattttataaactctgttgatatgttagaagtttttgcgatattaatatacgtatagagctttttattgtttaaagtttttatccagaaagaaagtttaaattctgctagttggttcaaaaatgcacattcacaaaaatatagcatgcgatattagaatactgaatacactatcaatctatgattgatcagattatcagaatatgcttctgagcttctctgtttgctagagaatatgctttttaggctttaagtgaaaaacaact

SEQ ID NO: 19 - DNA, 2018 bp, Arabidopsis thaliana
  source 1..2018: ecotype = Columbia-0; mol_type = genomic DNA
tctgattctacaattgccatgccagtcattaacgaataatatccaatacagatcttcgaattagttaagctgactcaaaagtttcactagcttcacattttctatcaataatggtatcaataatgtgcttttaggccataaaacttcttgtagtctagtggtcatagatatggtttcgcataaaactgatcatgattaggaggaagaggaaatagagagattatatatatatatatagagagagagagaatgttgagaaggcgtaagcttaaacaatgacaagtctaactaagatcatctatatattacacagatttaacaatttccatttactccaataatctagcgtcacttgatatcgctgaatctatcttagtacctgaagggtctatgatttcaaaagaattcgggatttgttcttgtgtagagagactgactagcttgagctcctcattaagtaagctaccacgcatcctaaaatcccacctgctgcaacctgtatttcaatctcataacttagcaatagaatgtaacatcctatggttaatacatggcgaatacggtgactttagttctatatgtttcagctttatgaaatctagatctaattctaacatggttatgacacagtcattggacttaaaaatgtttaatgattggaatgataaaaagaattgactggtgggtatagaaaacctggataggagtgtggccaagcagttcacgcaatggtctaactgttgataatggatgctccggaggaaactcacaaacaatttgattcagcagctgaaatttccaatcaagtgaaaaaggcttgcggtttcagaaagcatggaacaataaaagaagatgaaaacagatgagattaataacttttctatagctttacctcggcttgacgaccagcatgaagcctgacaccagaggcatcatacataacctgcagcccaattcagaaatatacaaaagattggtcttactatgaaaccaaataggctattcacaatgaaagcaaaaatctcttttctacactaaacaaagcaaggcatttgtaagacaaattgtgaagtggaaaagaagatgagaagaacactcacaacgcaagccaagacaactgcaatagcaaaagctggtgctccagctccttcttcaaagccaatggcaacagctaaggcagtgacagtagcagaatgagaagaaggcattccaccagaactaatcatacgtttagagtcccacctcttttctttgtacctgaacatctaacatacatcacgaaacaactgagaattccacttctttcaatcttaactacaacagatctatagctacacaaatgccaacattcaacttctgagacctaatcatgagcaaaaatttgcaaatcctaattaatccatcgaaaatcgaaaccctaacagtaagaaatttgttcaatcacagatcttaagcagagatcaaactaatctgagaaaacgaaagatttgaacagaccaattagtgaagactttgaggaactgagcaagagcgaaggcaagaaaagcggagaagattgggagattgtgaggaaacagattatgcgacggaggagatccgtacaacgccctattgccgccacctaacgaaccaacgtccgccgctgtcatcacctcgtccataggcgatatacaaccgcgaaaaaatcccgatctcttcttcttcttcgatcaaaatcagacactgcgaggatcaatgtcaacgtaaaaaggtaaagagattcaggagaagagagtattgaattgttttcgataagcttttaggctttttgtgttggttccgtatttgggaaggatcaattattgtgttaaaaggaaatcaattttacctttttaatttttttgcacttttgtttttcaatattgctgaatgttgactaataacttatttgatttgttttagataaatttgatgacctctatttgatattgttttttttttgtatatatatgatatagcaaaaaccaaagtaaactttg

SEQ ID NO: 20 - DNA, 1415 bp, Arabidopsis thaliana
  source 1..1415: ecotype = Columbia-0; mol_type = genomic DNA
taaattctctttctttcttatcaacacaaaaaatatatagtttttataaagttgggagcttttttacatcagggaataaacactgagggtaactaagaaaccgaacaaagcccctgctatgacttcaacttcagtgtgtcctattgattctttcaatggaggagcaatctcctcactgatctcttctgatgtcaaagctttgttagattcgttgccctttaaactcattacttcacttctacgagcattagctgttagtttgttcaacactttggcatgttttccaacttctctcctcaccccctgcaatgttacaatcagtgagacattaagataacactagaatcagacattgagaatttgagaataaccaacaaagacctattgaacaaaagccagcctaagcttttctctatagactattttacctgggcatcatacatgataaggccagcataaaccacagttagaccaaaaatggagtcagcaaatcccctgaagccaaaccatattcaggaaattgcataagctgagtatatttaggggaattgagaagagaaaggttcaggctaatcacctttcaaaagcaatagcagttgctgcagccacaacagactggtttctcaagaattaggccaaaatgtcagagaagtagacaaacaatattaccagaggaaacaagaataaaaaaaggataagcttacagaagaatgtgttgaagggaaacctccagcttgaaagacacttctaaagtccagatttttcccatacaaaacgacagaagtgaatggctttgagagctgcccaatcacagcagaagtcccagcagctatgagtaccttcacgccggtgaaatcaaaagcctaagtgacgatgaccctccaaaaaaaggaatgaaagaagagaaaaaggcgaaaccttgttgtggatgacctcagcgatatcttggaatccaacattgacaacgcaggtgagacgaggtggcttctttgggcagcttgcaagattgaatttggagttggagaaagagaaacagagatcattggcgttatgagacacaaaacagtgcttcatcaacatctctgtgcagaagatggaactttggtcggaggatgacgataagaataaggcagcgagaaatttggaaccaatggttctgacgctctgttatgtcgagaagagtgttgtctcttaggacattttaattgactcagagaagcttttgtggattttttcattgacaaattgttatctcacacacaaaatctgctttaactcttttagtaaaactcttttagtctcttaaaccaagtcatctgttcttcttttataaatcacaatttgtattcgccaaaattaaagaatatgaacaaaaacaaacatatcgaaactttcagcttgtggagttttaagtc

SEQ ID NO: 21 - DNA, 855 bp, Arabidopsis thaliana
  source 1..855: ecotype = Columbia-0; mol_type = genomic DNA
atggagtctcttacatcaaatccttccttcgcttgtttctctaaaaccatttctcattacccttctcgtttctccttcgtaaagctaacctctatccaaaagttggaagcttccaacaacaccttatcgttattctgctgtaaatcatcttcgaatcctaaaccagattgcaatcgtcgtatcaagttaaaccctttatgtgtattgcgtccaatcattcgaacaataaagggtttggtttcttctcaatccaggcaatggatgtctcgttttcgagcttacagagacgacacggcggcgttttctgaggatttcgctggagatttgaagcataacggtggattagggattgcccttttgagcgttactgcttctgctaagataaagattagccctttcgttgcgacgctctctgcgaatccaactttcgtttcagctgtagtcgcgtggttctttgcgcaatcgagtaaaatggttatcaatttcttcattgagaggaaatgggattttcgtcttctctatgcttctggtgggatgccttcgtctcactcagctttatgtatggctttgacgacctctgtggcgctttgtcatggagttgcagattccttgtttcctgtttgtttagggtttagcttgattgtcatgtatgatgcaattggtgttagacgccatgccggtatgcaagctgaggttctgaacttgatcattagggacttgttcgaaggacatcccatcagtcaaagaaagcttaaggaactgcttggtcacactccttcgcaggttcttgctggagcattggttggtattgtgattgcttgcttttgttgccaaggctatctcgtctcagtctaa

SEQ ID NO: 22 - DNA, 507 bp, Arabidopsis thaliana
  source 1..507: ecotype = Columbia-0; mol_type = genomic DNA
atggaggattctacagccacggctacgtcttcgtcttccactcattatttctccattttcacgaattatcctctcatttccgccgtcacctctttcactatcgctcaattcatcaaactcttcacctcctggtatagggaacggagatgggatctcaaacagcttattgggtctggaggaatgccttcttcgcattcagcgaccgttacagctctcgctgtagctattggtttacaagaaggctttggtggatcacattttgccattgctttgattttggcatctgttgtgatgtatgatgctaccggtgttagattacatgctggtcgtcaagctgaggttctcaatcagattgtatatgaacttcctgcagaacatccgctggctgaaagcagacctttgcgggaacttcttggtcatacccctccccaggtcgttgctggtggaatgcttggaagtgctacagcagtcaccggatacttatttttcaggatagctacaagctaa

SEQ ID NO: 23 - DNA, 492 bp, Arabidopsis thaliana
  source 1..492: ecotype = Columbia-0; mol_type = genomic DNA
atggaggaatcggtggcttcatcttcctcgcactatatctcaattttcacaaattaccctctgatttccgccgttttagctttcaccatcgcacaattcatcaaattcttcacctcctggtataaagaaaggagatgggatttgaaacggcttgttgggtctggaggtatgccttcatcacattcagcaacggttacagctttagctttagctgttggtttacaagaaggttttggtggttcacatttcgctatcgctttggttctcactaccattgttatgtatgatgcaactggtgtaaggttacatgctggacgccaagctgaggttctaaatcagattgtgtatgaacttcctgcagagcatcctctggctgaaaccagacctttgcgtgaacttctaggtcatactcctccccaggttattgctggtggaatgcttggaatatctacagcagttgttgggtacttagtcatcttgttagccaagtag

SEQ ID NO: 24 - DNA, 525 bp, Arabidopsis thaliana
  source 1..525: ecotype = Columbia-0; mol_type = genomic DNA
atggacgaggtgatgacagcggcggacgttggttcgttaggtggcggcaatagggcgttgtacggatctcctccgtcgcataatctgtttcctcacaatctcccaatcttctccgcttttcttgccttcgctcttgctcagttcctcaaagtcttcactaattggtacaaagaaaagaggtgggactctaaacgtatgattagttctggtggaatgccttcttctcattctgctactgtcactgccttagctgttgccattggctttgaagaaggagctggagcaccagcttttgctattgcagttgtcttggcttgcgttgttatgtatgatgcctctggtgtcaggcttcatgctggtcgtcaagccgagctgctgaatcaaattgtttgtgagtttcctccggagcatccattatcaacagttagaccattgcgtgaactgcttggccacactcctatccaggttgcagcaggtgggattttaggatgcgtggtagcttacttaatgaggagctcaagctag

SEQ ID NO: 25 - DNA, 639 bp, Arabidopsis thaliana
  source 1..639: ecotype = Columbia-0; mol_type = genomic DNA
atgttgatgaagcactgttttgtgtctcataacgccaatgatctctgtttctctttctccaactccaaattcaatcttgcaagctgcccaaagaagccacctcgtctcacctgcgttgtcaatgttggattccaagatatcgctgaggtcatccacaacaaggtactcatagctgctgggacttctgctgtgattgggcagctctcaaagccattcacttctgtcgttttgtatgggaaaaatctggactttagaagtgtctttcaagctggaggtttcccttcaacacattcttcttctgttgtggctgcagcaactgctattgcttttgaaaggggatttgctgactccatttttggtctaactgtggtttatgctggccttatcatgtatgatgcccagggggtgaggagagaagttggaaaacatgccaaagtgttgaacaaactaacagctaatgctcgtagaagtgaagtaatgagtttaaagggcaacgaatctaacaaagctttgacatcagaagagatcagtgaggagattgctcctccattgaaagaatcaataggacacactgaagttgaagtcatagcaggggctttgttcggtttcttagttaccctcagtgtttattccctgatg

SEQ ID NO: 26 - protein, 284 aa, Arabidopsis thaliana
  source 1..284: note = Columbia-0 ecotype
MESLTSNPSFACFSKTISHYPSRFSFVKLTSIQKLEASNNTLSLFCCKSSSNPKPDCNRRIKLNPLCVLRPIIRTIKGLVSSQSRQWMSRFRAYRDDTAAFSEDFAGDLKHNGGLGIALLSVTASAKIKISPFVATLSANPTFVSAVVAWFFAQSSKMVINFFIERKWDFRLLYASGGMPSSHSALCMALTTSVALCHGVADSLFPVCLGFSLIVMYDAIGVRRHAGMQAEVLNLIIRDLFEGHPISQRKLKELLGHTPSQVLAGALVGIVIACFCCQGYLVSV

SEQ ID NO: 27 - protein, 168 aa, Arabidopsis thaliana
  source 1..168: note = Columbia-0 ecotype
MEDSTATATSSSSTHYFSIFTNYPLISAVTSFTIAQFIKLFTSWYRERRWDLKQLIGSGGMPSSHSATVTALAVAIGLQEGFGGSHFAIALILASVVMYDATGVRLHAGRQAEVLNQIVYELPAEHPLAESRPLRELLGHTPPQVVAGGMLGSATAVTGYLFFRIATS

SEQ ID NO: 28 - protein, 163 aa, Arabidopsis thaliana
  source 1..163: note = Columbia-0 ecotype
MEESVASSSSHYISIFTNYPLISAVLAFTIAQFIKFFTSWYKERRWDLKRLVGSGGMPSSHSATVTALALAVGLQEGFGGSHFAIALVLTTIVMYDATGVRLHAGRQAEVLNQIVYELPAEHPLAETRPLRELLGHTPPQVIAGGMLGISTAVVGYLVILLAK

SEQ ID NO: 29 - protein, 174 aa, Arabidopsis thaliana
  source 1..174: note = Columbia-0 ecotype
MDEVMTAADVGSLGGGNRALYGSPPSHNLFPHNLPIFSAFLAFALAQFLKVFTNWYKEKRWDSKRMISSGGMPSSHSATVTALAVAIGFEEGAGAPAFAIAVVLACVVMYDASGVRLHAGRQAELLNQIVCEFPPEHPLSTVRPLRELLGHTPIQVAAGGILGCVVAYLMRSSS

SEQ ID NO: 30 - protein, 213 aa, Arabidopsis thaliana
  source 1..213: note = Columbia-0 ecotype
MLMKHCFVSHNANDLCFSFSNSKFNLASCPKKPPRLTCVVNVGFQDIAEVIHNKVLIAAGTSAVIGQLSKPFTSVVLYGKNLDFRSVFQAGGFPSTHSSSVVAAATAIAFERGFADSIFGLTVVYAGLIMYDAQGVRREVGKHAKVLNKLTANARRSEVMSLKGNESNKALTSEEISEEIAPPLKESIGHTEVEVIAGALFGFLVTLSVYSLM

SEQ ID NO: 31 - protein, 181 aa, Amborella trichopoda
MDEVLVAGDGRPFFFNNNNNNTTTPSSSSSPVSLSNLPLLSSFLAFAIAQFLKLFTTRYKEKRWDAKRLLGSGGMPSSHAATVTALAIAIGLREGAGSSMFALAVVVASVVMYDASGVRLHAGRQAELLNQIVCELPPEHPLSTVRPLREPLGHTPVQVVVGAVLGCMVAYLMRETPNNIN

SEQ ID NO: 32 - protein, 271 aa, Amborella trichopoda
MDFPSLSPFCVLIPVLHRITDYFTVKGFHGGKTLRTTMVKWVSGVKNGGRTGACLDDDLLNSGGIGMALLSTTAMAKGHISPVMATLSANPTFMSGLFAWVIAQSTKVLLNFFVERKWDFQMLVSSGGMPSSHSALCTALTTSVALCHGISDALFPVCLGFSLIVMYDATGVRRHAGMQAEVLNMIIEDLFQGHPVSQRKLKELLGHTPSQVIAGAFLGILVACMCCQGRIRVAMGLVARHGLSPNVKTAGSVQGGLTASDCRLCLTAPYG

SEQ ID NO: 33 - protein, 261 aa, Amborella trichopoda
MLSSTANFSSILAPKVLSSTAHKRPFVCLKFGIEEIKEISQNKVLVSATVAAAIGQFSKPFTSAIYGKGFDFAAAARSGGMPSTHSAGVVAAAAAIGLERGFSDSIFGLAVVLAAIVVYDSQGVRKEVGTHAKILNNTILKSEAQHGFLETENGDLADEDLEAASVEAGATISSLFSSTNDFYTKDEKRAKLNSNYSQINKPSDVLRKSLKMPLIEEAGKEAKKIGNRYPRLKESVGHTEAEVFVGILLGFLVTWAVYSVM

SEQ ID NO: 34 - protein, 105 aa, Amborella trichopoda
MPSSHSATVTALAVAIGFKDGFEGSVFAVAMVFACVVMYDAFGVRLHAGRQAEVLNQIVYELPAEHPLADTRPLRELLGHTPTQVIAGAMLGCVTAIVARILSLL

SEQ ID NO: 35 - protein, 199 aa, Chlamydomonas reinhardtii
MRPVFLEGPGTEVQYPVSAHTANYSDALTAREVILHTGFVGLFFNGCLVSAFIAFFIAQTCKVFTHYYTEQVWDLQRMVGSGGMPSSHTALIVALTTAVGVENGTSSTLFAACLVLALIVMYDATGVRLHAGRQATVLNIIIAEMPPDHPVQDGGRLRDSLGHTPIQVAVGAVLGVVVGLVVENLYLLGDKSGAGGSFH

SEQ ID NO: 36 - protein, 186 aa, Chlamydomonas reinhardtii
MRRQFDSAILADNSSMAQPGLAVPLLAAGLAIPPVPGNGVADLLTNRVFLVGFWSWFSAQFLKIFTKRFKKGVWDLGAMLESGGMPSSHSSLCAGITTAIAIQQGLGSPLFAACLCFSVIVMYDAMGVRRHAGKQAEVLNKVIDELLDDDHPMGEVKLKEVLGHTPRQVVCGGLLGLAVGLFFPAC

SEQ ID NO: 37 - protein, 279 aa, Medicago truncatula
MMLLQQSNFCPIPSSQFPSLKQRNPFLHHLQFRRKDKASTFRISSLAAAGFFNDVAQIAHNKVLIAAGVSMAIGQLSKPFTSVFLYGKEFDIKALIQAGGFPSSHSSATVACATLLGLERGLSDPIFGLAVVYAGLIMYDAQGVRREVGIHARTINKLLLQMHVNHLHSKHKDGLINSQPGSSSPPKAETQEKSLLFQETTSLEPQQANTNVLVKSESIIRQSDEELQSSDFLEDAKETSKLVADGLLPLKESVGHTEVEVVAGGLLGFLVGLAVYNLK

SEQ ID NO: 38 - protein, 167 aa, Medicago truncatula
MNEVLTRADVTASTASSLSPFVPSSNLPLISAFLSFALAQFLKIFTTWYKEKRWDSKRLLDSGGMPSSHSATVSALAVAIGFQEGIGSSVFAIAVILACIVMYDATGVRLHAGRQAELLNQIVCELPPEHPLSNVRPLRDSLGHTPLQVVAGGLLGCIIAFLMRKSS

SEQ ID NO: 39 - protein, 162 aa, Medicago truncatula
MNETAPTTSSSSSIFHNYPLISAILAFTIAQSIKFFTVWYKEKRWDPKQLVGSGGMPSSHSATVTALATAVGFHEGFGGPLFATALVMAIIVMYDATGVRLQAGRQAEVLNQIVIELPAEHPLSDSRPLRELLGHTPPQVIAGSLLGFITSSIGYIITMFGS

SEQ ID NO: 40 - protein, 254 aa, Medicago truncatula
MEYSLLPLTPKLPSSSFHFNTLRPISTSCSQSNSNNTTSTNSYNSQRNPTWVSFPFTQTIKHLTALNTHNKVSNENCCYLQNGGLGIALLSVTANAKVKISPFVATLASNPTFVSGLFAWFLAQSIKFFLNFFVERKWDLSLFCASGGMPSSHSALCTALTTSVAICHGVADSLFPVSLGFSLIVMYDAIGVRRHAGMQAQVLNMILADMFQGHPISERKLKELLGHTPSQVFAGALLGFLVACFCCQGCVVVG

SEQ ID NO: 41 - protein, 171 aa, Oryza sativa
  source 1..171: note = ssp. japonica
MGDDGGGDGGASSAGFSYFAVFHNYPLVAALLGFAVAQSIKFFVTRYKENRWDPKQLIGSGGMPSSHSATVTALAVAIGFQDGFGCALFATAAIFASVVMYDASGIRLHAGKQAEVLNQIVCELPSEHPLSETRPLRELLGHTPTQVVAGALLGSMLATAGQMFLVVSGSV

SEQ ID NO: 42 - protein, 153 aa, Oryza sativa
  source 1..153: note = ssp. japonica
MAAAAAVVNYPLVAALVAFALAQSSKFFTTWFKEKRWDARQLIASGGMPSSHSATVTALAVAIGIQEGYRSATFATSVIIACVVMHDAFGVRLHAGKQAEVLNQIVYELPEEHPLSETKPLREILGHTVPQVVAGCIIGILIAVVMRLALWSS

SEQ ID NO: 43 - protein, 231 aa, Oryza sativa
  source 1..231: note = ssp. japonica
MEVLSSSRFSRVFPSSLLSPVSKPLKPPRHRRRRVQTLSSSSSDAAAAPSTPPPVWPPASLSRLLAAALRGGRAGGELPDLAVGAGAAATAAGGGARIGTLLMSTTAAAVTKARENPYILALAANPTFVSGLVAWAVAQAAKVVLTSFVERRWDLRMLFSSGGMPSSHTALCTALTASVALCHGVSDSLFPVCLGFTLIVMYDATGVRRHAGMQAEVSQCFDCFCPFLYCI

SEQ ID NO: 44 - protein, 75 aa, Oryza sativa
  source 1..75: note = ssp. japonica
DYIFFLQVMYDASGIRFHTGRQAALLNQIVSDFPPEHPIISSFRPLQEPLGHSPFQVFAGALVGCSIAYLMGKSV

SEQ ID NO: 45 - protein, 171 aa, Oryza sativa
  source 1..171: note = ssp. japonica
AVVAVATSLGLERGFADSIFGMSVVFAAIVMYDAQGVRREVGNHARVLNKLLTLREKITQNPDDNSLLSSTSELHSSKPETVAELVSVAEKLGSSQGSSANPFPIHSSGTKSSRLNALQSSETEVTEFTQLKEAYTEECDRLSESVGHTELQVAAGALLGFLVTLVVYATL

SEQ ID NO: 46 - protein, 184 aa, Oryza sativa
  source 1..184: note = ssp. japonica
MGDNASASASVLAPPVGAGEGDAPSFSYLAALGNCPLVAAVLAGAIAQFIKVLTTWYKENRWDAKQLVGSGGMPSSHSATVVALAVAVGLQEGFGSSLFATAAIFASVVMYDAFGVRLHAGKQAEVLNQIVYELPSEHPLAETRPLRELLGHTPAQVFAGGVLGFAVATFTGMIAGLGNTGSLP

SEQ ID NO: 47 - protein, 341 aa, Picea abies
MGIAYTPRIHVHTRQQQVLLHNDFKVGSSNTGHFFRLKFEPLSKWPSSSQSHQNTSISLISVFKNKRRRRSSWALVHACAATTPFAPFVAVVTRVLQSILVGGEKGYYILRTTANKWVARLQEGYDMLRKAELANDVQEGYDMLRKAELANDGIGVLQQWGGGGGEEGIRIGMALLSTTTSMAKERISPVLGRLGANPTFMSGLLAWVIAQMLKVVTTFVVERRWDLRMLVGSGGMPSSHAALCVALTTSVALCHGVSDSLFPVCLGFSLIVMYDAAGVRRHAGMQAEVLNLIVEDLFKGHPVSEKKLKELLGHTPLQVFAGACLGVLVGYLCSHSCLVAL

SEQ ID NO: 48 - protein, 140 aa, Picea abies
MLSSPCNLLTTPRCSTHHVVQHQWLGHPIRLRHSKHYYSTKHSSACLGGPRAGLEQIAHNQVLVSATAACLIGQLSKPLASALLGKGFKWRLAVKSGGMPSTHSASIVASATAIGLERGFSDSLFGLSVVVAGIVMYDAQ

SEQ ID NO: 49 - protein, 61 aa, Picea abies
MLLLLGSGIGDSNPIRAVPMLSSPCNLLTTPRCSTHHVVQHQWLGHPIRLRHSKHYYSTFF

SEQ ID NO: 50 - protein, 93 aa, Picea abies
MSMYGKNVDSTQIGVKADNVALLTNVGRGCHLRYKEKRWDAKRLLGSGGMPSSHSASVTALAAAIGFHDGPGGSSFAISLVLACVVSLTIFSL

SEQ ID NO: 51 - protein, 171 aa, Picea abies
MQGVRRAVGKQAEVINMMIVSNTVPVCTDNNINSLVNDGEQILDSMQRELNSVEDMDPSVAISQIAVARAETIRSATCFPRETEVSSVRPHEIRISNEDGQFIDGAGLSSLSELTQTSPSVKTGKVDFQRLKQLAAWRHIPLKESVGHTKVEVLVGGLVGLIVTLGLQWIR

SEQ ID NO: 52 - protein, 327 aa, Physcomitrium patens
MACLALSRTAGPSTLYLPIGSPLHPRHLFRHFVPALDNANLHPRCALSRQGWSIICGTSWNHHARKETSVAGQQCCQSHSCSSFSEGRSNEDVSCSAGEDADKSPTHSPLHLTTFLAQALAIVSYWRARLHRCRAELETLCEESERETSEGALLMQGSMGMALLSISMIARDRISPVLITLRTNPTFMSGLVAWAIAQVLKVFTKYFVERRWDWKMLVGSGGMPSSHSALCVGLTTAVALCHGVGDSLFPVCLGFTLIVMYDAAGVRRHAGRQAEVLNMIVEDLFQGHPVSEKKLKELLGHTPLQVGAGAILGMICGYICSRSSMVY

SEQ ID NO: 53 - protein, 325 aa, Physcomitrium patens
MACLALSRITGPSMFSPPIASPPHSRSLVQHFDPTLNTATLHSRCFVPLQALSNTRQSSLKHRVLREKSVFGQQCCQSHGCSFGERDLNDDKQDFTGDDAILLRHLFFHPMTLLAQALSIISYWRARLHLCRLKFEALCEESQWEDLDGALLQQGSLGMALLSTSMIARDRISPVLITLRANPTFMSGLVAWAFAQVLKVFTKYFVERRWDWKMLVGSGGMPSSHSALCVGLTTAVALCHGVGDSLFPVCLGFTLIVMYDAAGVRRHAGRQAEVLNMIVEDLFQGHPVSEKKLKELLGHTPLQVGAGATLGMICGYICSRASMVY

SEQ ID NO: 54 - protein, 330 aa, Physcomitrium patens
MAARLLKHPAAPGACAHRNVGRRGEHDVAGQGIHRVGKLKLGGREWLGEVNVANARLQIKQRRRGDLRMQFRAGLEELASNQVLISAATASTLGQLAKPFAAALAGKGFNWKLVIKSGGMPSSHAASVTAAATALAFERGLSDGVFGLSVIIAGIVMYDAQGVRNAVGKQAKVINTMVVSYVPLPQSQEEESLSAATPSSAAIGAALARDPIEAELAFMDNTYTPSSSATQTQSSLATLEAGSPNASKSPVGAAADMNNGAYTRSSRFFELAGQLPSMRVGEVDIQELGNQDGWRLLPLKESIGHTKIEVLVGGIWGIVITCIFHSLYNS

SEQ ID NO: 55 - protein, 288 aa, Physcomitrium patens
MLKLGRDDWLGDGNFTHTRLLLKQKRRSCRIHVRAGIEELALDQVLVSAVTASTLAQLVKPFAAGLIGKGFNWKLIYKSGGMPSSHSAAVTAAATALAYERGLSDGVFGLSVIVACIVMYDAQGVRNAVGKQAKVINTMVVSYIPQPQPQPQPQVEQSLPVEIGAELTQDPAEMVRAVLDKNSSSKAMLNAPSLVILDGKSPSASKSYSETAADFYNGAYTRSRGFFELARQMPAMQVGEVDIQELGKQDGWRLLPLKESIGHTKIEVLGGGLWGVVITCILHSLHQS

SEQ ID NO: 56 - protein, 181 aa, Physcomitrium patens
MRMERLLAGDMAMASGDVAANLVNAQYDGFTNFPIVAAFLSFFVAQSLKVLTTWYKENRWDVKRLYGSGGMPSSHSATVTGLACAIGLREGLGGPLFAIAFVLACIVMYDASGVRLQAGRQAEVLNQIVFELPPEHPLSDSRPLKEFLGHTPPQVAAGAMLGCLIAYTLHLLSLIQAGGDK

SEQ ID NO: 57 - protein, 435 aa, Physcomitrium patens
MVSQIGIPLHPYENFTDGDLALLLQYTKEAYVERNGFSVADILENFPLWAALLAIGIAQFIKIPLNYFATKTWQWSLMLSTGGMPSSHSSAVTALSTAVGLREGFSSNMFAISAILGVIVMFDAAGVRRHAGMQAVVLNKLVDEFNHLLEGMKSLKVRPNQEKAKKLKELLGHQPIEVLIGGWLGIMIVVGLDDHSDWPLEWQHLPKVGPDLTIDMDRVAALKPDLVVASLSVPGMEANVEALQSRGIPHIVLNPSRINEIAADIRLVGEATDMHKQADQLATLFDERVETIRHRASQYSHRPKLYWEWWPNPIYTPGEENWLTDVSEIAGAVNVFRDYPVANVKATREMVAERDPDHICVVWCGIELKRIKPAMITERPEWQEMTAIRKHQVHLLEEGLYCRPSPRILDGLDKLAVEKGFCQLAITSSDDIDVH

SEQ ID NO: 58 - protein, 46 aa, Physcomitrium patens
VMYDALGVSLQAAPLHMLNQIVFEIPPEHPLLDSRPLKEVFGYTPP

SEQ ID NO: 59 - protein, 309 aa, Sequoiadendron giganteum
MGAFRSLRLDYWHSSLHNGFKIGGTDFLKVKHFKKKSVPSWSSRHHYHCTRIYVVKSNNDNGNRGNNWAFIHGVPPVAPMLAALALVLQNMRGRVMFSGEQGSYILQRTVKKWMARLQGCRNMVKEAELGNGRFLQQGGIGMALMSTTLMAKQKISPVLETLWANPTFMSGLLAWAIAQMLKVVTTFVVERRWDLKMFVSSGGMPSSHSALCCALTTSVALCHGVSDALFPVCLGFSLIVMYDAIGVRRHAGMQAEVLNLIVEDLFKGHPISDRKLKELLGHTPLQVIAGACLGVFVGYRCSRNCSVVF

SEQ ID NO: 60 - protein, 259 aa, Selaginella moellendorffii
MAIAAPLHCNWLGSRLSYRRMWISSSQRNLGAGRTVTAGLLEEIPQNHVLASAALAGLSAQLVKPLTAAVAGKGLNWKLMLRSGGTPSAHAASMVAAATALGLERGFSDSLFGFAMVVAGIVMYDAQGVRREVGKHAEILNTIAFAQYKVSKEPSPRSSRPELLVEAPVGATKSSNAFERGEVDSSNNGPFSRSTKFFKTAQNLPSMKEGEVSIQELGSEDGWQYIPLKESTGHTKSQVLAGAVFGAILSVISHAIGLS

SEQ ID NO: 61 - protein, 171 aa, Selaginella moellendorffii
MEGAVAAADQSLDPAAVPSAYSSFSNLPLVAAFVSFVAAQSLKIVTTWYKEKRWDLKRMAGSGGMPSSHSATVIGLTVAIGLRDGTGGSLFAIALVLASIVMYDASSVRFHAGRQAEVLNQIVFELPPEHPLADSRPLREPLGHTPPQVAAGAALGCIIAYILYLISLLGV

SEQ ID NO: 62 - protein, 157 aa, Selaginella moellendorffii
MASAHLSPVIVTLRANPTFMSGLVAWMVAQASKVLTTYVVYRRWDLRMLVGSGGMPSSHSALCLGLTTSVALSHGVGDALFPVCLGFSLIVMYDATGVRRHAGMQAEVLNMIIKDLFQGHPVSEKKLKELLGHTPLQVVAGALVGILVGWWCCLTPR

SEQ ID NO: 63 - protein, 266 aa, Solanum lycopersicum
MLSLQNWGYPSTVSLQNPSIYKNTTTKILVFPCLGKNGRCFSYSSLTSKPLPKLLCLHGFGVEDITDVVHNKVLVAAAVSAAVGQLMKPFTSSLFYGNEFDFKTAFQAGGFPSTHSSAVVATATALGLERGFSDSIFGLAVVYAGLVMYDAQGVRREVGIHAKAFNKALFRNQINSVPSTSELDVLTDSIQEKLSSEAENSDPQLSEESSSFQPRSKNATLLLKPDERRAPSSSFAPLKEQVGHTEVEVIAGAFLGFFVSLAVSLA

SEQ ID NO: 64 - protein, 286 aa, Solanum lycopersicum
MDSLLINSPYTALASSKILNSNITSFNLSSFCFFRFKRPNKLKTSSVTVSYLNPQNPNSEWQNHSSYLKPFSLLLPIFKKVKNFAENNRWGSVFKGCSGTENVPEELRGDLLQNGSFGMALLSITATAKVKISPIVATLAANPTFVSGFIAWFMAQSMKVFLNFCVERKWDFRIMFASGGMPSSHSALCTALTTSVAICHGVADSLFPVCLGFTLIVMYDAIGVRRHAGMQAEVLNLIVEDLFQGHPISQRKLKELLGHTPLQVFAGALLGIIVAWMCSQGYLIAI

SEQ ID NO: 65 - protein, 167 aa, Solanum lycopersicum
MMTATMTTTVSVGSSSFFTNYPLMSALIAFALAQSIKLFTSWYKERRWDLKQLVGSGGMPSSHSSTVTALAVAVGLQEGFGGALFACALVLACVVMYDATGVRLHAGRQAEVLNQILYELPSEHPLADSRPLRELLGHTPPQVVAGGLLGLTTATAIHFIRGSGHEA

SEQ ID NO: 66 - protein, 182 aa, Solanum lycopersicum
MMDSTVTLIQNLDPMNISTTTTTIASYGSSSFLSNCPLLSAIIAFALAQSIKFFTSWYREKHWDLKQLVGSGGMPSSHSSTVTALATAVGLQEGFGGSLFAISLVLACVVMYDATGVRLHAGRQAEVLNQIVCELPEEHPLADTLPLRELLGHTPPQVIAGGFLGLVTATIVWLITSSAYRA

SEQ ID NO: 67 - protein, 166 aa, Solanum lycopersicum
MNEVLTASDASSSARSYSSSIAPVNVPLFSALLACAIAQFLKLFTTWYKEKRWDSKRMLSSGGMPSSHSATVTSLIMAIYLQEGAGGSVFAIAVVLACVVMYDATGVRLHAGRQAELLNQIVCELPPEHPVANVRPLRDSLGHTPLQVLAGAVLGCVVPLLLRSSI

SEQ ID NO: 68 - DNA, 12883 bp, synthetic construct
  source 1..12883: mol_type = other DNA
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcggtacccggggatcctctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctggggacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 69 - protein, 133 aa, Arabidopsis thaliana
  source 1..133: note = Columbia-0 ecotype
NPTFVSAVVAWFFAQSSKMVINFFIERKWDFRLLYASGGMPSSHSALCMALTTSVALCHGVADSLFPVCLGFSLIVMYDAIGVRRHAGMQAEVLNLIIRDLFEGHPISQRKLKELLGHTPSQVLAGALVGIVI

SEQ ID NO: 70 - protein, 136 aa, Arabidopsis thaliana
  source 1..136: note = Columbia-0 ecotype
FTNYPLISAVTSFTIAQFIKLFTSWYRERRWDLKQLIGSGGMPSSHSATVTALAVAIGLQEGFGGSHFAIALILASVVMYDATGVRLHAGRQAEVLNQIVYELPAEHPLAESRPLRELLGHTPPQVVAGGMLGSAT

SEQ ID NO: 71 - protein, 136 aa, Arabidopsis thaliana
  source 1..136: note = Columbia-0 ecotype
FTNYPLISAVLAFTIAQFIKFFTSWYKERRWDLKRLVGSGGMPSSHSATVTALALAVGLQEGFGGSHFAIALVLTTIVMYDATGVRLHAGRQAEVLNQIVYELPAEHPLAETRPLRELLGHTPPQVIAGGMLGIST

SEQ ID NO: 72 - protein, 135 aa, Arabidopsis thaliana
  source 1..135: note = Columbia-0 ecotype
HNLPIFSAFLAFALAQFLKVFTNWYKEKRWDSKRMISSGGMPSSHSATVTALAVAIGFEEGAGAPAFAIAVVLACVVMYDASGVRLHAGRQAELLNQIVCEFPPEHPLSTVRPLRELLGHTPIQVAAGGILGCVV

SEQ ID NO: 73 - protein, 155 aa, Arabidopsis thaliana
  source 1..155: note = Columbia-0 ecotype
IHNKVLIAAGTSAVIGQLSKPFTSVVLYGKNLDFRSVFQAGGFPSTHSSSVVAAATAIAFERGFADSIFGLTVVYAGLIMYDAQGVRREVGKHAKVLNKLTANARRSEVMSLKGNESNKALTSEEISEEIAPPLKESIGHTEVEVIAGALFGFLV

SEQ ID NO: 74 - protein, 160 aa, Solanum lycopersicum
LLSITATAKVKISPIVATLAANPTFVSGFIAWFMAQSMKVFLNFCVERKWDFRIMFASGGMPSSHSALCTALTTSVAICHGVADSLFPVCLGFTLIVMYDAIGVRRHAGMQAEVLNLIVEDLFQGHPISQRKLKELLGHTPLQVFAGALLGIIVAWMCSQ

SEQ ID NO: 75 - protein, 155 aa, Solanum lycopersicum
TMTTTVSVGSSSFFTNYPLMSALIAFALAQSIKLFTSWYKERRWDLKQLVGSGGMPSSHSSTVTALAVAVGLQEGFGGALFACALVLACVVMYDATGVRLHAGRQAEVLNQILYELPSEHPLADSRPLRELLGHTPPQVVAGGLLGLTTATAIHF

SEQ ID NO: 76 - protein, 154 aa, Solanum lycopersicum
TTTTTIASYGSSSFLSNCPLLSAIIAFALAQSIKFFTSWYREKHWDLKQLVGSGGMPSSHSSTVTALATAVGLQEGFGGSLFAISLVLACVVMYDATGVRLHAGRQAEVLNQIVCELPEEHPLADTLPLRELLGHTPPQVIAGGFLGLVTATIV

SEQ ID NO: 77 - protein, 155 aa, Solanum lycopersicum
ASSSARSYSSSIAPVNVPLFSALLACAIAQFLKLFTTWYKEKRWDSKRMLSSGGMPSSHSATVTSLIMAIYLQEGAGGSVFAIAVVLACVVMYDATGVRLHAGRQAELLNQIVCELPPEHPVANVRPLRDSLGHTPLQVLAGAVLGCVVPLLLRS

SEQ ID NO: 78 - protein, 203 aa, Oryza sativa
  source 1..203: note = ssp. japonica
VEDITDVVHNKVLVAAAVSAAVGQLMKPFTSSLFYGNEFDFKTAFQAGGFPSTHSSAVVATATALGLERGFSDSIFGLAVVYAGLVMYDAQGVRREVGIHAKAFNKALFRNQINSVPSTSELDVLTDSIQEKLSSEAENSDPQLSEESSSFQPRSKNATLLLKPDERRAPSSSFAPLKEQVGHTEVEVIAGAFLGFFVSLAVS

SEQ ID NO: 79 - protein, 118 aa, Oryza sativa
  source 1..118: note = ssp. japonica
TLLMSTTAAAVTKARENPYILALAANPTFVSGLVAWAVAQAAKVVLTSFVERRWDLRMLFSSGGMPSSHTALCTALTASVALCHGVSDSLFPVCLGFTLIVMYDATGVRRHAGMQAEV

SEQ ID NO: 80 - protein, 144 aa, Oryza sativa
  source 1..144: note = ssp. japonica
AAAVVNYPLVAALVAFALAQSSKFFTTWFKEKRWDARQLIASGGMPSSHSATVTALAVAIGIQEGYRSATFATSVIIACVVMHDAFGVRLHAGKQAEVLNQIVYELPEEHPLSETKPLREILGHTVPQVVAGCIIGILIAVVMR

SEQ ID NO: 81 - protein, 147 aa, Oryza sativa
  source 1..147: note = ssp. japonica
SYFAVFHNYPLVAALLGFAVAQSIKFFVTRYKENRWDPKQLIGSGGMPSSHSATVTALAVAIGFQDGFGCALFATAAIFASVVMYDASGIRLHAGKQAEVLNQIVCELPSEHPLSETRPLRELLGHTPTQVVAGALLGSMLATAGQM

SEQ ID NO: 82 - protein, 134 aa, Oryza sativa
  source 1..134: note = ssp. japonica
NCPLVAAVLAGAIAQFIKVLTTWYKENRWDAKQLVGSGGMPSSHSATVVALAVAVGLQEGFGSSLFATAAIFASVVMYDAFGVRLHAGKQAEVLNQIVYELPSEHPLAETRPLRELLGHTPAQVFAGGVLGFAV

SEQ ID NO: 83 - protein, 71 aa, Oryza sativa
  source 1..71: note = ssp. japonica
IFFLQVMYDASGIRFHTGRQAALLNQIVSDFPPEHPIISSFRPLQEPLGHSPFQVFAGALVGCSIAYLMGK

SEQ ID NO: 84 - protein, 167 aa, Oryza sativa
  source 1..167: note = ssp. japonica
VAVATSLGLERGFADSIFGMSVVFAAIVMYDAQGVRREVGNHARVLNKLLTLREKITQNPDDNSLLSSTSELHSSKPETVAELVSVAEKLGSSQGSSANPFPIHSSGTKSSRLNALQSSETEVTEFTQLKEAYTEECDRLSESVGHTELQVAAGALLGFLVTLVVYA

(19) United States
(12) Patent Application Publication
(10) Pub. No.: US 2026/0098276 A1
(43) Pub. Date: Apr. 9, 2026

(54) COMPOSITION TO IMPROVE SALT TOLERANCE IN A PLANT OR ALGAE AND METHODS OF PRODUCTION THEREOF

(71) Applicant: ASSOCIAÇÃO BIOPOLIS, Vairão (PT)

(72) Inventors: Pedro Humberto ARAÚJO RAMOS FERNANDES CASTRO, Braga (PT); Leandro Miguel BASTOS PIRES, Fânzeres (PT); Herlander Anselmo QUEIRÓS PEREIRA AZEVEDO, Braga (PT); Tiago Jorge MOREIRA LOURENÇO, Guimarães (PT)

(73) Assignee: ASSOCIAÇÃO BIOPOLIS, Vairão (PT)

(21) Appl. No.: 19/139,669

(22) PCT Filed: Dec. 18, 2023
(86) PCT No.: PCT/IB2023/062890

§ 371 Date: Jun. 16, 2025

(30) Foreign Application Priority Data
Dec. 16, 2022 (PT) 118405

(51) Int. Cl. C12N 15/82; C07K 14/415; C12Q 1/6895

(52) U.S. Cl. CPC C12N 15/8273; C07K 14/415; C12N 15/8205; C12Q 1/6895; C12N 2830/50; C12Q 2600/13

(57) ABSTRACT

The present disclosure relates to the use of a composition for improving the tolerance of a plant or algae to salt, comprising a molecule, wherein the molecule inhibits an expression of a specific nucleotide sequence. Furthermore, the present disclosure relates to a method and kit to determining the tolerance of a plant or algae to salt.

### CROSS-REFERENCE TO RELATED APPLICATIONS

This application is a U.S. National Stage Application under 35 U.S.C. § 371 of International Patent Application No. PCT/IB2023/062890, filed Dec. 18, 2023, which claims priority to Portugal Patent Application No. 118405, filed Dec. 16, 2022, the contents of which are hereby incorporated by reference in their respective entireties.

### SEQUENCE LISTING

This instant application contains a Sequence Listing which has been submitted electronically in XML format and is hereby incorporated by reference in its entirety. Said XML copy, created on Dec. 3, 2025, is named 10224_013245-US0_SL-v2.xml and is 118,831 bytes in size.

### TECHNICAL FIELD

The present disclosure relates to the field of agricultural biotechnology, specifically addressing methods and compositions for conferring enhanced salt stress tolerance to plants. The disclosure involves the use of compositions and techniques designed to improve the ability of plants to thrive in saline environments, thereby contributing to increased crop yield and overall agricultural sustainability.

### BACKGROUND

As a consequence of climate change, plants will be more exposed to extreme and varying environmental conditions that will compromise plant fitness and survival. Moreover, climate change will potentiate desertification and degradation of the soils, ultimately creating unbalanced chemical compositions, including diverse salt ions (Marschner, 2012). This urges for sophisticated solutions, including genetic-based strategies to improve plant performance under deleterious saline conditions. The molecular mechanism for salt stress response in plants is very complex, involving multiple signalling pathways and adjusted ionic cell transport systems. For instance, the Salt Overly Sensitive (SOS) pathway and extracellular cation sensing by MONOCATION INDUCED [Ca²⁺]i INCREASES 1 (MOCA1) were ground-break discoveries in plant salt stress perception and response (Zhu, 2016; Jiang et al., 2019). The SOS pathway is so central that the high expression of SOS1 is a feature that confers salt tolerance to certain halophytes, such as Eutrema salsugineum (Oh et al., 2009). However, new evidences proved that the SOS pathway is highly regulated and a complex gene regulatory network exists to modulate its activity (Ali et al., 2023). Considering that the salt response is very intricate, involving both osmotic and ionic effects, many genetic players are yet to be discovered.

These facts are disclosed in order to illustrate the technical problem addressed by the present disclosure.

### General Description

Based on phylogenetic studies and salt-stress response transcriptomes, an uncharacterized gene family in plants named Salt-Expressed and Associated (SEA), with five members in Arabidopsis thaliana (hereafter Arabidopsis) is disclosed. Phylogenetic and functional investigations have uncovered that this family is ancestral, is involved in plant development (i.e. plant growth, flowering time), and in the response to different salts. The conservation of this family allows the design of breeding strategies to create plants more tolerant to saline conditions for diverse purposes including producing salt-tolerant crop varieties, promoting salt phytoremediation, and organic-salt biofortification production.

The present disclosure relates to the use of a composition for improving the tolerance of a plant or algae to salt, relative to a wild-type, wherein the composition comprises a molecule that reduces the expression of a nucleotide sequence encoding an amino acid sequence comprising at least a sequence 85% identical to the sequences of the following list SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84.

In an embodiment for better results, the amino acid sequence comprises at least a sequence 90% identical to the sequences of the following list SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84; preferably 95% identical, 96% identical, 97% identical, 98% identical, 99% identical or identical.

In an embodiment for better results, the nucleotide sequence encodes an amino acid sequence comprising at least a sequence 90% identical to the sequences of the following list SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, or homologue thereof.

In an embodiment for better results, the nucleotide sequence encodes an amino acid sequence comprising at least a sequence 95% identical to the sequences of the following list: SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, or a homologue thereof; preferably 96% identical, 97% identical, 98% identical, 99% identical or identical.

In an embodiment for better results, the nucleotide sequence is a gene comprising at least a sequence 90% identical to the sequences of the following list SEQ ID No 16, SEQ ID No 17, SEQ ID No 18, SEQ ID No 19, SEQ ID No 20, or a homologue thereof.

In an embodiment for better results, the gene comprises at least a sequence 95% identical to the sequences of the following list SEQ ID No 16, SEQ ID No 17, SEQ ID No 18, SEQ ID No 19, SEQ ID No 20, or homologue thereof; preferably 96% identical, 97% identical, 98% identical, 99% identical or identical.

In an embodiment for better results, the amino acid sequence comprises at least a sequence 85% identical to the sequences of the following list SEQ ID No 69, SEQ ID No 70, SEQ ID No 71, SEQ ID No 72, SEQ ID No 73; preferably 86% identical, 87% identical, 88% identical, 89% identical, 90% identical, 91% identical, 92% identical, 93% identical, 94% identical, 95% identical, 96% identical, 97% identical, 98% identical, 99% identical or identical.

In an embodiment for better results the amino acid sequence comprises at least a sequence 90% identical to the sequences of the following list SEQ ID No 26, SEQ ID No 27, SEQ ID No 28, SEQ ID No 29, SEQ ID No 30; preferably 91% identical, 92% identical, 93% identical, 94% identical, 95% identical, 96% identical, 97% identical, 98% identical, 99% identical or identical.

In an embodiment for better results the molecule is a protein molecule, a nucleic acid molecule or combinations thereof.

In an embodiment for better results the nucleic acid molecule is selected from a T-DNA, a siRNA, a shRNA, a miRNA, a ribozyme, a peptide nucleic acid, sgRNA, or antisense oligonucleotide.

In an embodiment for better results the protein molecule is selected from a zinc-finger nuclease, transcription activator-like effector nuclease, or CRISPR-associated protein 9.

In an embodiment for better results the nucleic acid molecule inhibits the expression of the nucleotide sequence by insertion in said nucleotide sequence.

In an embodiment for better results the composition comprises Agrobacterium tumefaciens cells comprising the nucleic acid molecule.

In an embodiment for better results the composition comprises a recombinant plant expression vector comprising the nucleic acid molecule or a nucleic acid that generates an RNA molecule encoding said protein molecule in plant cells; and optionally a poly A signal sequence inducing polyadenylation at the 3′-end of the RNA molecule.

In an embodiment for better results the use is as a salt tolerance improver of a plant.

In an embodiment for better results the plant or algae is selected from Arabidopsis thaliana, Amborella trichopoda, Chlamydomonas reinhardtii, Medicago truncatula, Oryza sativa, Picea abies, Physcomitrium patens, Sequoiadendron giganteum, Selaginella moellendorffii, or Solanum lycopersicum.

In an embodiment for better results the salt is sodium chloride or potassium chloride.

An aspect of the present disclosure relates to the use of at least a sequence according to the disclosure for the manufacture of a composition for increasing plant/algae salt tolerance.

The present disclosure also relates to a method for producing a genetically modified plant or algae with increased salt tolerance relative to a wild-type, the method comprising the following steps: introducing at least one mutation or exogenous nucleic acid into the genome of one or more plant or algae cells which results in reduced activity associated with a protein, wherein the protein comprising at least a sequence 90% identical to the sequences of the following list SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, or a homologue thereof in said one or more plant or algae cells; regenerating one or more plants or algae from said one or more plant or algae cells; and selecting one or more plants or algae that have increased salt tolerance relative to a wild-type. Preferably, the method is for producing a plant.

In an embodiment for better results the method comprises introducing at least one mutation into a gene comprising at least a sequence 90% identical to the sequences of the following list SEQ ID No 16, SEQ ID No 17, SEQ ID No 18, SEQ ID No 19, SEQ ID No 20, or homologue thereof, or inhibiting or suppressing the expression of said gene or homologue thereof; preferably 91% identical, 92% identical, 93% identical, 94% identical, 95% identical, 96% identical, 97% identical, 98% identical, 99% identical or identical.

In an embodiment for better results the exogenous nucleic acid is a T-DNA.

In an embodiment for better results the T-DNA introduction into the genome of one or more plant or algae cells is facilitated by Agrobacterium tumefaciens-mediated transformation.

In an embodiment for better results the exogenous nucleic acid comprises a nucleic acid complementary to at least a portion of the encoding sequence, or homologue thereof, of a protein comprising at least a sequence 90% identical to the sequences of the following list SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67.

In an embodiment for better results the disclosed method comprises a step of transforming a plant, plant tissue culture, or plant cell or algae with a vector comprising the exogenous nucleic acid.

In an embodiment for better results the vector is a binary vector, a virus derived vector, a plasmid, a liposome, a dendrimer, or nanoparticle vector; preferably a plasmid vector.

In an embodiment for better results the vector comprises a sequence at least 90% identical to SEQ ID. No 68, preferably 95% identical, more preferably 96% identical, 97% identical, 98% identical, 99% identical or identical.

In an embodiment for better results the resulting plant has increased tolerance to salt relative to a wild-type plant.

In an embodiment for better results the plant is selected from Arabidopsis thaliana, Amborella trichopoda, Chlamydomonas reinhardtii, Medicago truncatula, Oryza sativa, Picea abies, Physcomitrium patens, Sequoiadendron giganteum, Selaginella moellendorffii, or Solanum lycopersicum.

The present disclosure also relates to a method for screening a plant with increased salt tolerance relative to a wild-type plant, the method comprising analysing DNA of the plant for the presence of at least one allele of a nucleotide sequence encoding a protein comprising at least a sequence 90% identical to the sequences of the following list SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67 or a homologue thereof, using at least one nucleic acid molecule suitable as a probe or primer which is capable of hybridising to a gene comprising at least a sequence 90% identical to the sequences of the following list SEQ ID No 16, SEQ ID No 17, SEQ ID No 18, SEQ ID No 19, SEQ ID No 20, or a homologue thereof.

In an embodiment for better results the method comprises the use of at least one oligonucleotide primer pair suitable for amplification of a region of a gene comprising at least a sequence 90% identical to the sequences of the following list SEQ ID No 16, SEQ ID No 17, SEQ ID No 18, SEQ ID No 19, SEQ ID No 20, or homologue thereof, said primer pair comprising a forward primer and a reverse primer to detect the presence or absence of a mutation in said region.

In an embodiment for better results the method comprises the following steps: obtaining a biological sample from a plant; contacting the sample with the at least one oligonucleotide primer pair; performing a nucleic acid amplification reaction; measuring the level of expression of a gene comprising at least a sequence 90% identical to the sequences of the following list: SEQ ID No 16, SEQ ID No 17, SEQ ID No 18, SEQ ID No 19, SEQ ID No 20, or homologue thereof; comparing the level of expression of the gene in the biological sample with the level of expression of the gene in the wild-type sample; wherein a lower level of expression of the gene corresponds to a higher tolerance to salt.

In an aspect the present disclosure relates to a kit for determining the tolerance of a plant or algae to salt comprising nucleotide components capable of detecting one or more nucleotide sequences comprising at least a sequence 90% identical to the sequences of the following list: SEQ ID No 16, SEQ ID No 17, SEQ ID No 18, SEQ ID No 19, SEQ ID No 20, and complements thereof.

In an embodiment for better results the kit comprises an oligonucleotide primer pair suitable for amplification of a region of a gene comprising at least a sequence 90% identical to the sequences of the following list SEQ ID No 16, SEQ ID No 17, SEQ ID No 18, SEQ ID No 19, SEQ ID No 20, or homologue thereof, said primer pair comprising a forward primer and a reverse primer to detect the presence or absence of a mutation in said region.

The present disclosure also relates to a plant with increased salt tolerance relative to a wild-type plant, obtained by the disclosed method, with the proviso that said plant is not an Arabidopsis thaliana SALK_030394, or SALK_039758 mutant.

In an embodiment for better results said plant is selected from Arabidopsis thaliana, Amborella trichopoda, Chlamydomonas reinhardtii, Medicago truncatula, Oryza sativa, Picea abies, Physcomitrium patens, Sequoiadendron giganteum, Selaginella moellendorffii, or Solanum lycopersicum.

In an embodiment for better results the resulting plant has an earlier flowering time relative to a wild-type plant.

### BRIEF DESCRIPTION OF THE DRAWINGS

The following figures provide preferred embodiments for illustrating the disclosure and should not be seen as limiting the scope of invention.

FIG. 1: Embodiment of phylogenetic reconstruction and topological analysis of the plant Salt-Expressed and Associated (SEA) family. (A) The phylogenetic analysis was performed using the protein sequence of different Green Plant species (i.e. Arabidopsis thaliana, AT; Medicago truncatula, Medtr; Solanum lycopersicum, Solyc; Oryza sativa, Os; Amborella trichopoda, ATR; Sequoiadendron giganteum, SEGI; Picea abies, PAB; Selaginella moellendorffii, SMO; Physcomitrium patens, Pp) and the model algae species (Chlamydomonas reinhardtii, Cre), and calculated using maximum-likelihood with bootstrap values from 1000 replicates. (B) Schematic representation of Arabidopsis SEA proteins topology, highlighting the DUF212 domain, also known as acid phosphatase/vanadium-dependent haloperoxidase-related. Scale represents 100 amino acids. (C) Protein sequence alignment of the DUF212 domain in Arabidopsis SEAs. Consistency scale indicates the level of conservation of each amino acid residue.

FIG. 2: Embodiment of plant SEA gene expression levels in plants and in response to salt stress. (A) The absolute expression values of each AtSEA were obtained from RNA-Seq data available at BAR database and average calculated for all developmental stages and tissues. (B) The AtSEAs relative expression values in 10-day-old Arabidopsis wild-type (Columbia-0 ecotype) seedlings growing under salt stress (100 mM NaCl) were obtained by quantitative RT-PCR (qPCR) analysis. Arabidopsis ACT2 was used as a reference gene. Error bars indicate standard deviation of the means. Asterisks represent statistically differences between salt treated plants and control plants (unpaired t test; ns-non-significant, *P<0.05, ***P<0.001).

FIG. 3: Embodiment of characterization of AtSEA1 mutants. (A) Schematic representation of AtSEA1 (AT3G1770) and location of the T-DNA insertion sites (inverted triangles). The promoter, UTRs, and exons are represented by white, black, and striped boxes, respectively. Scale represents 100 base pairs. (B) A qPCR analysis was performed to access the AtSEA1 relative expression levels in the wild-type (WT, Col-0 background) and sea1 mutant lines backgrounds. (C) The flowering day was scored for WT and mutants (n=12). (D) The stem length was measured in 1-month-old plants (n=12). Error bars represent standard error of the means. Asterisks represent statistically differences between mutants and WT (unpaired t test; *P<0.05, ***P<0.001).

FIG. 4: Embodiment of characterization of AtSEA1 knock-out lines in response to salt stress. (A) Seedling morphology two weeks after germination, cultured in different conditions (Control, 100 mM NaCl and 100 mM KCl). (B) Shoot fresh weight of 2-week-old seedlings exposed to different salt supplementation (i.e. 100 mM of NaCl or KCl) conditions (n≥3 plates). Error bars represent standard error of the means. Asterisks represent statistical differences between mutants and WT in each condition (unpaired t test; ns-non-significant, *P<0.05, **P<0.01, ***P<0.001).

### DETAILED DESCRIPTION

The present disclosure relates to the use of a composition for improving the tolerance of a plant to salt, comprising a molecule, wherein the molecule inhibits an expression of a specific nucleotide sequence. Furthermore, the present disclosure relates to a method and kit to determining the tolerance of a plant to salt.

In an embodiment, it is disclosed the use of a composition that allows to improve a tolerance of a plant to an abiotic stress, in particular high salt concentrations, as compared to a wild-type control plant. It is shown that a nucleotide sequence comprising at least a sequence 90% identical to the sequences of SEQ ID No 16, SEQ ID No 17, SEQ ID No 18, SEQ ID No 19, SEQ ID No 20, or combinations thereof was involved in the above-mentioned characteristic of the plant. In addition, where the expression of said nucleotide sequences was inhibited, genetically modified plants having improved tolerance to a salt stress condition were obtained.

It would be obvious to the skilled person that the nucleotide sequences used in this disclosure are not limited to those listed in the appended Sequence Listing.

For nucleotides, the variations may be purely genetic, i.e., ones that do not result in changes in the protein product. This includes nucleic acids that contain functionally equivalent codons, or codons that encode the same amino acid, such as six codons for arginine or serine, or codons that encode biologically equivalent amino acids.

In an embodiment, genetically modified plants can be obtained by different methods known to those skilled in the art. In an embodiment, the plant may be genetically modified by Agrobacterium-mediated transformation, particle bombardment transformation, electroporation, protoplast transformation, CRISPR-Cas9, RNA interference, virus-mediated transfer, Transcription Activator-Like Effector Nucleases (TALENs), or zinc finger nucleases (ZNF).

To introduce a foreign nucleotide sequence into plant cells or plants may be performed by the methods known to those skilled in the art. In an embodiment, the plant may be transformed using a foreign nucleotide sequence inserted into a carrier (e.g., vectors such as plasmid or virus) or Agrobacterium tumefaciens as a mediator (Chilton et al., Cell, 11:263:271 (1977)) and by directly inserting the foreign nucleotide sequence into plant cells (Lorz et al., Mol. Genet., 199:178-182 (1985)). In an embodiment, electroporation, microparticle bombardment, or polyethylene glycol-mediated uptake may be used.

In a preferred embodiment, Agrobacterium-mediated transformation is used.

In an embodiment, the introduction of a recombinant vector into Agrobacterium can be carried out by a large number of methods known to one skilled in the art. For example, transfection, particle bombardment, lithium acetate method, electroporation, vacuum infiltration, or heat shock method may be used.

In an embodiment, the vector is selected from a group of non-viral vectors, selected from the group of plasmid, liposome, dendrimer, or nanoparticle vector. In an embodiment for better results, the vector is a plasmid.

In an embodiment, the vector is a pROK2 vector. In an embodiment for better results, the vector comprises a sequence at least 90% identical to SEQ ID No. 68, preferably a sequence 91% identical, 92% identical, 93% identical, 94% identical, 95% identical, 96% identical, 97% identical, 98% identical, 99% identical or identical.

In an embodiment, the invention describes an isolated or artificial sequence or a variant thereof.

The variants include, for instance, naturally-occurring variants due to allelic variations between individuals (e.g., polymorphisms), alternative splicing forms, among others. The term variant also includes gene sequences from other sources or organisms. Variants are preferably substantially homologous to the nucleotide sequence of SEQ ID No 16, SEQ ID No 17, SEQ ID No 18, SEQ ID No 19, SEQ ID No 20, exhibit a nucleotide sequence identity of typically at least 90%, preferably at least 95%, preferably at least 96%, preferably at least 97%, more preferably at least 98%, more preferably at least 99%; preferably identical to the referred sequence.

Methods for the alignment of sequences for comparison are well known in the art, such methods include GAP, BESTFIT, BLAST, Clustal Omega. The BLAST algorithm (Altschul et al. (1990) J Mol Biol 215:403-10) calculates percent sequence identity and performs a statistical analysis of the similarity between the two sequences. The software for performing BLAST analysis is publicly available through the National Centre for Biotechnology Information (NCBI). Clustal Omega is a multiple sequence alignment program that uses seeded guide trees and HMM profile-profile techniques to generate alignments between three or more sequences (Sievers F, Wilm A, Dineen D, et al. (2011) Molecular Systems Biology 7:539), and it is publicly available through EMBL-EBI services (https://www.ebi.ac.uk/Tools/msa/clustalo/). Minor manual editing may be performed to optimise alignment between conserved motifs, as would be apparent to a person skilled in the art. The amino acid sequence identity values, which are indicated in the present subject matter as a percentage, were determined over the entire amino acid sequence, using Clustal Omega with the default parameters.

In an embodiment, SEQ ID No 16, SEQ ID No 17, SEQ ID No 18, SEQ ID No 19, and SEQ ID No 20 can also be defined by their respective AGI code. The AGI code is a unique identifier for each gene in the Arabidopsis genome, and it is used to link genes to their corresponding information in the TAIR database.

The following table describes the nucleotide sequences and respective AGI codes.

| SEQ ID No | AGI code |
|---|---|
| 16 | AT3G61770 |
| 17 | AT1G24350 |
| 18 | AT1G67600 |
| 19 | AT3G21610 |
| 20 | AT3G12685 |

In an embodiment, the Arabidopsis lines used were in the Columbia-0 (Col-0) ecotype genetic background. The T-DNA insertion lines for AtSEA1 (i.e. sea1-1, SALKseq_030394.1 (NASC ID: N530394); and sea1-2, SALKseq_039758.1 (NASC ID: N539758)) were requested from the Nottingham Arabidopsis Stock Centre (NASC). The genotypes were confirmed by diagnostic PCR using genotyping primers (AtSEA1 LP1 (SEQ ID No 1), AtSEA1 RP1 (SEQ ID No 2), AtSEA1 LP2 (SEQ ID No 3), AtSEA1 RP2 (SEQ ID No 4), AtSEA1 RP3 (SEQ ID No 5)), which confirmed that in these lines the SEQ ID No 16 was interrupted by insertional mutagenesis. All seeds were synchronized prior to any assay, and then stored in darkness at room temperature in microtubes. Arabidopsis seeds were stratified by immersion in ultrapure water and incubated at 4° C. for 3 days in the dark. Seed surface sterilization was achieved by submersion in 70% (v/v) ethanol for 5 min, removal of ethanol and further submersion in bleach (20% (v/v) commercial bleach with 3.5% (w/v) effective chloride) for 10 min. To clean the seeds of residual bleach, they were rinsed in sterile ultrapure water three times. After water rinsing, seeds were suspended in 0.2% (w/v) agarose solution. Sterilized seeds were dispersed onto plates containing 0.5 Murashige and Skoog (MS) medium. For germination studies 0.8% (w/v) agar concentration was used for horizontal growth, while for vertical growth and subsequent transplanting a 1.2% (w/v) agar concentration was used. Media had their pH adjusted to 5.7 using KOH 1 M and were autoclaved for 20 min at 121° C. and 1 atm. Plates were sealed with parafilm to prevent desiccation and placed horizontally in a growth room under the following conditions: photoperiod of 16 h light/8 h darkness; under cool white light (80 μE m⁻² s⁻¹ light intensity) and 22-23° C. The MS medium was made using 2.151 g L⁻¹ of MS (0.5×), 0.5 g L⁻¹ of MES, 15 g L⁻¹ of sucrose and 1.2 or 0.8% (w/v) of agar depending if the plates were vertical or horizontal, respectively. To induce salt stress, NaCl or KCl were added to MS media at a concentration of 100 mM. For regular growth, 7-day-old seedlings were transferred from 1.2% (w/v) agar plates to pots with a soil to vermiculite ratio of 4:1, with the soil previously sifted. The same growth room conditions were applied with regular watering every 2 to 3 days. Developmental was characterized according to Boyes et al. (2001). Table 1 indicates the day and morphological measurement.

TABLE 1
Morphological measurements and respective day after
stratification. Days of measurement were selected
according to Boyes's key developmental stages for
Arabidopsis thaliana (Boyes et al. (2001)).

| Day | Measurements |
|---|---|
| Day 17 | Rosette radius and number of leaves |
| Bay 24 | Rosette radius and number of leaves |
| Day 26 | Number of flower buds; rosette radius and number of leaves |
| Day 32 | Stem length; number of flower buds; rosette radius and numbers |
| | of leaves |
| Day 44 | Length of stem |

For the scope and interpretation of the present disclosure it is defined that “room temperature” should be regarded as a temperature between 15-30° C., preferably between 18-25° C., more preferably between 20-22° C.

In an embodiment, the RNA was extracted from plant tissue using RNeasy Plant Mini kit (QIAGEN) following the manufacturer's instructions. RNA quantity and integrity were assessed using the Nanodrop ND-1000 spectrophotometer and agarose-gel electrophoresis. RNA samples were treated with RNase-free DNAse I (ThermoScientific) to remove contaminant DNA. The cDNA synthesis was performed using NZY First-Strand cDNA Synthesis kit (NZYTech). The qPCR reactions were carried out with NZYSupreme qPCR Green Master Mix (NZYTech) as per the manufacturers' instructions. Reactions were performed in a MyiQ CFX96tm real-time system C1000 Touch thermal cycler (BioRad). Primers used for qPCR were AtSEA1 qPCR F1 (SEQ ID No 6), AtSEA1 qPCR R1 (SEQ ID No 7), AtSEA2a qPCR F1 (SEQ ID No 8), AtSEA2a qPCR R1 (SEQ ID No 9), AtSEA2b qPCR F1 (SEQ ID No 10), AtSEA2b qPCR F1 (SEQ ID No 11), AtSEA2c qPCR F1 (SEQ ID No 12), AtSEA2c qPCR R1 (SEQ ID No 13), AtSEA3 qPCR F1 (SEQ ID No 14), and AtSEA3 qPCR R1 (SEQ ID No 15). Arabidopsis ACT2 was used as a reference gene and relative expression values calculates as previously described (Castro et al., 2018).

In an embodiment, the National Center for Biotechnology Information (NCBI; https://www.ncbi.nlm.nih.gov/) database was used for sequence retrieval and primer design [Primer-BLAST tool, (Ye et al., 2012)] as described previously (Castro et al., 2018). Arabidopsis SEA genomic sequences (SEQ ID No 16 to 20) and coding sequences (SEQ ID No 21 to 25) were obtained from The Arabidopsis Information Resource (TAIR) database (https://www.arabidopsis.org/). For the construction of the phylogenetic trees, the web-based platform Dicots PLAZA version 5.0 (Van Bel et al., 2022) was used to obtain protein sequences of target gene homologs from multiple species (SEQ ID No 26 to 67). Subsequently, the phylogenetic tree was calculated using Maximum Likelihood inference with 1000 bootstraps, in the MEGA version 11 software (Tamura et al., 2021). The expression levels for each gene were retrieved from the Arabidopsis eFP browser (BAR database, http://bar.utoronto.ca/efp/cgi-bin/efpWeb.cgi). Graphics and statistical analysis of the data were performed using the GraphPad Prism version 6 software.

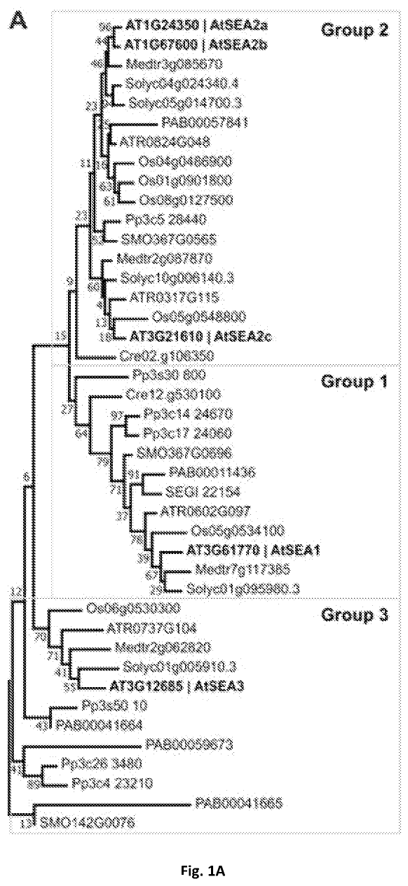

Salt-Expressed and Associated (SEA) incorporates a gene family in plants without any documented functional association. In an embodiment to understand the conservation of this gene family across plants, it was performed a phylogenetic study using the SEA amino acidic sequences (SEQ ID No 26 to 67) of representative major taxa species in Green Plants. The model plant Arabidopsis, which harbours the most extensive functional knowledge in plant species, was used as an anchor species for phylogenetic insight. All analysed species presented at least one SEA gene/protein (FIG. 1A). Moreover, it was observed that this family can be divided into three groups/clades (FIG. 1A). The Group 1 contains Arabidopsis thaliana SEA1 (AtSEA1, AT3G61770), present from green algae up to higher plants. The Group 2 is also present from green algae up to higher plants, and includes a recently duplicated gene pair in Arabidopsis, AtSEA2a (AT1G24350) and AtSEA2b (AT1G67600), plus the AtSEA2c (AT3G21610) Arabidopsis gene. In contrast, Group 3 does not have representatives in the algae Chlamydomonas reinhardtii, but it has representatives across plant taxa. Group 3 has a unique member in Arabidopsis, named AtSEA3 (AT3G12685).

In an embodiment, all Arabidopsis proteins contain a conserved domain registered in the protein databases (i.e. Expasy-PROSITE and InterPro) as Domain of Unknown Function 212 (DUF212, SEQ ID No 69, SEQ ID No 70, SEQ ID No 71, SEQ ID No 72, SEQ ID No 73) that resembles Acid Phosphatase and Vanadium-dependent Haloperoxidase structures (FIG. 1B-C).

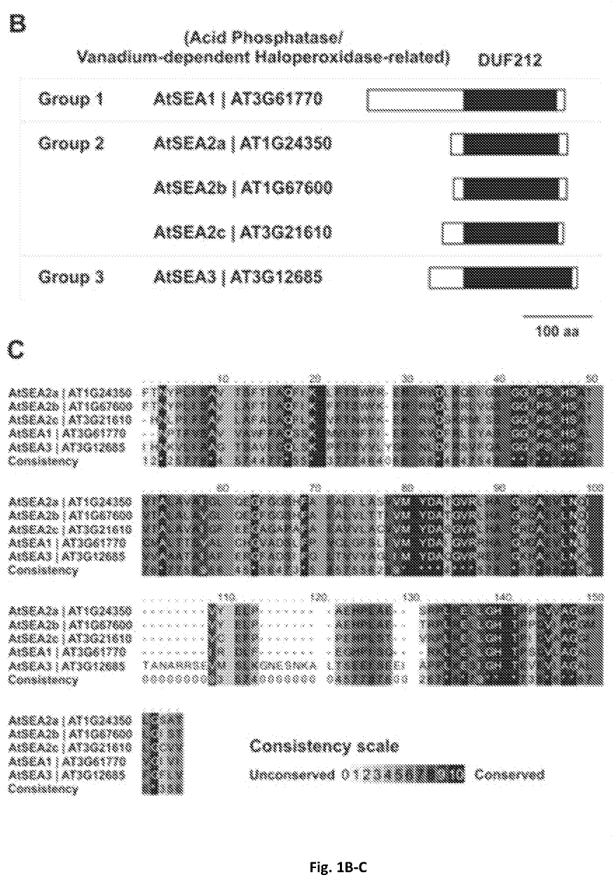

The five Arabidopsis SEAs are broadly expressed in plant tissues (FIG. 2A). In addition, with exception of AtSEA3 (SEQ ID No 25) and AtSEA2c (SEQ ID No 24), Arabidopsis transcripts are upregulated by NaCl treatment (FIG. 2B). It is noteworthy to mention that it was observed that AtSEA2c (SEQ ID No 24) presented a trend to increase, but without statistical differences. Therefore, it is possible to infer that SEA gene family members respond to high salt dosages and are involved in the molecular mechanisms of the saline stress response.

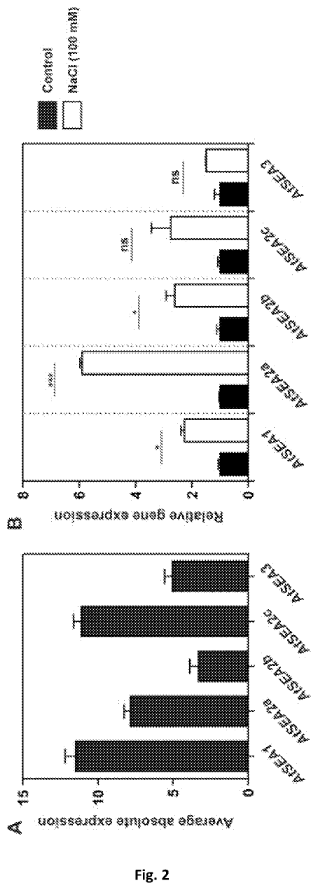

In an embodiment to further study the function of SEA genes, two independent AtSEA1 T-DNA insertion lines (FIG. 3A) were used (sea1-1 and sea1-2), that abolished AtSEA1 expression (FIG. 3B).

In an embodiment, sequence-indexed T-DNA insertion line was generated by vacuum infiltration of Columbia (Col) plants with Agrobacterium tumefaciens vector pROK2; kanamycin was employed for selection of plants carrying a T-DNA; each T1 transformant has been maintained individually; the DNA sequence of each T-DNA flanking region was generated from seedlings grown from the same sample of seeds as that provided for distribution (T3).

Surprisingly, it was observed that both sea1-1 and sea1-2 knockout lines displayed early flowering phenotypes in comparison to the wild-type (FIG. 3C-D). Salt tolerance mechanisms may induce stress responses in the plant. Flowering can be perceived as a plant's survival strategy, accelerating its reproductive phase as a response to stress such as salt stress.

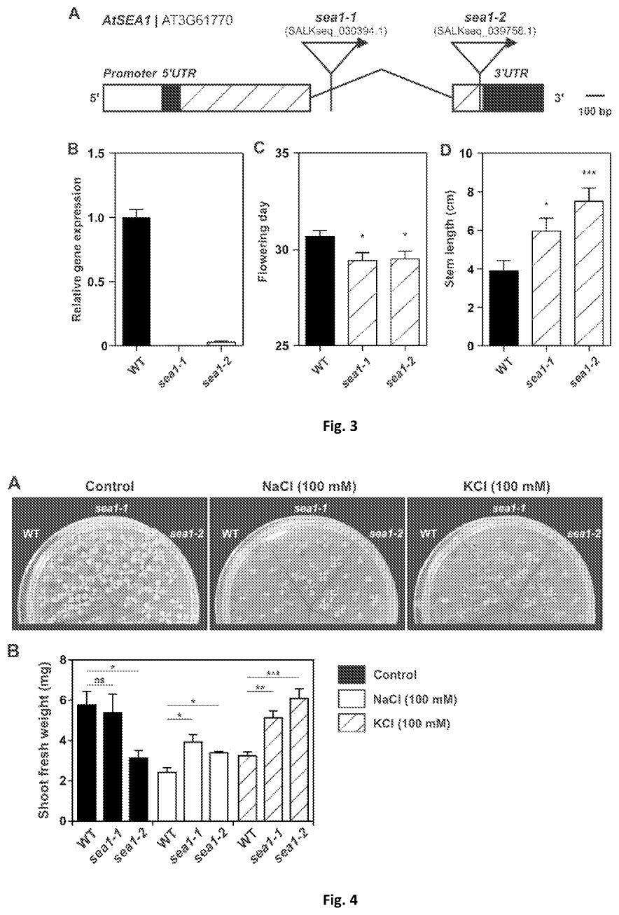

In an embodiment to further study the implications of SEA in salt response, Arabidopsis WT seeds were germinated with synchronized seeds of AtSEA1 homozygous knockout T-DNA lines. Under control conditions all genotypes germinated and produced comparable shoots. As expected, in high doses of salt supplementation (i.e. 100 mM of NaCl or 100 mM KCl), the wild-type seedlings displayed impaired growth (FIG. 4A-B). Surprisingly, both AtSEA1 T-DNA lines, i.e. lines where the expression of SEQ ID No 16 was inhibited by insertional mutagenesis, showed improved performance in high salt stress conditions, indicating that AtSEA1 is acting as a negative regulator of salt tolerance.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above-described embodiments are combinable.

As used herein the term “gene”, refers to a defined region that is located within a genome and that may comprise regulatory, nucleic acid sequences responsible for the control of expression, i.e., transcription and translation of the coding portion. A gene may also comprise other 5′ and 3′ untranslated sequences and termination sequences. Further elements that may be present are, for example, introns.

As used herein, the term “homologue” in the context of proteins means proteins having substantially the same functions and similar properties in different species, and which, within at least regions, share at least 50% amino acid identity. Such homologous proteins may share, over their entire amino acid sequences, at least about 30% amino acid identity, at least about 40% amino acid identity, at least about 50% amino acid identity, at least about 60% amino acid identity, at least about 70% amino acid identity, at least about 80% amino acid identity, at least about 90% amino acid identity or at least about 95% identity. Similarly, homologues of nucleic acid molecules are nucleic acid molecules that encode proteins having substantially the same functions and similar properties in different species, wherein the encoded proteins share, within at least regions, at least 50% amino acid identity (such nucleic acid homologues may share significantly less than 50% identity due to degeneracy in the genetic code, and differences in preferred codon usage amongst different plant genuses and species), and may share at least about 30% amino acid identity, at least about 40% amino acid identity, at least about 50% amino acid identity, at least about 60% amino acid identity, at least about 70% amino acid identity, at least about 80% amino acid identity, at least about 90% amino acid identity or at least about 95% identity over the whole encoded amino acid sequences.

As used herein, the term “mutation” means any change in a polypeptide or nucleic acid molecule relative to a wild-type polypeptide or nucleic acid molecule from which the “mutant” is derived and may, for example, comprise single or multiple amino acid or nucleotide changes, or both nucleotide and amino acid changes, including point mutations, null mutations, frame-shift mutations, and may comprise deletions, or insertions, or substitutions of one or more nucleic acids or amino acids, which may comprise naturally or non-naturally occurring nucleotides or amino acids or analogues thereof.

A “nucleic acid”, as referred to herein, refers to deoxyribonucleotides or ribonucleotides and polymers thereof in either single-, double-stranded or triplexed form. The term may encompass nucleic acids containing known analogues of natural nucleotides having similar binding properties as the reference nucleic acid. A particular nucleic acid sequence may also implicitly encompass conservatively modified variants thereof (e.g. degenerate codon substitutions) and complementary sequences. The terms “nucleic acid”, “nucleic acid sequence” or “polynucleotide” may also be used interchangeably with gene, cDNA, and mRNA encoded by a gene.

The terms “polypeptide”, “peptide” and “protein” may be used interchangeably herein to refer to a polymer of amino acid residues. Included within the scope of these terms are polymers in which one or more amino acid residues may comprise artificial chemical analogue(s) of corresponding naturally occurring amino acid(s), as well as, or instead of naturally occurring amino acid polymers. The terms “polypeptide”, “peptide” and “protein” may also include polymers including modifications such as, but not limited to, glycosylation, lipid attachment, sulfation, gamma-carboxylation of glutamic acid residues, hydroxylation and ADP-ribosylation.

The term “plant(s)” as used herein, is understood by a meaning including a plant cell, a plant tissue and a plant seed as well as a mature plant.

In the context of the present disclosure, “G, “C”, “A,” “T,” and “U”, each generally stand for a nucleotide that contains guanine, cytosine, adenine, thymidine, and uracil as a base, respectively.

The following dependent claims further set out particular embodiments of the disclosure.

### REFERENCES

Ali A, Petrov V, Yun D J, Gechev T (2023) Revisiting plant salt tolerance: novel components of the SOS pathway. Trends Plant Sci 28:1060-1069
Boyes D C, Zayed A M, Ascenzi R, Mccaskill A J, Hoffman N E, Davis K R, Gorlach J (2001) Growth stage-based phenotypic analysis of Arabidopsis: a model for high throughput functional genomics in plants. Plant Cell 13:1499-1510
Castro P H, Santos M A, Freitas S, Cana-Quijada P, Lourenco T, Rodrigues MAA, Fonseca F, Ruiz-Albert J, Azevedo J E, Tavares R M, Castillo A G, Bejarano E R, Azevedo H (2018) Arabidopsis thaliana SPF1 and SPF2 are nuclear-located ULP2-like SUMO proteases that act downstream of SIZ1 in plant development. J Exp Bot 69:4633-4649
Jiang Z, Zhou X, Tao M, Yuan F, Liu L, Wu F, Wu X, Xiang Y, Niu Y, Liu F, Li C, Ye R, Byeon B, Xue Y, Zhao H, Wang H N, Crawford B M, Johnson D M, Hu C, Pei C, Zhou W, Swift G B, Zhang H, Vo-Dinh T, Hu Z, Siedow J N, Pei Z M (2019) Plant cell-surface GIPC sphingolipids sense salt to trigger Ca²⁺ influx. Nature 572:341-346
Marschner P (2012) Marschner's mineral nutrition of higher plants, Ed Third edition. Academic Press
Oh D H, Leidi E, Zhang Q, Hwang S M, Li Y, Quintero F J, Jiang X, D'Urzo M P, Lee S Y, Zhao Y, Bahk J D, Bressan R A, Yun D J, Pardo J M, Bohnert H J (2009) Loss of halophytism by interference with SOS1 expression. Plant Physiol 151:210-222
Tamura K, Stecher G, Kumar S (2021) MEGA11: Molecular Evolutionary Genetics Analysis Version 11. Mol Biol Evol 38:3022-3027
Van Bel M, Silvestri F, Weitz E M, Kreft L, Botzki A, Coppens F, Vandepoele K (2022) PLAZA 5.0: extending the scope and power of comparative and functional genomics in plants. Nucleic Acids Res 50: D1468-D1474
Ye J, Coulouris G, Zaretskaya I, Cutcutache I, Rozen S, Madden T L (2012) Primer-BLAST: a tool to design target-specific primers for polymerase chain reaction. BMC Bioinformatics 13:134
Zhu J K (2016) Abiotic stress signaling and responses in plants. Cell 167:313-324

Sequence List

| | | | | Qualifier | | |
|---|---|---|---|---|---|---|
| SEQ | Sequence | | Molecule | Molecule | | |
| ID | name | Organism name | type | Type | Residues | Notes |
| 1 | AtSEA1 LP1 | Arabidopsis thaliana | DNA | other DNA | TTGACTGATGGGATGTCCTTC | Genotyping AtSEA1 |
| | | (Columbia-0 ecotype) | | | | (AT3G61770) T-DNA line |
| | | | | | | SALKseq_030394.1 |
| 2 | AtSEA1 RP1 | Arabidopsis thaliana | DNA | other DNA | TGGAGATTTGAAGCATAACGG | Genotyping AtSEA1 |
| | | (Columbia-0 ecotype) | | | | (AT3G61770) T-DNA line |
| | | | | | | SALKseq_030394.1 |
| 3 | AtSEA1 LP2 | Arabidopsis thaliana | DNA | other DNA | ATTTGATGGTGCAGGATTGTC | Genotyping AtSEA1 |
| | | (Columbia-0 ecotype) | | | | (AT3G61770) T-DNA line |
| | | | | | | SALKseq_039758.1 |
| 4 | AtSEA1 RP2 | Arabidopsis thaliana | DNA | other DNA | TGATGGTTTTGCATTGTTGAG | Genotyping AtSEA1 |
| | | (Columbia-0 ecotype) | | | | (AT3G61770) T-DNA line |
| | | | | | | SALKseq_039758.1 |
| 5 | AtSEA1 RP3 | Arabidopsis thaliana | DNA | other DNA | GCGTGCTTGAGTTTTCAATGT | Genotyping AtSEA1 |
| | | (Columbia-0 ecotype) | | | | (AT3G61770) T-DNA line |
| | | | | | | SALKseq_039758.1 |
| 6 | AtSEA1 qPCR | Arabidopsis thaliana | DNA | other DNA | CGGTATGCAAGCTGAGGTTC | qPCR primer for |
| | F1 | (Columbia-0 ecotype) | | | | Arabidopsis SEA1 |
| | | | | | | (AT3G61770) |
| 7 | AtSEA1 qPCR | Arabidopsis thaliana | DNA | other DNA | AGAACCTGCGAAGGAGTGTG | qPCR primer for |
| | R1 | (Columbia-0 ecotype) | | | | Arabidopsis SEA1 |
| | | | | | | (AT3G61770) |
| 8 | AtSEA2a qPCR | Arabidopsis thaliana | DNA | other DNA | GGAGGATTCTACAGCCACGG | qPCR primer for |
| | F1 | (Columbia-0 ecotype) | | | | Arabidopsis SEA2a |
| | | | | | | (AT1G24350) |
| 9 | AtSEA2a qPCR | Arabidopsis thaliana | DNA | other DNA | CCGTTCCCTATACCAGGAGG | qPCR primer for |
| | R1 | (Columbia-0 ecotype) | | | | Arabidopsis SEA2a |
| | | | | | | (AT1G24350) |
| 10 | AtSEA2b qPCR | Arabidopsis thaliana | DNA | other DNA | TCGCTATCGCTTTGGTTCTC | qPCR primer for |
| | F1 | (Columbia-0 ecotype) | | | | Arabidopsis SEA2b |
| | | | | | | (AT1G67600) |
| 11 | AtSEA2b qPCR | Arabidopsis thaliana | DNA | other DNA | TCCACCAGCAATAACCTGGG | qPCR primer for |
| | F1 | (Columbia-0 ecotype) | | | | Arabidopsis SEA2b |
| | | | | | | (AT1G67600) |
| 12 | AtSEA2c qPCR | Arabidopsis thaliana | DNA | other DNA | TGCCTTAGCTGTTGCCATTG | qPCR primer for |
| | F1 | (Columbia-0 ecotype) | | | | Arabidopsis SEA2c |
| | | | | | | (AT3G21610) |
| 13 | AtSEA2c qPCR | Arabidopsis thaliana | DNA | other DNA | ATTTGATTCAGCAGCTCGGC | qPCR primer for |
| | R1 | (Columbia-0 ecotype) | | | | Arabidopsis SEA2c |
| | | | | | | (AT3G21610) |
| 14 | AtSEA3 qPCR | Arabidopsis thaliana | DNA | other DNA | TATGATGCCCAGGGGGTGAG | qPCR primer for |
| | F1 | (Columbia-0 ecotype) | | | | Arabidopsis SEA3 |
| | | | | | | (AT3G12685) |
| 15 | AtSEA3 qPCR | Arabidopsis thaliana | DNA | other DNA | GCCCCTGCTATGACTTCAAC | qPCR primer for |
| | R1 | (Columbia-0 ecotype) | | | | Arabidopsis SEA3 |
| | | | | | | (AT3G12685) |
| 16 | AtSEA1 | Arabidopsis thaliana | DNA | genomic | Gcgaccgttgaatttttctcagaaaataaaaatatataagctaaagattcataatac | |
| | (AT3G61770) | (Columbia-0 ecotype) | | DNA | ccaaagtacccctattgaaaagtcaaaccttgtagcaagcccaaaggcccactttat | |
| | genomic | | | | ttgagcccagttatcctcttttggacacatttttggtatattcgagtcacgatactcgaa | |
| | sequence | | | | tggtatttccgttataatttacgaaaacaatgtctcttcttttctctacaaccacacgat | |
| | | | | | ttcgatgttaattaccaaaaaagtgtgtttaatttagctaaactaattgttttcgttgat | |
| | | | | | taggcccagttgggttgaagaaaatctccaatctttcttcgccaagtctcccacaaca | |
| | | | | | ctttgtgttctaatctctctgtatgtcgcaaagtagcagcaacttaattctctcATGG | |
| | | | | | AGTCTCTTACATCAAATCCTTCCTTCGCTTGTTTCTCTAAAACCAT | |
| | | | | | TTCTCATTACCCTTCTCGTTTCTCCTTCGTAAAGCTAACCTCTATC | |
| | | | | | CAAAAGTTGGAAGCTTCCAACAACACCTTATCGTTATTCTGCTGT | |
| | | | | | AAATCATCTTCGAATCCTAAACCAGATTGCAATCGTCGTATCAAG | |
| | | | | | TTAAACCCTTTATGTGTATTGCGTCCAATCATTCGAACAATAAAG | |
| | | | | | GGTTTGGTTTCTTCTCAATCCAGGCAATGGATGTCTCGTTTTCGA | |
| | | | | | GCTTACAGAGACGACACGGCGGCGTTTTCTGAGGATTTCGCTGG | |
| | | | | | AGATTTGAAGCATAACGGTGGATTAGGGATTGCCCTTTTGAGCG | |
| | | | | | TTACTGCTTCTGCTAAGATAAAGATTAGCCCTTTCGTTGCGACGC | |
| | | | | | TCTCTGCGAATCCAACTTTCGTTTCAGCTGTAGTCGCGTGGTTCT | |
| | | | | | TTGCGCAATCGAGTAAAATGGTTATCAATTTCTTCATTGAGAGG | |
| | | | | | AAATGGGATTTTCGTCTTCTCTATGCTTCTGGTGGGATGCCTTCG | |
| | | | | | TCTCACTCAGCTTTATGTATGGCTTTGACGACCTCTGTGGCGCTT | |
| | | | | | TGTCATGGAGTTGCAGATTCCTTGTTTCCTGTTTGTTTAGGGTTT | |
| | | | | | AGCTTGATTGTCATGTATGATGCAATTGGTGTTAGACGCCATGC | |
| | | | | | CGGTATGCAAGCTGAGgtaaaaagttaaaccaaatctgcttcaagttttgag | |
| | | | | | ttttggagatttttacagagtttgactttggtcttattaaatgctttgtctattctacaag | |
| | | | | | ttgactagattttcatgttgatgagtgtgtatagtttaatgtattactatttccatgttga | |
| | | | | | tgagtgtgtatagtttaatgtattactagaatctggtgatggattcgactttcactacat | |
| | | | | | tgttgaatttttgtgaatgcttttatgtgtaaaagttggcgtgcttgagttttcaatgtgt | |
| | | | | | gcacaattgaagtacatcactagatttggaattatggagagttagatgttgatttctgt | |
| | | | | | tcttggttcttgaatcttttatatgatggttttgcattgttgagagcgtaatacaagtttc | |
| | | | | | tcgaaattatggacattgggcacatgttgatgatagctgttattggttgtagaatgac | |
| | | | | | cttgataaaattgcagagaattaagagttttgaaagaggattcacacagagtctaag | |
| | | | | | tcatgtttgctcttggttcttaaatcttttagatgatggttttgcattgttgagagcacaa | |
| | | | | | tacaagtttctcgaaattatggatattagccacatattgatgatagttgttgttggttgt | |
| | | | | | agaatgatcttgataaaattgcagagaattaagagttttgaaagagggtctgtacag | |
| | | | | | agtctaagtctagtttgttgttctttgtaagataagattaacaatgttttgtatttgttga | |
| | | | | | tgtcagGTTCTGAACTTGATCATTAGGGACTTGTTCGAAGGACATC | |
| | | | | | CCATCAGTCAAAGAAAGCTTAAGGAACTGCTTGGTCACACTCCT | |
| | | | | | TCGCAGGTTCTTGCTGGAGCATTGGTTGGTATTGTGATTGCTTG | |
| | | | | | CTTTTGTTGCCAAGGCTATCTCGTCTCAGTCTAAgaagatgtcttttat | |
| | | | | | tccaagtgatctggttggtttttgatttgggtagcctaaaaaaacagagtgttttaagt | |
| | | | | | atcgcttaacattgattttggctaagattcatgttgtagtatcatatgttttgttcagca | |
| | | | | | atagagaaaactcttgtttcttcttccttgtggcttttatttttttgagtttttgtattccct | |
| | | | | | gagaatgcaatgagagcttcttttgtttaaagcgtttaaagcaatgcatgacatgttc | |
| | | | | | agtgactgctgcagaaacacacacacacacacacacacaaagttaaccataataac | |
| | | | | | taagattta | |
| 17 | AtSEA2a | Arabidopsis thaliana | DNA | genomic | Atgaacatcacaattttattccacaaattcctttgtatactacttattttcaaataaaat | |
| | (AT1G24350) | (Columbia-0 ecotype) | | DNA | cacgttatttttgtaatggtgtttcacgatttctttacatatggtaatggtaatgttaaa | |
| | genomic | | | | caagaataaatatatttttaactcggattcggttgtaaatctgttttggtactctccagt | |
| | sequence | | | | ggcttcaataggcaatgaagcagaagagttgtgtctatgcatgttcttgacgTTAG | |
| | | | | | CTTGTAGCTATCCTGAAAAATAAGTATCCGGTGACTGCTGTAGC | |
| | | | | | ACTTCCAAGCATTCCACCAGCAACGACctgaattctcagcaaatggaaa | |
| | | | | | acagaatctgatgagaactagaaattcattgatatcaacacggttacaaatcaaag | |
| | | | | | agtagttcatttatcaaccaaagtaaacacaaactagggtctgttctagagttccatc | |
| | | | | | agctcacacaagcctccaaaagaccctcaactctgttcccaattacacgatccccctc | |
| | | | | | acCGATTCCTTCAGCACTCTATTTGTACTCATGATTTCCCCTAACT | |
| | | | | | CTGTAACTATcttgctaaatgtaacagctaaaagaatctcatatcttgctagtcat | |
| | | | | | atatgaaaagactttttcaagtcatatgtctacatacCTGGGGAGGGGTATG | |
| | | | | | ACCAAGAAGTTCCCGCAAAGGTCTGCTTTCAGCCAGCGGATGTT | |
| | | | | | CTGCAGGAAGTTCATATACAATCTGATTGAGAACctgaaaaatgaat | |
| | | | | | cagaccccagagatcaggataccagagctcaaatgcatcatgacaaaaccaatagt | |
| | | | | | ggttcttttatcaaaccagtcaagttagaagatgaaacaaaggaaaggttagagca | |
| | | | | | gcaatgaacaaacCTCAGCTTGACGACCAGCATGTAATCTAACACCG | |
| | | | | | GTAGCATCATACATCACctaccaacccaaaccactcacatattaatcgtcact | |
| | | | | | tgattccttaatagccaatttgattaaactagagaaagtatccaacttacAACAGA | |
| | | | | | TGCCAAAATCAAAGCAATGGCAAAATGTGATCCACCAAAGCCTT | |
| | | | | | CTTGTAAACCAATAGCTACAGCGAGAGCTGTAACGGTCGCTGAA | |
| | | | | | TGCGAAGAAGGCATTCCTCCAGACCCAATAAGCTGTTTGAGATC | |
| | | | | | CCATCTCCGTTCCCTATACctacagaatctaggaaaccatgtaaagatccttt | |
| | | | | | ctcatctaacgtatactataatttcctatgtaccgtttcgaaccaaagattgaggaac | |
| | | | | | caggcacatatagatttaatcggagtctgatccaaacagagatataaagagagaac | |
| | | | | | gtacCAGGAGGTGAAGAGTTTGATGAATTGAGCGATAGTGAAAG | |
| | | | | | AGGTGACGGCGGAAATGAGAGGATAATTCGTGAAAATGGAGA | |
| | | | | | AATAATGAGTGGAAGACGAAGACGTAGCCGTGGCTGTAGAATC | |
| | | | | | CTCCATggccgatgatggatctagtcaggctttaaatcgtgctagcgaaagaaga | |
| | | | | | agaagaaaatgacaccgaaacgacaatgatccataaaacgtcgactaattcccttt | |
| | | | | | caaactccaatggcgtctacgtctaccgttcaatcctctttgtctttctttttttttctttg | |
| | | | | | accaattttttttttaaacactgctccacaaaactatatattatacaattacccgcatcc | |
| | | | | | gaacaaatatgaaccgtatccgatacaaaacccgaatacctaggtcagttctattcg | |
| | | | | | tgaaatcgagtctcctcttattcgacgacctgccccaactataaacatgacttttacac | |
| | | | | | acttgacataacttcgtttgtatcgcttttttgaaattctatatattattattgtttgagc | |
| | | | | | agaaactaagacaaaattaagacttttctctcttcttcttctccactgggtcgatcttta | |
| | | | | | cgaggttcacttgtacggattcactttaacaacagattcgccA | |
| 18 | AtSEA2b | Arabidopsis thaliana | DNA | genomic | tatcacacattagaggaattttatcagctagcttgtgccagaggaaacagaacttaa | |
| | (AT1G67600) | (Columbia-0 ecotype) | | DNA | tgctaatatatatgtgtttaatgagccaaaagaaataaagcaaatcaacattagctc | |
| | genomic | | | | tataagcaaaactttgtagtcatggaatttaagaggaaatgagatgatgagttttaa | |
| | sequence | | | | agtattgatgctcctgagtgctgaatctaaacagatcatgttcgttgtagttcaatgca | |
| | | | | | caagctcccattttaccactttacagatctgtgtaatgacgtttcaagtttcctttacta | |
| | | | | | gagcaaacctaactaaaatatattgtcaatgacaagaacagagcagcaaaaaaaa | |
| | | | | | cagagaaattaagctcaatttccaaattctatttgcatgtcatggctatctttaaacta | |
| | | | | | cctagcagcgccttgacacacaatggagcagaaaaggcgagattttgctgttcccag | |
| | | | | | cttaagCTACTTGGCTAACAAGATGACTAAGTACCCAACAACTGCT | |
| | | | | | GTAGATATTCCAAGCATTCCACCAGCAATAACctgacgccaccaaaat | |
| | | | | | atagatagtctcaagtactaaccacaatcattctcttgcttggtgtgatctgacacaat | |
| | | | | | tagtactacatacttacCTGGGGAGGAGTATGACCTAGAAGTTCACGC | |
| | | | | | AAAGGTCTGGTTTCAGCCAGAGGATGCTCTGCAGGAAGTTCATA | |
| | | | | | CACAATCTGATTTAGAACCTCAGCTTGGCGTCCAGCATGTAACCT | |
| | | | | | TACACCAGTTGCATCATACATAACctaccaaaccaaacaatcttaaattct | |
| | | | | | taatcatcccctgaagtgatataatcaagaaaggtcattaattaattaagtgattagc | |
| | | | | | taaaaacatacAATGGTAGTGAGAACCAAAGCGATAGCGAAATGT | |
| | | | | | GAACCACCAAAACCTTCTTGTAAACCAACAGCTAAAGCTAAAGC | |
| | | | | | TGTAACCGTTGCTGAATGTGATGAAGGCATACCTCCAGACCCAA | |
| | | | | | CAAGCCGTTTCAAATCCCATCTCCTTTCTTTATACctacaccaagtagt | |
| | | | | | tgtaaaatcaaatttctacaactagtctgtttctttaacctagacagatatgaaaacgt | |
| | | | | | acCAGGAGGTGAAGAATTTGATGAATTGTGCGATGGTGAAAGC | |
| | | | | | TAAAACGGCGGAAATCAGAGGGTAATTTGTGAAAATTGAGATA | |
| | | | | | TAGTGCGAGGAAGATGAAGCCACCGATTCCTCCATtgaatgatggtg | |
| | | | | | atcgattgatctcgtatcgtagggctttaatcgtctgatagctcttctacaggataaag | |
| | | | | | aacatacagagaagacataaagttgcgtgtagaaatctttaccttgagactcctcta | |
| | | | | | atggcttcttcttcctcaaattttattttcctagttttctcgctttttcttcggcaacaaac | |
| | | | | | aacagcttttatttaatgttttttactaacaagtctacacgacctaagtacagatgaga | |
| | | | | | ttattacttttttttatcaccgtttatattataatgacaaaacagctacatatcaataaa | |
| | | | | | gaattgacatctgaaaaattgcatattatctgaaattatataatagaaatattatttta | |
| | | | | | taaactctgttgatatgttagaagtttttgcgatattaatatacgtatagagctttttat | |
| | | | | | tgtttaaagtttttatccagaaagaaagtttaaattctgctagttggttcaaaaatgca | |
| | | | | | cattcacaaaaatatagcatgcgatattagaatactgaatacactatcaatctatgat | |
| | | | | | tgatcagattatcagaatatgcttctgagcttctctgtttgctagagaatatgcttttta | |
| | | | | | ggctttaagtgaaaaacaacT | |
| 19 | AtSEA2c | Arabidopsis thaliana | DNA | genomic | tctgattctacaattgccatgccagtcattaacgaataatatccaatacagatcttcg | |
| | (AT3G21610) | (Columbia-0 ecotype) | | DNA | aattagttaagctgactcaaaagtttcactagcttcacattttctatcaataatggtat | |
| | genomic | | | | caataatgtgcttttaggccataaaacttcttgtagtctagtggtcatagatatggttt | |
| | sequence | | | | cgcataaaactgatcatgattaggaggaagaggaaatagagagattatatatatat | |
| | | | | | atatagagagagagagaatgttgagaaggcgtaagcttaaacaatgacaagtcta | |
| | | | | | actaagatcatctatatattacacagatttaacaatttccatttactccaataatctag | |
| | | | | | cgtcacttgatatcgctgaatctatcttagtacctgaagggtctatgatttcaaaaga | |
| | | | | | attcgggatttgttcttgtgtagagagactgaCTAGCTTGAGCTCCTCATTAA | |
| | | | | | GTAAGCTACCACGCATCCTAAAATCCCACCTGCTGCAACctgtattt | |
| | | | | | caatctcataacttagcaatagaatgtaacatcctatggttaatacatggcgaatacg | |
| | | | | | gtgactttagttctatatgtttcagctttatgaaatctagatctaattctaacatggttat | |
| | | | | | gacacagtcattggacttaaaaatgtttaatgattggaatgataaaaagaattgact | |
| | | | | | ggtgggtatagaaaacCTGGATAGGAGTGTGGCCAAGCAGTTCACGC | |
| | | | | | AATGGTCTAACTGTTGATAATGGATGCTCCGGAGGAAACTCACA | |
| | | | | | AACAATTTGATTCAGCAGctgaaatttccaatcaagtgaaaaaggcttgcgg | |
| | | | | | tttcagaaagcatggaacaataaaagaagatgaaaacagatgagattaataacttt | |
| | | | | | tctatagctttacCTCGGCTTGACGACCAGCATGAAGCCTGACACCA | |
| | | | | | GAGGCATCATACATAACctgcagcccaattcagaaatatacaaaagattggt | |
| | | | | | cttactatgaaaccaaataggctattcacaatgaaagcaaaaatctcttttctacact | |
| | | | | | aaacaaagcaaggcatttgtaagacaaattgtgaagtggaaaagaagatgagaag | |
| | | | | | aacactcacAACGCAAGCCAAGACAACTGCAATAGCAAAAGCTGG | |
| | | | | | TGCTCCAGCTCCTTCTTCAAAGCCAATGGCAACAGCTAAGGCAG | |
| | | | | | TGACAGTAGCAGAATGAGAAGAAGGCATTCCACCAGAACTAAT | |
| | | | | | CATACGTTTAGAGTCCCACCTCTTTTCTTTGTACctgaacatctaacat | |
| | | | | | acatcacgaaacaactgagaattccacttctttcaatcttaactacaacagatctata | |
| | | | | | gctacacaaatgccaacattcaacttctgagacctaatcatgagcaaaaatttgcaa | |
| | | | | | atcctaattaatccatcgaaaatcgaaaccctaacagtaagaaatttgttcaatcac | |
| | | | | | agatcttaagcagagatcaaactaatctgagaaaacgaaagatttgaacagacCA | |
| | | | | | ATTAGTGAAGACTTTGAGGAACTGAGCAAGAGCGAAGGCAAGA | |
| | | | | | AAAGCGGAGAAGATTGGGAGATTGTGAGGAAACAGATTATGC | |
| | | | | | GACGGAGGAGATCCGTACAACGCCCTATTGCCGCCACCTAACGA | |
| | | | | | ACCAACGTCCGCCGCTGTCATCACCTCGTCCATaggcgatatacaacc | |
| | | | | | gcgaaaaaatcccgatctcttcttcttcttcgatcaaaatcagacactgcgaggatca | |
| | | | | | atgtcaacgtaaaaaggtaaagagattcaggagaagagagtattgaattgttttcga | |
| | | | | | taagcttttaggctttttgtgttggttccgtatttgggaaggatcaattattgtgttaaa | |
| | | | | | aggaaatcaattttacctttttaatttttttgcacttttgtttttcaatattgctgaatgtt | |
| | | | | | gactaataacttatttgatttgttttagataaatttgatgacctctatttgatattgttttt | |
| | | | | | tttttgtatatatatgatatagcaaaaaccaaagtaaactttg | |
| 20 | AtSEA3 | Arabidopsis thaliana | DNA | genomic | taaattctctttctttcttatcaacacaaaaaatatatagtttttataaagttgggagct | |
| | (AT3G12685) | (Columbia-0 ecotype) | | DNA | tttTTACATCAGGGAATAAACACTGAGGGTAACTAAGAAACCGA | |
| | genomic | | | | ACAAAGCCCCTGCTATGACTTCAACTTCAGTGTGTCCTATTGATT | |
| | sequence | | | | CTTTCAATGGAGGAGCAATCTCCTCACTGATCTCTTCTGATGTCA | |
| | | | | | AAGCTTTGTTAGATTCGTTGCCCTTTAAACTCATTACTTCACTTCT | |
| | | | | | ACGAGCATTAGCTGTTAGTTTGTTCAACACTTTGGCATGTTTTCC | |
| | | | | | AACTTCTCTCCTCACCCCctgcaatgttacaatcagtgagacattaagataac | |
| | | | | | actagaatcagacattgagaatttgagaataaccaacaaagacctattgaacaaaa | |
| | | | | | gccagcctaagcttttctctatagactattttacCTGGGCATCATACATGATA | |
| | | | | | AGGCCAGCATAAACCACAGTTAGACCAAAAATGGAGTCAGCAA | |
| | | | | | ATCCCctgaagccaaaccatattcaggaaattgcataagctgagtatatttagggg | |
| | | | | | aattgagaagagaaaggttcaggctaatcacCTTTCAAAAGCAATAGCAGT | |
| | | | | | TGCTGCAGCCACAACAGActggtttctcaagaattaggccaaaatgtcagag | |
| | | | | | aagtagacaaacaatattaccagaggaaacaagaataaaaaaaggataagcttac | |
| | | | | | AGAAGAATGTGTTGAAGGGAAACCTCCAGCTTGAAAGACACTT | |
| | | | | | CTAAAGTCCAGATTTTTCCCATACAAAACGACAGAAGTGAATGG | |
| | | | | | CTTTGAGAGCTGCCCAATCACAGCAGAAGTCCCAGCAGCTATGA | |
| | | | | | GTACcttcacgccggtgaaatcaaaagcctaagtgacgatgaccctccaaaaaaa | |
| | | | | | ggaatgaaagaagagaaaaaggcgaaacCTTGTTGTGGATGACCTCAGC | |
| | | | | | GATATCTTGGAATCCAACATTGACAACGCAGGTGAGACGAGGT | |
| | | | | | GGCTTCTTTGGGCAGCTTGCAAGATTGAATTTGGAGTTGGAGAA | |
| | | | | | AGAGAAACAGAGATCATTGGCGTTATGAGACACAAAACAGTGC | |
| | | | | | TTCATCAACATctctgtgcagaagatggaactttggtcggaggatgacgataag | |
| | | | | | aataaggcagcgagaaatttggaaccaatggttctgacgctctgttatgtcgagaag | |
| | | | | | agtgttgtctcttaggacattttaattgactcagagaagcttttgtggattttttcattg | |
| | | | | | acaaattgttatctcacacacaaaatctgctttaactcttttagtaaaactcttttagtc | |
| | | | | | tcttaaaccaagtcatctgttcttcttttataaatcacaatttgtattcgccaaaattaa | |
| | | | | | agaatatgaacaaaaacaaacatatcgaaactttcagcttgtggagttttaagtC | |
| 21 | AtSEA1 CDS | Arabidopsis thaliana | DNA | genomic | ATGGAGTCTCTTACATCAAATCCTTCCTTCGCTTGTTTCTCTAAAA | |
| | | (Columbia-0 ecotype) | | DNA | CCATTTCTCATTACCCTTCTCGTTTCTCCTTCGTAAAGCTAACCTC | |
| | | | | | TATCCAAAAGTTGGAAGCTTCCAACAACACCTTATCGTTATTCTG | |
| | | | | | CTGTAAATCATCTTCGAATCCTAAACCAGATTGCAATCGTCGTAT | |
| | | | | | CAAGTTAAACCCTTTATGTGTATTGCGTCCAATCATTCGAACAAT | |
| | | | | | AAAGGGTTTGGTTTCTTCTCAATCCAGGCAATGGATGTCTCGTTT | |
| | | | | | TCGAGCTTACAGAGACGACACGGCGGCGTTTTCTGAGGATTTCG | |
| | | | | | CTGGAGATTTGAAGCATAACGGTGGATTAGGGATTGCCCTTTTG | |
| | | | | | AGCGTTACTGCTTCTGCTAAGATAAAGATTAGCCCTTTCGTTGCG | |
| | | | | | ACGCTCTCTGCGAATCCAACTTTCGTTTCAGCTGTAGTCGCGTGG | |
| | | | | | TTCTTTGCGCAATCGAGTAAAATGGTTATCAATTTCTTCATTGAG | |
| | | | | | AGGAAATGGGATTTTCGTCTTCTCTATGCTTCTGGTGGGATGCCT | |
| | | | | | TCGTCTCACTCAGCTTTATGTATGGCTTTGACGACCTCTGTGGCG | |
| | | | | | CTTTGTCATGGAGTTGCAGATTCCTTGTTTCCTGTTTGTTTAGGG | |
| | | | | | TTTAGCTTGATTGTCATGTATGATGCAATTGGTGTTAGACGCCAT | |
| | | | | | GCCGGTATGCAAGCTGAGGTTCTGAACTTGATCATTAGGGACTT | |
| | | | | | GTTCGAAGGACATCCCATCAGTCAAAGAAAGCTTAAGGAACTGC | |
| | | | | | TTGGTCACACTCCTTCGCAGGTTCTTGCTGGAGCATTGGTTGGTA | |
| | | | | | TTGTGATTGCTTGCTTTTGTTGCCAAGGCTATCTCGTCTCAGTCT | |
| | | | | | AA | |
| 22 | AtSEA2a CDS | Arabidopsis thaliana | DNA | genomic | ATGGAGGATTCTACAGCCACGGCTACGTCTTCGTCTTCCACTCAT | |
| | | (Columbia-0 ecotype) | | DNA | TATTTCTCCATTTTCACGAATTATCCTCTCATTTCCGCCGTCACCT | |
| | | | | | CTTTCACTATCGCTCAATTCATCAAACTCTTCACCTCCTGGTATAG | |
| | | | | | GGAACGGAGATGGGATCTCAAACAGCTTATTGGGTCTGGAGGA | |
| | | | | | ATGCCTTCTTCGCATTCAGCGACCGTTACAGCTCTCGCTGTAGCT | |
| | | | | | ATTGGTTTACAAGAAGGCTTTGGTGGATCACATTTTGCCATTGCT | |
| | | | | | TTGATTTTGGCATCTGTTGTGATGTATGATGCTACCGGTGTTAGA | |
| | | | | | TTACATGCTGGTCGTCAAGCTGAGGTTCTCAATCAGATTGTATAT | |
| | | | | | GAACTTCCTGCAGAACATCCGCTGGCTGAAAGCAGACCTTTGCG | |
| | | | | | GGAACTTCTTGGTCATACCCCTCCCCAGGTCGTTGCTGGTGGAA | |
| | | | | | TGCTTGGAAGTGCTACAGCAGTCACCGGATACTTATTTTTCAGG | |
| | | | | | ATAGCTACAAGCTAA | |
| 23 | AtSEA2b CDS | Arabidopsis thaliana | DNA | genomic | ATGGAGGAATCGGTGGCTTCATCTTCCTCGCACTATATCTCAATT | |
| | | (Columbia-0 ecotype) | | DNA | TTCACAAATTACCCTCTGATTTCCGCCGTTTTAGCTTTCACCATCG | |
| | | | | | CACAATTCATCAAATTCTTCACCTCCTGGTATAAAGAAAGGAGAT | |
| | | | | | GGGATTTGAAACGGCTTGTTGGGTCTGGAGGTATGCCTTCATCA | |
| | | | | | CATTCAGCAACGGTTACAGCTTTAGCTTTAGCTGTTGGTTTACAA | |
| | | | | | GAAGGTTTTGGTGGTTCACATTTCGCTATCGCTTTGGTTCTCACT | |
| | | | | | ACCATTGTTATGTATGATGCAACTGGTGTAAGGTTACATGCTGG | |
| | | | | | ACGCCAAGCTGAGGTTCTAAATCAGATTGTGTATGAACTTCCTG | |
| | | | | | CAGAGCATCCTCTGGCTGAAACCAGACCTTTGCGTGAACTTCTA | |
| | | | | | GGTCATACTCCTCCCCAGGTTATTGCTGGTGGAATGCTTGGAAT | |
| | | | | | ATCTACAGCAGTTGTTGGGTACTTAGTCATCTTGTTAGCCAAGTA | |
| | | | | | G | |
| 24 | AtSEA2c CDS | Arabidopsis thaliana | DNA | genomic | ATGGACGAGGTGATGACAGCGGCGGACGTTGGTTCGTTAGGTG | |
| | | (Columbia-0 ecotype) | | DNA | GCGGCAATAGGGCGTTGTACGGATCTCCTCCGTCGCATAATCTG | |
| | | | | | TTTCCTCACAATCTCCCAATCTTCTCCGCTTTTCTTGCCTTCGCTCT | |
| | | | | | TGCTCAGTTCCTCAAAGTCTTCACTAATTGGTACAAAGAAAAGA | |
| | | | | | GGTGGGACTCTAAACGTATGATTAGTTCTGGTGGAATGCCTTCT | |
| | | | | | TCTCATTCTGCTACTGTCACTGCCTTAGCTGTTGCCATTGGCTTTG | |
| | | | | | AAGAAGGAGCTGGAGCACCAGCTTTTGCTATTGCAGTTGTCTTG | |
| | | | | | GCTTGCGTTGTTATGTATGATGCCTCTGGTGTCAGGCTTCATGCT | |
| | | | | | GGTCGTCAAGCCGAGCTGCTGAATCAAATTGTTTGTGAGTTTCC | |
| | | | | | TCCGGAGCATCCATTATCAACAGTTAGACCATTGCGTGAACTGC | |
| | | | | | TTGGCCACACTCCTATCCAGGTTGCAGCAGGTGGGATTTTAGGA | |
| | | | | | TGCGTGGTAGCTTACTTAATGAGGAGCTCAAGCTAG | |
| 25 | AtSEA3 CDS | Arabidopsis thaliana | DNA | genomic | ATGTTGATGAAGCACTGTTTTGTGTCTCATAACGCCAATGATCTC | |
| | | (Columbia-0 ecotype) | | DNA | TGTTTCTCTTTCTCCAACTCCAAATTCAATCTTGCAAGCTGCCCAA | |
| | | | | | AGAAGCCACCTCGTCTCACCTGCGTTGTCAATGTTGGATTCCAA | |
| | | | | | GATATCGCTGAGGTCATCCACAACAAGGTACTCATAGCTGCTGG | |
| | | | | | GACTTCTGCTGTGATTGGGCAGCTCTCAAAGCCATTCACTTCTGT | |
| | | | | | CGTTTTGTATGGGAAAAATCTGGACTTTAGAAGTGTCTTTCAAG | |
| | | | | | CTGGAGGTTTCCCTTCAACACATTCTTCTTCTGTTGTGGCTGCAG | |
| | | | | | CAACTGCTATTGCTTTTGAAAGGGGATTTGCTGACTCCATTTTTG | |
| | | | | | GTCTAACTGTGGTTTATGCTGGCCTTATCATGTATGATGCCCAGG | |
| | | | | | GGGTGAGGAGAGAAGTTGGAAAACATGCCAAAGTGTTGAACA | |
| | | | | | AACTAACAGCTAATGCTCGTAGAAGTGAAGTAATGAGTTTAAAG | |
| | | | | | GGCAACGAATCTAACAAAGCTTTGACATCAGAAGAGATCAGTG | |
| | | | | | AGGAGATTGCTCCTCCATTGAAAGAATCAATAGGACACACTGAA | |
| | | | | | GTTGAAGTCATAGCAGGGGCTTTGTTCGGTTTCTTAGTTACCCTC | |
| | | | | | AGTGTTTATTCCCTGATG | |
| 26 | AtSEA1 | Arabidopsis thaliana | AA | protein | MESLTSNPSFACFSKTISHYPSRFSFVKLTSIQKLEASNNTLSLFCCKS | |
| | (AT3G61770) | (Columbia-0 ecotype) | | | SSNPKPDCNRRIKLNPLCVLRPIIRTIKGLVSSQSRQWMSRFRAYRD | |
| | protein | | | | DTAAFSEDFAGDLKHNGGLGIALLSVTASAKIKISPFVATLSANPTFV | |
| | | | | | SAVVAWFFAQSSKMVINFFIERKWDFRLLYASGGMPSSHSALCMA | |
| | | | | | LTTSVALCHGVADSLFPVCLGFSLIVMYDAIGVRRHAGMQAEVLNL | |
| | | | | | IIRDLFEGHPISQRKLKELLGHTPSQVLAGALVGIVIACFCCQGYLVS | |
| | | | | | V | |
| 27 | AtSEA2a | Arabidopsis thaliana | AA | protein | MEDSTATATSSSSTHYFSIFTNYPLISAVTSFTIAQFIKLFTSWYRERR | |
| | (AT1G24350) | (Columbia-0 ecotype) | | | WDLKQLIGSGGMPSSHSATVTALAVAIGLQEGFGGSHFAIALILAS | |
| | protein | | | | VVMYDATGVRLHAGRQAEVLNQIVYELPAEHPLAESRPLRELLGHT | |
| | | | | | PPQVVAGGMLGSATAVTGYLFFRIATS | |
| 28 | AtSEA2b | Arabidopsis thaliana | AA | protein | MEESVASSSSHYISIFTNYPLISAVLAFTIAQFIKFFTSWYKERRWDLK | |
| | (AT1G67600) | (Columbia-0 ecotype) | | | RLVGSGGMPSSHSATVTALALAVGLQEGFGGSHFAIALVLTTIVMY | |
| | protein | | | | DATGVRLHAGRQAEVLNQIVYELPAEHPLAETRPLRELLGHTPPQVI | |
| | | | | | AGGMLGISTAVVGYLVILLAK | |
| 29 | AtSEA2c | Arabidopsis thaliana | AA | protein | MDEVMTAADVGSLGGGNRALYGSPPSHNLFPHNLPIFSAFLAFAL | |
| | (AT3G21610) | (Columbia-0 ecotype) | | | AQFLKVFTNWYKEKRWDSKRMISSGGMPSSHSATVTALAVAIGFE | |
| | protein | | | | EGAGAPAFAIAVVLACVVMYDASGVRLHAGRQAELLNQIVCEFPP | |
| | | | | | EHPLSTVRPLRELLGHTPIQVAAGGILGCVVAYLMRSSS | |
| 30 | AtSEA3 | Arabidopsis thaliana | AA | protein | MLMKHCFVSHNANDLCFSFSNSKFNLASCPKKPPRLTCVVNVGFQ | |
| | (AT3G12685) | (Columbia-0 ecotype) | | | DIAEVIHNKVLIAAGTSAVIGQLSKPFTSVVLYGKNLDFRSVFQAGG | |
| | protein | | | | FPSTHSSSVVAAATAIAFERGFADSIFGLTVVYAGLIMYDAQGVRRE | |
| | | | | | VGKHAKVLNKLTANARRSEVMSLKGNESNKALTSEEISEEIAPPLKE | |
| | | | | | SIGHTEVEVIAGALFGFLVTLSVYSLM | |
| 31 | ATR0317G115 | Amborella trichopoda | AA | protein | MDEVLVAGDGRPFFFNNNNNNTTTPSSSSSPVSLSNLPLLSSFLAF | |
| | | | | | AIAQFLKLFTTRYKEKRWDAKRLLGSGGM7PSSHAATVTALAIAIGLR | |
| | | | | | EGAGSSMFALAVVVASVVMYDASGVRLHAGRQAELLNQIVCELPP | |
| | | | | | EHPLSTVRPLREPLGHTPVQVVVGAVLGCMVAYLMRETPNNIN | |
| 32 | ATR0602G097 | Amborella trichopoda | AA | protein | MDFPSLSPFCVLIPVLHRITDYFTVKGFHGGKTLRTTMVKWVSGVK | |
| | | | | | NGGRTGACLDDDLLNSGGIGMALLSTTAMAKGHISPVMATLSAN | |
| | | | | | PTFMSGLFAWVIAQSTKVLLNFFVERKWDFQMLVSSGGMPSSHS | |
| | | | | | ALCTALTTSVALCHGISDALFPVCLGFSLIVMYDATGVRRHAGMQA | |
| | | | | | EVLNMIIEDLFQGHPVSQRKLKELLGHTPSQVIAGAFLGILVACMCC | |
| | | | | | QGRIRVAMGLVARHGLSPNVKTAGSVQGGLTASDCRLCLTAPYG | |
| 33 | ATR0737G104 | Amborella trichopoda | AA | protein | MLSSTANFSSILAPKVLSSTAHKRPFVCLKFGIEEIKEISQNKVLVSAT | |
| | | | | | VAAAIGQFSKPFTSAIYGKGFDFAAAARSGGMPSTHSAGVVAAAA | |
| | | | | | AIGLERGFSDSIFGLAVVLAAIVVYDSQGVRKEVGTHAKILNNTILKS | |
| | | | | | EAQHGFLETENGDLADEDLEAASVEAGATISSLFSSTNDFYTKDEKR | |
| | | | | | AKLNSNYSQINKPSDVLRKSLKMPLIEEAGKEAKKIGNRYPRLKESV | |
| | | | | | GHTEAEVFVGILLGFLVTWAVYSVM | |
| 34 | ATR0824G048 | Amborella trichopoda | AA | protein | MPSSHSATVTALAVAIGFKDGFEGSVFAVAMVFACVVMYDAFGV | |
| | | | | | RLHAGRQAEVLNQIVYELPAEHPLADTRPLRELLGHTPTQVIAGAM | |
| | | | | | LGCVTAIVARILSLL | |
| 35 | Cre02.g106350 | Chlamydomonas | AA | protein | MRPVFLEGPGTEVQYPVSAHTANYSDALTAREVILHTGFVGLFFNG | |
| | | reinhardtii | | | CLVSAFIAFFIAQTCKVFTHYYTEQVWDLQRMVGSGGMPSSHTALI | |
| | | | | | VALTTAVGVENGTSSTLFAACLVLALIVMYDATGVRLHAGRQATVL | |
| | | | | | NIIIAEMPPDHPVQDGGRLRDSLGHTPIQVAVGAVLGVVVGLVVE | |
| | | | | | NLYLLGDKSGAGGSFH | |
| 36 | Cre12.g530100 | Chlamydomonas | AA | protein | MRRQFDSAILADNSSMAQPGLAVPLLAAGLAIPPVPGNGVADLLT | |
| | | reinhardtii | | | NRVFLVGFWSWFSAQFLKIFTKRFKKGVWDLGAMLESGGMPSSH | |
| | | | | | SSLCAGITTAIAIQQGLGSPLFAACLCFSVIVMYDAMGVRRHAGKQ | |
| | | | | | AEVLNKVIDELLDDDHPMGEVKLKEVLGHTPRQVVCGGLLGLAVG | |
| | | | | | LFFPAC | |
| 37 | Medtr2g062820 | Medicago truncatula | AA | protein | MMLLQQSNFCPIPSSQFPSLKQRNPFLHHLQFRRKDKASTFRISSLA | |
| | | | | | AAGFFNDVAQIAHNKVLIAAGVSMAIGQLSKPFTSVFLYGKEFDIKA | |
| | | | | | LIQAGGFPSSHSSATVACATLLGLERGLSDPIFGLAVVYAGLIMYDA | |
| | | | | | QGVRREVGIHARTINKLLLQMHVNHLHSKHKDGLINSQPGSSSPPK | |
| | | | | | AETQEKSLLFQETTSLEPQQANTNVLVKSESIIRQSDEELQSSDFLED | |
| | | | | | AKETSKLVADGLLPLKESVGHTEVEVVAGGLLGFLVGLAVYNLK | |
| 38 | Medtr2g087870 | Medicago truncatula | AA | protein | MNEVLTRADVTASTASSLSPFVPSSNLPLISAFLSFALAQFLKIFTTW | |
| | | | | | YKEKRWDSKRLLDSGGMPSSHSATVSALAVAIGFQEGIGSSVFAIA | |
| | | | | | VILACIVMYDATGVRLHAGRQAELLNQIVCELPPEHPLSNVRPLRDS | |
| | | | | | LGHTPLQVVAGGLLGCIIAFLMRKSS | |
| 39 | Medtr3g085670 | Medicago truncatula | AA | protein | MNETAPTTSSSSSIFHNYPLISAILAFTIAQSIKFFTVWYKEKRWDPK | |
| | | | | | QLVGSGGMPSSHSATVTALATAVGFHEGFGGPLFATALVMAIIVM | |
| | | | | | YDATGVRLQAGRQAEVLNQIVIELPAEHPLSDSRPLRELLGHTPPQ | |
| | | | | | VIAGSLLGFITSSIGYIITMFGS | |
| 40 | Medtr7g117380 | Medicago truncatula | AA | protein | MEYSLLPLTPKLPSSSFHFNTLRPISTSCSQSNSNNTTSTNSYNSQRN | |
| | | | | | PTWVSFPFTQTIKHLTALNTHNKVSNENCCYLONGGLGIALLSVTA | |
| | | | | | NAKVKISPFVATLASNPTFVSGLFAWFLAQSIKFFLNFFVERKWDLS | |
| | | | | | LFCASGGMPSSHSALCTALTTSVAICHGVADSLFPVSLGFSLIVMYD | |
| | | | | | AIGVRRHAGMQAQVLNMILADMFQGHPISERKLKELLGHTPSQVF | |
| | | | | | AGALLGFLVACFCCQGCVVVG | |
| 41 | Os01g0901800 | Oryza sativa ssp. | AA | protein | MGDDGGGDGGASSAGFSYFAVFHNYPLVAALLGFAVAQSIKFFVT | |
| | | japonica | | | RYKENRWDPKQLIGSGGMPSSHSATVTALAVAIGFQDGFGCALFA | |
| | | | | | TAAIFASVVMYDASGIRLHAGKQAEVLNQIVCELPSEHPLSETRPLR | |
| | | | | | ELLGHTPTQVVAGALLGSMLATAGQMFLVVSGSV | |
| 42 | Os04g0486900 | Oryza sativa ssp. | AA | protein | MAAAAAVVNYPLVAALVAFALAQSSKFFTTWFKEKRWDARQLIAS | |
| | | japonica | | | GGMPSSHSATVTALAVAIGIQEGYRSATFATSVIIACVVMHDAFGV | |
| | | | | | RLHAGKQAEVLNQIVYELPEEHPLSETKPLREILGHTVPQVVAGCIIG | |
| | | | | | ILIAVVMRLALWSS | |
| 43 | Os05g0534100 | Oryza sativa ssp. | AA | protein | MEVLSSSRFSRVFPSSLLSPVSKPLKPPRHRRRRVQTLSSSSSDAAAA | |
| | | japonica | | | PSTPPPVWPPASLSRLLAAALRGGRAGGELPDLAVGAGAAATAAG | |
| | | | | | GGARIGTLLMSTTAAAVTKARENPYILALAANPTFVSGLVAWAVA | |
| | | | | | QAAKVVLTSFVERRWDLRMLFSSGGMPSSHTALCTALTASVALCH | |
| | | | | | GVSDSLFPVCLGFTLIVMYDATGVRRHAGMQAEVSQCFDCFCPFL | |
| | | | | | YCI | |
| 44 | Os05g0548800 | Oryza sativa ssp. | AA | protein | DYIFFLQVMYDASGIRFHTGRQAALLNQIVSDFPPEHPIISSFRPLQE | |
| | | japonica | | | PLGHSPFQVFAGALVGCSIAYLMGKSV | |
| 45 | Os06g0530300 | Oryza sativa ssp. | AA | protein | AVVAVATSLGLERGFADSIFGMSVVFAAIVMYDAQGVRREVGNH | |
| | | japonica | | | ARVLNKLLTLREKITQNPDDNSLLSSTSELHSSKPETVAELVSVAEKL | |
| | | | | | GSSQGSSANPFPIHSSGTKSSRLNALQSSETEVTEFTQLKEAYTEECD | |
| | | | | | RLSESVGHTELQVAAGALLGFLVTLVVYATL | |
| 46 | Os08g0127500 | Oryza sativa ssp. | AA | protein | MGDNASASASVLAPPVGAGEGDAPSFSYLAALGNCPLVAAVLAGA | |
| | | japonica | | | IAQFIKVLTTWYKENRWDAKQLVGSGGMPSSHSATVVALAVAVGL | |
| | | | | | QEGFGSSLFATAAIFASVVMYDAFGVRLHAGKQAEVLNQIVYELPS | |
| | | | | | EHPLAETRPLRELLGHTPAQVFAGGVLGFAVATFTGMIAGLGNTGS | |
| | | | | | LP | |
| 47 | PAB00011436 | Picea abies | AA | protein | MGIAYTPRIHVHTRQQQVLLHNDFKVGSSNTGHFFRLKFEPLSKW | |
| | | | | | PSSSQSHQNTSISLISVFKNKRRRRSSWALVHACAATTPFAPFVAVV | |
| | | | | | TRVLQSILVGGEKGYYILRTTANKWVARLQEGYDMLRKAELANDV | |
| | | | | | QEGYDMLRKAELANDGIGVLQQWGGGGGEEGIRIGMALLSTTTS | |
| | | | | | MAKERISPVLGRLGANPTFMSGLLAWVIAQMLKVVTTFVVERRW | |
| | | | | | DLRMLVGSGGMPSSHAALCVALTTSVALCHGVSDSLFPVCLGFSLI | |
| | | | | | VMYDAAGVRRHAGMQAEVLNLIVEDLFKGHPVSEKKLKELLGHTP | |
| | | | | | LQVFAGACLGVLVGYLCSHSCLVAL | |
| 48 | PAB00041664 | Picea abies | AA | protein | MLSSPCNLLTTPRCSTHHVVQHQWLGHPIRLRHSKHYYSTKHSSAC | |
| | | | | | LGGPRAGLEQIAHNQVLVSATAACLIGQLSKPLASALLGKGFKWRL | |
| | | | | | AVKSGGMPSTHSASIVASATAIGLERGFSDSLFGLSVVVAGIVMYD | |
| | | | | | AQ | |
| 49 | PAB00041665 | Picea abies | AA | protein | MLLLLGSGIGDSNPIRAVPMLSSPCNLLTTPRCSTHHVVQHQWLG | |
| | | | | | HPIRLRHSKHYYSTFF | |
| 50 | PAB00057841 | Picea abies | AA | protein | MSMYGKNVDSTQIGVKADNVALLTNVGRGCHLRYKEKRWDAKRL | |
| | | | | | LGSGGMPSSHSASVTALAAAIGFHDGPGGSSFAISLVLACVVSLTIF | |
| | | | | | SL | |
| 51 | PAB00059673 | Picea abies | AA | protein | MQGVRRAVGKQAEVINMMIVSNTVPVCTDNNINSLVNDGEQILD | |
| | | | | | SMQRELNSVEDMDPSVAISQIAVARAETIRSATCFPRETEVSSVRP | |
| | | | | | HEIRISNEDGQFIDGAGLSSLSELTQTSPSVKTGKVDFQRLKQLAAW | |
| | | | | | RHIPLKESVGHTKVEVLVGGLVGLIVTLGLQWIR | |
| 52 | Pp3c14_24670 | Physcomitrium patens | AA | protein | MACLALSRTAGPSTLYLPIGSPLHPRHLFRHFVPALDNANLHPRCAL | |
| | | | | | SRQGWSIICGTSWNHHARKETSVAGQQCCQSHSCSSFSEGRSNED | |
| | | | | | VSCSAGEDADKSPTHSPLHLTTFLAQALAIVSYWRARLHRCRAELET | |
| | | | | | LCEESERETSEGALLMQGSMGMALLSISMIARDRISPVLITLRTNPT | |
| | | | | | FMSGLVAWAIAQVLKVFTKYFVERRWDWKMLVGSGGMPSSHSA | |
| | | | | | LCVGLTTAVALCHGVGDSLFPVCLGFTLIVMYDAAGVRRHAGRQA | |
| | | | | | EVLNMIVEDLFQGHPVSEKKLKELLGHTPLQVGAGAILGMICGYICS | |
| | | | | | RSSMVY | |
| 53 | Pp3c17_24060 | Physcomitrium patens | AA | protein | MACLALSRITGPSMFSPPIASPPHSRSLVQHFDPTLNTATLHSRCFV | |
| | | | | | PLQALSNTRQSSLKHRVLREKSVFGQQCCQSHGCSFGERDLNDDK | |
| | | | | | QDFTGDDAILLRHLFFHPMTLLAQALSIISYWRARLHLCRLKFEALCE | |
| | | | | | ESQWEDLDGALLQQGSLGMALLSTSMIARDRISPVLITLRANPTFM | |
| | | | | | SGLVAWAFAQVLKVFTKYFVERRWDWKMLVGSGGMPSSHSALC | |
| | | | | | VGLTTAVALCHGVGDSLFPVCLGFTLIVMYDAAGVRRHAGRQAEV | |
| | | | | | LNMIVEDLFQGHPVSEKKLKELLGHTPLQVGAGATLGMICGYICSR | |
| | | | | | ASMVY | |
| 54 | Pp3c26_3480 | Physcomitrium patens | AA | protein | MAARLLKHPAAPGACAHRNVGRRGEHDVAGQGIHRVGKLKLGG | |
| | | | | | REWLGEVNVANARLQIKQRRRGDLRMQFRAGLEELASNQVLISAA | |
| | | | | | TASTLGQLAKPFAAALAGKGFNWKLVIKSGGMPSSHAASVTAAAT | |
| | | | | | ALAFERGLSDGVFGLSVIIAGIVMYDAQGVRNAVGKQAKVINTMV | |
| | | | | | VSYVPLPQSQEEESLSAATPSSAAIGAALARDPIEAELAFMDNTYTP | |
| | | | | | SSSATQTQSSLATLEAGSPNASKSPVGAAADMNNGAYTRSSRFFEL | |
| | | | | | AGQLPSMRVGEVDIQELGNQDGWRLLPLKESIGHTKIEVLVGGIW | |
| | | | | | GIVITCIFHSLYNS | |
| 55 | Pp3c4_23210 | Physcomitrium patens | AA | protein | MLKLGRDDWLGDGNFTHTRLLLKQKRRSCRIHVRAGIEELALDQVL | |
| | | | | | VSAVTASTLAQLVKPFAAGLIGKGFNWKLIYKSGGMPSSHSAAVTA | |
| | | | | | AATALAYERGLSDGVFGLSVIVACIVMYDAQGVRNAVGKQAKVIN | |
| | | | | | TMVVSYIPQPQPQPQPQVEQSLPVEIGAELTQDPAEMVRAVLDK | |
| | | | | | NSSSKAMLNAPSLVILDGKSPSASKSYSETAADFYNGAYTRSRGFFE | |
| | | | | | LARQMPAMQVGEVDIQELGKQDGWRLLPLKESIGHTKIEVLGGGL | |
| | | | | | WGVVITCILHSLHQS | |
| 56 | Pp3c5_28440 | Physcomitrium patens | AA | protein | MRMERLLAGDMAMASGDVAANLVNAQYDGFTNFPIVAAFLSFF | |
| | | | | | VAQSLKVLTTWYKENRWDVKRLYGSGGMPSSHSATVTGLACAIGL | |
| | | | | | REGLGGPLFAIAFVLACIVMYDASGVRLQAGRQAEVLNQIVFELPP | |
| | | | | | EHPLSDSRPLKEFLGHTPPQVAAGAMLGCLIAYTLHLLSLIQAGGDK | |
| 57 | Pp3s30_800 | Physcomitrium patens | AA | protein | MVSQIGIPLHPYENFTDGDLALLLQYTKEAYVERNGFSVADILENFP | |
| | | | | | LWAALLAIGIAQFIKIPLNYFATKTWQWSLMLSTGGMPSSHSSAVT | |
| | | | | | ALSTAVGLREGFSSNMFAISAILGVIVMFDAAGVRRHAGMQAVVL | |
| | | | | | NKLVDEFNHLLEGMKSLKVRPNQEKAKKLKELLGHQPIEVLIGGWL | |
| | | | | | GIMIVVGLDDHSDWPLEWQHLPKVGPDLTIDMDRVAALKPDLVV | |
| | | | | | ASLSVPGMEANVEALQSRGIPHIVLNPSRINEIAADIRLVGEATDM | |
| | | | | | HKQADQLATLFDERVETIRHRASQYSHRPKLYWEWWPNPIYTPGE | |
| | | | | | ENWLTDVSEIAGAVNVFRDYPVANVKATREMVAERDPDHICVVW | |
| | | | | | CGIELKRIKPAMITERPEWQEMTAIRKHQVHLLEEGLYCRPSPRILD | |
| | | | | | GLDKLAVEKGFCQLAITSSDDIDVH | |
| 58 | Pp3s50_10 | Physcomitrium patens | AA | protein | VMYDALGVSLQAAPLHMLNQIVFEIPPEHPLLDSRPLKEVFGYTPP | |
| 59 | SEGI_22154 | Sequoiadendron | AA | protein | MGAFRSLRLDYWHSSLHNGFKIGGTDFLKVKHFKKKSVPSWSSRH | |
| | | giganteum | | | HYHCTRIYVVKSNNDNGNRGNNWAFIHGVPPVAPMLAALALVLQ | |
| | | | | | NMRGRVMFSGEQGSYILQRTVKKWMARLQGCRNMVKEAELGN | |
| | | | | | GRFLQQGGIGMALMSTTLMAKQKISPVLETLWANPTFMSGLLAW | |
| | | | | | AIAQMLKVVTTFVVERRWDLKMFVSSGGMPSSHSALCCALTTSVA | |
| | | | | | LCHGVSDALFPVCLGFSLIVMYDAIGVRRHAGMQAEVLNLIVEDLF | |
| | | | | | KGHPISDRKLKELLGHTPLQVIAGACLGVFVGYRCSRNCSVVF | |
| 60 | SMO142G0076 | Selaginella | AA | protein | MAIAAPLHCNWLGSRLSYRRMWISSSQRNLGAGRTVTAGLLEEIP | |
| | | moellendorffii | | | QNHVLASAALAGLSAQLVKPLTAAVAGKGLNWKLMLRSGGTPSA | |
| | | | | | HAASMVAAATALGLERGFSDSLFGFAMVVAGIVMYDAQGVRREV | |
| | | | | | GKHAEILNTIAFAQYKVSKEPSPRSSRPELLVEAPVGATKSSNAFERG | |
| | | | | | EVDSSNNGPFSRSTKFFKTAQNLPSMKEGEVSIQELGSEDGWQYIP | |
| | | | | | LKESTGHTKSQVLAGAVFGAILSVISHAIGLS | |
| 61 | SMO367G0565 | Selaginella | AA | protein | MEGAVAAADQSLDPAAVPSAYSSFSNLPLVAAFVSFVAAQSLKIVT | |
| | | moellendorffii | | | TWYKEKRWDLKRMAGSGGMPSSHSATVIGLTVAIGLRDGTGGSL | |
| | | | | | FAIALVLASIVMYDASSVRFHAGRQAEVLNQIVFELPPEHPLADSRP | |
| | | | | | LREPLGHTPPQVAAGAALGCIIAYILYLISLLGV | |
| 62 | SMO367G0696 | Selaginella | AA | protein | MASAHLSPVIVTLRANPTFMSGLVAWMVAQASKVLTTYVVYRRW | |
| | | moellendorffii | | | DLRMLVGSGGMPSSHSALCLGLTTSVALSHGVGDALFPVCLGFSLI | |
| | | | | | VMYDATGVRRHAGMQAEVLNMIIKDLFQGHPVSEKKLKELLGHT | |
| | | | | | PLQVVAGALVGILVGWWCCLTPR | |
| 63 | Solyc01g005910.3 | Solanum lycopersicum | AA | protein | MLSLQNWGYPSTVSLQNPSIYKNTTTKILVFPCLGKNGRCFSYSSLT | |
| | | | | | SKPLPKLLCLHGFGVEDITDVVHNKVLVAAAVSAAVGQLMKPFTSS | |
| | | | | | LFYGNEFDFKTAFQAGGFPSTHSSAVVATATALGLERGFSDSIFGLA | |
| | | | | | VVYAGLVMYDAQGVRREVGIHAKAFNKALFRNQINSVPSTSELDV | |
| | | | | | LTDSIQEKLSSEAENSDPQLSEESSSFQPRSKNATLLLKPDERRAPSSS | |
| | | | | | FAPLKEQVGHTEVEVIAGAFLGFFVSLAVSLA | |
| 64 | Solyc01g095980.3 | Solanum lycopersicum | AA | protein | MDSLLINSPYTALASSKILNSNITSFNLSSFCFFRFKRPNKLKTSSVTVS | |
| | | | | | YLNPQNPNSEWQNHSSYLKPFSLLLPIFKKVKNFAENNRWGSVFK | |
| | | | | | GCSGTENVPEELRGDLLQNGSFGMALLSITATAKVKISPIVATLAAN | |
| | | | | | PTFVSGFIAWFMAQSMKVFLNFCVERKWDFRIMFASGGMPSSHS | |
| | | | | | ALCTALTTSVAICHGVADSLFPVCLGFTLIVMYDAIGVRRHAGMQA | |
| | | | | | EVLNLIVEDLFQGHPISQRKLKELLGHTPLQVFAGALLGIIVAWMCS | |
| | | | | | QGYLIAI | |
| 65 | Solyc04g024340.4 | Solanum lycopersicum | AA | protein | MMTATMTTTVSVGSSSFFTNYPLMSALIAFALAQSIKLFTSWYKER | |
| | | | | | RWDLKQLVGSGGMPSSHSSTVTALAVAVGLQEGFGGALFACALVL | |
| | | | | | ACVVMYDATGVRLHAGRQAEVLNQILYELPSEHPLADSRPLRELLG | |
| | | | | | HTPPQVVAGGLLGLTTATAIHFIRGSGHEA | |
| 66 | Solyc05g014700.3 | Solanum lycopersicum | AA | protein | MMDSTVTLIQNLDPMNISTTTTTIASYGSSSFLSNCPLLSAIIAFALA | |
| | | | | | QSIKFFTSWYREKHWDLKQLVGSGGMPSSHSSTVTALATAVGLQE | |
| | | | | | GFGGSLFAISLVLACVVMYDATGVRLHAGRQAEVLNQIVCELPEEH | |
| | | | | | PLADTLPLRELLGHTPPQVIAGGFLGLVTATIVWLITSSAYRA | |
| 67 | Solyc10g006140.3 | Solanum lycopersicum | AA | protein | MNEVLTASDASSSARSYSSSIAPVNVPLFSALLACAIAQFLKLFTTWY | |
| | | | | | KEKRWDSKRMLSSGGMPSSHSATVTSLIMAIYLQEGAGGSVFAIA | |
| | | | | | VVLACVVMYDATGVRLHAGRQAELLNQIVCELPPEHPVANVRPLR | |
| | | | | | DSLGHTPLQVLAGAVLGCVVPLLLRSSI | |
| 68 | pROK2 vector | synthetic | DNA | other DNA | CCGGGCTGGTTGCCCTCGCCGCTGGGCTGGCGGCCGTCTATGG | |
| | | | | | CCCTGCAAACGCGCCAGAAACGCCGTCGAAGCCGTGTGCGAGA | |
| | | | | | CACCGC GGCCGCCGGCGTTGTGGATACCTCGCGG | |
| | | | | | AAAACTTGGCCCTCACTGACAGATGAGGGGCGGACGTTGACAC | |
| | | | | | TTGAGGGGCCGACTCACCCGGCGCGGCGTTGACAGATGAGGG | |
| | | | | | GCAGGCTCGATTTCGGCCGGCGACGTGGAGCTGGCCAGCCTCG | |
| | | | | | CAAATCGGCGAAAACGCCTGATTTTACGCGAGTTTCCCACAGAT | |
| | | | | | GATGTGGACAAGCCTGGGGATAAGTGCCCTGCGGTATTGACAC | |
| | | | | | TTGAGGGGCGCGACTACTGACAGATGAGGGGCGCGATCCTTGA | |
| | | | | | CACTTGAGGGGCAGAGTGCTGACAGATGAGGGGCGCACCTATT | |
| | | | | | GACATTTGAGGGGCTGTCCACAGGCAGAAAATCCAGCATTTGCA | |
| | | | | | AGGGTTTCCGCCCGTTTTTCGGCCACCGCTAACCTGTCTTTTAAC | |
| | | | | | CTGCTTTTAAACCAATATTTATAAACCTTGTTTTTAACCAGGGCT | |
| | | | | | GCGCCCTGTGCGCGTGACCGCGCACGCCGAAGGGGGGTGCCCC | |
| | | | | | CCCTTCTCGAACCCTCCCGGCCCGCTAACGCGGGCCTCCCATCCC | |
| | | | | | CCCAGGGGCTGCGCCCCTCGGCCGCGAACGGCCTCACCCCAAA | |
| | | | | | AATGGCAGCGCTGGCAGTCCTTGCCATTGCCGGGATCGGGGCA | |
| | | | | | GTAACGGGATGGGCGATCAGCCCGAGCGCGACGCCCGGAAGC | |
| | | | | | ATTGACGTGCCGCAGGTGCTGGCATCGACATTCAGCGACCAGG | |
| | | | | | TGCCGGGCAGTGAGGGCGGCGGCCTGGGTGGCGGCCTGCCCTT | |
| | | | | | CACTTCGGCCGTCGGGGCATTCACGGACTTCATGGCGGGGCCG | |
| | | | | | GCAATTTTTACCTTGGGCATTCTTGGCATAGTGGTCGCGGGTGC | |
| | | | | | CGTGCTCGTGTTCGGGGGTGCGATAAACCCAGCGAACCATTTGA | |
| | | | | | GGTGATAGGTAAGATTATACCGAGGTATGAAAACGAGAATTGG | |
| | | | | | ACCTTTACAGAATTACTCTATGAAGCGCCATATTTAAAAAGCTAC | |
| | | | | | CAAGACGAAGAGGATGAAGAGGATGAGGAGGCAGATTGCCTT | |
| | | | | | GAATATATTGACAATACTGATAAGATAATATATCTTTTATATAGA | |
| | | | | | AGATATCGCCGTATGTAAGGATTTCAGGGGGCAAGGCATAGGC | |
| | | | | | AGCGCGCTTATCAATATATCTATAGAATGGGCAAAGCATAAAAA | |
| | | | | | CTTGCATGGACTAATGCTTGAAACCCAGGACAATAACCTTATAG | |
| | | | | | CTTGTAAATTCTATCATAATTGGGTAATGACTCCAACTTATTGAT | |
| | | | | | AGTGTTTTATGTTCAGATAATGCCCGATGACTTTGTCATGCAGCT | |
| | | | | | CCACCGATTTTGAGAACGACAGCGACTTCCGTCCCAGCCGTGCC | |
| | | | | | AGGTGCTGCCTCAGATTCAGGTTATGCCGCTCAATTCGCTGCGT | |
| | | | | | ATATCGCTTGCTGATTACGTGCAGCTTTCCCTTCAGGCGGGATTC | |
| | | | | | ATACAGCGGCCAGCCATCCGTCATCCATATCACCACGTCAAAGG | |
| | | | | | GTGACAGCAGGCTCATAAGACGCCCCAGCGTCGCCATAGTGCG | |
| | | | | | TTCACCGAATACGTGCGCAACAACCGTCTTCCGGAGACTGTCAT | |
| | | | | | A CGCGTAAAACAGCCAGCGCTGGCGCGATTTA | |
| | | | | | GCCCCGACATAGCCCCACTGTTCGTCCATTTCCGCGCAGACGAT | |
| | | | | | GACGTCACTGCCCGGCTGTATGCGCGAGGTTACCGACTGCGGC | |
| | | | | | CTGAGTTTTTTAAGTGACGTAAAATCGTGTTGAGGCCAACGCCC | |
| | | | | | ATAATGCGGGCTGTTGCCCGGCATCCAACGCCATTCATGGCCAT | |
| | | | | | ATCAATGATTTTCTGGTGCGTACCGGGTTGAGAAGCGGTGTAAG | |
| | | | | | TGAACTGCA GTTGCCATGTTT | |
| | | | | | TACGGCAGTGAGAGCAGAGATAGCGCTGATGTCCGGCGGTGCT | |
| | | | | | TTTGCCGTTACGCACCACCCCGTCAGTAGCTGAACAGGAGGGAC | |
| | | | | | AGCTGATAGACACAGAAGCCACTGGAGCACCTCAAAAACACCA | |
| | | | | | TCATACACTAAATCAGTAAGTTGGCAGCATCACCCATAATTGTG | |
| | | | | | GTTTCAAAATCGGCTCCGTCGATACTATGTTATACGCCAACTTTG | |
| | | | | | AAAACAACTTTGAAAAAGCTGTTTTCTGGTATTTAAGGTTTTAGA | |
| | | | | | ATGCAAGGAACAGTGAATTGGAGTTCGTCTTGTTATAATTAGCT | |
| | | | | | TCTTGGGGTATCTTTAAATACTGTAGAAAAGAGGAAGGAAATAA | |
| | | | | | TAAATGGCTAAAATGAGAATATCACCGGAATTGAAAAAACTGAT | |
| | | | | | CGAAAAATACCGCTGCGTAAAAGATACGGAAGGAATGTCTCCT | |
| | | | | | GCTAAGGTATATAAGCTGGTGGGAGAAAATGAAAACCTATATTT | |
| | | | | | AAAAATGACGGACAGCCGGTATAAAGGGACCACCTATGATGTG | |
| | | | | | GAACGGGAAAAGGACATGATGCTATGGCTGGAAGGAAAGCTG | |
| | | | | | CCTGTTCCAAAGGTCCTGCACTTTGAACGGCATGATGGCTGGAG | |
| | | | | | CAATCTGCTCATGAGTGAGGCCGATGGCGTCCTTTGCTCGGAAG | |
| | | | | | AGTATGAAGATGAACAAAGCCCTGAAAAGATTATCGAGCTGTAT | |
| | | | | | GCGGAGTGCATCAGGCTCTTTCACTCCATCGACATATCGGATTG | |
| | | | | | TCCCTATACGAATAGCTTAGACAGCCGCTTAGCCGAATTGGATT | |
| | | | | | ACTTACTGAATAACGATCTGGCCGATGTGGATTGCGAAAACTGG | |
| | | | | | GAAGAAGACACTCCATTTAAAGATCCGCGCGAGCTGTATGATTT | |
| | | | | | TTTAAAGACGGAAAAGCCCGAAGAGGAACTTGTCTTTTCCCACG | |
| | | | | | GCGACCTGGGAGACAGCAACATCTTTGTGAAAGATGGCAAAGT | |
| | | | | | AAGTGGCTTTATTGATCTTGGGAGAAGCGGCAGGGCGGACAAG | |
| | | | | | TGGTATGACATTGCCTTCTGCGTCCGGTCGATCAGGGAGGATAT | |
| | | | | | CGGGGAAGAACAGTATGTCGAGCTATTTTTTGACTTACTGGGGA | |
| | | | | | TCAAGCCTGATTGGGAGAAAATAAAATATTATATTTTACTGGAT | |
| | | | | | GAATTGTTTTAGTACCTAGATGTGGCGCAACGATGCCGGCGACA | |
| | | | | | AGCAGGAGCGCACCGACTTCTTCCGCATCAAGTGTTTTGGCTCT | |
| | | | | | CAGGCCGAGGCCCACGGCAAGTATTTGGGCAAGGGGTCGCTGG | |
| | | | | | TATTCGTGCAGGGCAAGATTCGGAATACCAAGTACGAGAAGGA | |
| | | | | | CGGCCAGACGGTCTACGGGACCGACTTCATTGCCGATAAGGTG | |
| | | | | | GATTATCTGGACACCAAGGCACCAGGCGGGTCAAATCAGGAAT | |
| | | | | | AAGGGCACATTGCCCCGGCGTGAGTCGGGGCAATCCCGCAAG3 | |
| | | | | | 361 | |
| | | | | | GAGGGTGAATGAATCGGACGTTTGACCGGAAGGCATACAGGCA | |
| | | | | | AGAACTGATCGACGCGGGGTTTTCCGCCGAGGATGCCGAAACC | |
| | | | | | ATCGCAAGCCGCACCGTCATGCGTGCGCCCCGCGAAACCTTCCA | |
| | | | | | GTCCGTCGGCTCGATGGTCCAGCAAGCTACGGCCAAGATCGAG | |
| | | | | | CGCGACAGCGTGCAACTGGCTCCCCCTGCCCTGCCCGCGCCATC | |
| | | | | | GGCCGCCGTGGAGCGTTCGCGTCGTCTCGAACAGGAGGCGGCA | |
| | | | | | GGTTTGGCGAAGTCGATGACCATCGACACGCGAGGAACTATGA | |
| | | | | | CGACCAAGAAGCGAAAAACCGCCGGCGAGGACCTGGCAAAAC | |
| | | | | | AGGTCAGCGAGGCCAAGCAGGCCGCGTTGCTGAAACACACGAA | |
| | | | | | GCAGCAGATCAAGGAAATGCAGCTTTCCTTGTTCGATATTGCGC | |
| | | | | | CGTGGCCGGACACGATGCGAGCGATGCCAAACGACACGGCCCG | |
| | | | | | CTCTGCCCTGTTCACCACGCGCAACAAGAAAATCCCGCGCGAGG | |
| | | | | | CGCTGCAAAACAAGGTCATTTTCCACGTCAACAAGGACGTGAAG | |
| | | | | | ATCACCTACACCGGCGTCGAGCTGCGGGCCGACGATGACGAAC | |
| | | | | | TGGTGTGGCAGCAGGTGTTGGAGTACGCGAAGCGCACCCCTAT | |
| | | | | | CGGCGAGCCGATCACCTTCACGTTCTACGAGCTTTGCCAGGACC | |
| | | | | | TGGGCTGGTCGATCAATGGCCGGTATTACACGAAGGCCGAGGA | |
| | | | | | ATGCCTGTCGCGCCTACAGGCGACGGCGATGGGCTTCACGTCC | |
| | | | | | GACCGCGTTGGGCACCTGGAATCGGTGTCGCTGCTGCACCGCTT | |
| | | | | | CCGCGTCCTGGACCGTGGCAAGAAAACGTCCCGTTGCCAGGTCC | |
| | | | | | TGATCGACGAGGAAATCGTCGTGCTGTTTGCTGGCGACCACTAC | |
| | | | | | ACGAAATTCA | |
| | | | | | TATGGGAGAAGTACCGCAAGCTGTCGCCGACGGCCCGAC | |
| | | | | | GGATGTTCGACTATTTCAGCTCGCACCGGGAGCCGTACCCGCTC | |
| | | | | | AAGCTGGAAACCTTCCGCCTCATGTGCGGATCGGATTCCACCCG | |
| | | | | | CGTGAAGAAGTGGCGCGAGCAGGTCGGCGAAGCCTGCGAAGA | |
| | | | | | GTTGCGAGGCAGCGGCCTGGTGGAACACGCCTGGGTCAATGAT | |
| | | | | | GACCTGGTGCATTGCAAACGCTAGGGCCTTGTGGGGTCAGTTCC | |
| | | | | | GGCTGGGGGTTCAGCAGCCAGCGCTTTACTGGCATTTCAGGAA | |
| | | | | | CAAGCGGGCACTGCTCGACGCACTTGCTTCGCTCAGTATCGCTC | |
| | | | | | GGGACGCACGGCGCGCTCTACGAACTGCCGATAAACAGAGGAT | |
| | | | | | TAAAATTGACAATTGTGATTAAGGCTCAGATTCGACGGCTTGGA | |
| | | | | | GCGGCCGACGTGCAGGATTTCCGCGAGATCCGATTGTCGGCCCT | |
| | | | | | GAAGAAAGCTCCAGAGATGTTCGGGTCCGTTTACGAGCACGAG | |
| | | | | | GAGAAAAAGCCCATGGAGGCGTTCGCTGAACGGTTGCGAGATG | |
| | | | | | CCGTGGCATTCGGCGCCTACATCGACGGCGAGATCATTGGGCT | |
| | | | | | GTCGGTCTTCAAACAGGAGGACGGCCCCAAGGACGCTCACAAG | |
| | | | | | GCGCATCTGTCCGGCGTTTTCGTGGAGCCCGAACAGCGAGGCC | |
| | | | | | GAGGGGTCGCCGGTATGCTGCTGCGGGCGTTGCCGGCGGGTTT | |
| | | | | | ATTGCTCGTGATGATCGTCCGACAGATTCCAACGGGAATCTGGT | |
| | | | | | GGATGCGCATCTTCATCCTCGGCGCACTTAATATTTCGCTATTCT | |
| | | | | | GGAGCTTGTTGTTTATTTCGGTCTACCGCCTGCCGGGGGGGGTC | |
| | | | | | GCGGCGACGGTAGGCGCTGTGCAGCCGCTGATGGTCGTGTTCA | |
| | | | | | TCTCTGCCGCTCTGCTAGGTAGCCCGATACGATTGATGGCGGTC | |
| | | | | | CTGGGGGCTATTTGCGGAACTGCGGGCGTGGCGCTGTTGGTGT | |
| | | | | | TGACACCAAACGCAGCGCTAGATCCTGTCGGCGTCGCAGCGGG | |
| | | | | | CCTGGCGGGGGCGGTTTCCATGGCGTTCGGAACCGTGCTGACC | |
| | | | | | CGCAAGTGGCAACCTCCCGTGCCTCTGCTCACCTTTACCGCCTGG | |
| | | | | | CAACTGGCGGCCGGAGGACTTCTGCTCGTTCCAGTAGCTTTAGT | |
| | | | | | GTTTGATCCGCCAATCCCGATGCCTACAGGAACCAATGTTCTCG | |
| | | | | | GCCTGGCGTGGCTCGGCCTGATCGGAGCGGGTTTAACCTACTTC | |
| | | | | | CTTTGGTTCCGGGGGATCTCGCGACTCGAACCTACAGTTGTTTCC | |
| | | | | | TTACTGGGCTTTCTCAGCCCCA | |
| | | | | | GATCTGGGGTCGATCAGCCGGGGATGCATCAGGC | |
| | | | | | CGACAGTCGGAACTTCGGGTCCCCGACCTGTACCATTCGGTGAG | |
| | | | | | CAATGGATAGGGGAGTTGATATCGTCAACGTTCACTTCTAAAGA | |
| | | | | | AATAGCGCCACTCAGCTTCCTCAGCGGCTTTATCCAGCGATTTCC | |
| | | | | | TATTATGTCGGCATAGTTCTCAAGATCGACAGCCTGTCACGGTT | |
| | | | | | AAGCGAGAAATGAATAAGAAGGCTGATAATTCGGATCTCTGCG | |
| | | | | | AGGGAGATGATATTTGATCACAGGCAGCAACGCTCTGTCATCGT | |
| | | | | | TACAATCAACATGCTACCCTCCGCGAGATCATCCGTGTTTCAAAC | |
| | | | | | CCGGCAGCTTAGTTGCCGTTCTTCCGAATAGCATCGGTAACATG | |
| | | | | | AGCAAAGTCTGCCGCCTTACAACGGCTCTCCCGCTGACGCCGTC | |
| | | | | | CCGGACTGATGGGCTGCCTGTAT | |
| | | | | | CGAGTGGTGATTTTGTGCCGAGCTGCCGGTCGGGGAGCTGTTG | |
| | | | | | GCTGGCTGGTGGCAGGATATATTGTGGTGTAAACAAATTGACG | |
| | | | | | CTTAGACAACTTAATAACACATTGCGGACGTTTTTAATGTACTGG | |
| | | | | | GGTGGTTTTTCTTTTCACCAGTGAGACGGGCAACAGCTGATTGC | |
| | | | | | CCTTCACCGCCTGGCCCTGAGAGAGTTGCAGCAAGCGGTCCACG | |
| | | | | | CTGGTTTGCCCCAGCAGGCGA | |
| | | | | | AAATCCTGTTTGATGGTGGTTCCGAAATCGGCAAAATCCCTTAT | |
| | | | | | AAATCAAAAGAATAGC | |
| | | | | | CCGAGATAGGGTTGAGTGTTGTTCCAGTTTGGAACAAGAGTCCA | |
| | | | | | CTATTAAAGAACGTGGACTCCAACGTCAAAGGGCGAAAAACCG | |
| | | | | | TCTATCAGGGCGATGGCCCACTACGTGAACCAT | |
| | | | | | CACCCAAATCAAGTTTTTTGGGGTCGAGGTGCCGTAAAGCACTA | |
| | | | | | AATCGGAACCCTAAAGGGAGCCCCCGATTTAGAGCTTGACGGG | |
| | | | | | GAAAGCCGGCGAACGTGGCGAGAAAGGAAGGGAAGAAAGCG | |
| | | | | | AAAGGAGCGGGCGCCATTCAGGCTGCGCAACTGTTGGGAAGG | |
| | | | | | GCGATCGGTGCGGGCCTCTTCGCTATTACGCCAGCTGGCGAAA | |
| | | | | | GGGGGATGTGCTGCAAGGCGATTAAGTTGGGTAACGCCAGGGT | |
| | | | | | TTTCCCAGTCACGACGTTGTAAAACGACGGCCAGTG | |
| | | | | | AATTCCCG | |
| | | | | | ATCTAGTAACATAGATGACACCGCGCGCGATAATTTATCCTAGTT | |
| | | | | | TGCGCGCTATATTTTGTTTTCTATCGCGTATTAAATGTATAATTGC | |
| | | | | | GGGACTCTAATCATAAAAACCCATCTCATAAATAACGTCATGCAT | |
| | | | | | TACATGTTAATTATTACATGCTTAACGTAATTCAACAGAAATTAT | |
| | | | | | ATGATAATCATCGCAAGACCGGCAACAGGATTCAATCTTAAGAA | |
| | | | | | ACTTTATTGCCAAATGTTTGAACGATCGGGGAAATTCGAGCTCG | |
| | | | | | GTACCCGGG GATCCTCTAGAGTCCCCCGTGT | |
| | | | | | TCTCTCCAAATGAAATGAACTTCCTTATATAGAGGAAGGGTCTT | |
| | | | | | GCGAAGGATAGTGGGATTGTGCGTCATCCCTTACGTCAGTGGA | |
| | | | | | GATATCACATCAATCCACTTGCTTTGAAGACGTGGTTGGAACGT | |
| | | | | | CTTCTTTTTCCACGATGCTCCTCGTGGGTGGGGGTCCATCTTTGG | |
| | | | | | GACCACTGTCGGCAGAGGCATCTTCAACGATGGCCTTTCCTTTAT | |
| | | | | | CGCAATGATGGCATTTGTAGGAGCCACCTTCCTTTTCCACTATCT | |
| | | | | | TCACAATAAAGTGACAGATAGCTGGGCAATGGAATCCGAGGAG | |
| | | | | | GTTTCCGGATATTACCCTTTGTTGAAAAGTCTCAATTGCCCTTTG | |
| | | | | | GTCTTCTGAGACTGTATCTTTGATATTTTTGGAGTAGACAAGTGT | |
| | | | | | GTCGTGCTCCACCATGTTGACGAAGATTTTCTTCTTGTCATTGAG | |
| | | | | | TCGTAAGAGACTCTGTATGAACTGTTCGCCAGTCTTTACGGCGA | |
| | | | | | GTTCTGTTAGGTCCTCTATTTGAATCTTTGACTCCATGGCCTTTGA | |
| | | | | | TTCAGTGGGAACTACCTTTTTAGAGACTCCAATCTCTATTACTTG | |
| | | | | | CCTTGGTTTGTGAAGCAAGCCTTGAATCGTCCATACTGGAATAG | |
| | | | | | TACTTCTGATCTTGAGAAATATATCTTTCTCTGTGTTCTTGATGCA | |
| | | | | | GTTAGTCCTGAATCTTTTGACTGCATCTTTAACCTTCTTGGGAAG | |
| | | | | | GTATTTGATTTCCTGGAGATTATTGCTCGGGTAGATCGTCTTGAT | |
| | | | | | GAGACCTGCTGCGTAAGCCTCTCTAACCATCTGTGGGTTAGCAT | |
| | | | | | TCTTTCTGAAATTGAAAAGGCTAATCTGGGGACCTGCAGGCATG | |
| | | | | | CA | |
| | | | | | AGCTTGGCGTAATCATGGTCATAGCTGTTTCCTGTGTGAAATTGT | |
| | | | | | TATCCG | |
| | | | | | CTCACAATTCCACACAACATACGAGCCGGAAGCATAAAGTGTAA | |
| | | | | | AGCCTGGGGTGCCTAATGAGTGAGCTAACTCACATTAATTGCGT | |
| | | | | | TGCGCTCACTGCCCGCTTTCCAGTCGGGAAACCTGTCGTGCCAG | |
| | | | | | CTGCATTAATGAATCGGCCAACGCGCGGGGAGAGGCGGTTTGC | |
| | | | | | GTATTGGGCCAAAGACAAAAGGGCGACATTCAACCGATTGAGG | |
| | | | | | GAGGGAAGGTAAATATTGACGGAAATTATTCATTAAAGGTGAA | |
| | | | | | TTATCACCGTCACCGACTTGAGCCATTTGGGAATTAGAG8281 | |
| | | | | | CCAGCAAAATCACCAGTAGCACCATTACCATTAGCAAGGCCGGA | |
| | | | | | AACGTCACCAATGAAACCATCGATAGCAGCACCGTAATCAGTAG | |
| | | | | | CGACAGAATCAAGTTTGCCTTTAGCGTCAGACTGTAGCGCGTTT | |
| | | | | | TCATCGGCATTTTCGGTCATAGCCCCCTTATTAGCGTTTGCCATC | |
| | | | | | TTTTCATAATCAAAATCACCGGAACCAGAGCCACCACCGGAACC | |
| | | | | | GCCTCCCTCAGAGCCGCCACCCTCAGAACCGCCACCCTCAGAGC | |
| | | | | | CACCACCCTCAGAGCCGCCACCAGAACCACCACCAGAGCCGCCG | |
| | | | | | CCAGCATTGACAGGAGGCCCGATCTAGTAACATAGATGACACC | |
| | | | | | GCGCGCGATAATTTATCCTAGTTTGCGCGCTATATTTTGTTTTCT | |
| | | | | | ATCGCGTATTAAATGTATAATTGCGGGACTCTAATCATAAAAAC | |
| | | | | | CCATCTCATAAATAACGTCATGCATTACATGTTAATTATTACATG | |
| | | | | | CTTAACGTAATTCAACAGAAATTATATGATAATCATCGCAAGACC | |
| | | | | | GGCAACAGGATTCAATCTTAAGAAACTTTATTGCCAAATGTTTG | |
| | | | | | AACGATCGGGGATCATCCGGGTCTGTGGCGGGAACTCCACGAA | |
| | | | | | AATATCCGAACGCAGCAAGATATCGCGGTGCATCTCGGTCTTGC | |
| | | | | | CTGGGCAGTCGCCGCCGACGCCGTTGATGTGGACGCCGGGCCC | |
| | | | | | GATCATATTGTCGCTCAGGATCGTGGCGTTGTGCTTGTCGGCCG | |
| | | | | | TTGCTGTCGTAATGATATCGGCACCTTCGACCGCCTGTTCCGCAG | |
| | | | | | AGATCCCGTGGGCGAAGAACTCCAGCATGAGATCCCCGCGCTG | |
| | | | | | GAGGATCATCCAGCCGGCGTCCCGGAAAACGATTCCGAAGCCC | |
| | | | | | AACCTTTCATAGAAGGCGGCGGTGGAATCGAAATCTCGTGATG | |
| | | | | | GCAGGTTGGGCGTCGCTTGGTCGGTCATTTCGAACCCCAGAGTC | |
| | | | | | CCGCTCAGAAGAACTCGTCAAGAAGGCGATAGAAGGCGATGCG | |
| | | | | | CTGCGAATCG | |
| | | | | | GGAGCGGCGATACCGTAAAGCACGAGGAAGCGGTCAGCCCATT | |
| | | | | | CGCCGCCAAGCTCTTCAGCAATATCACGGGTAGCCAACGCTATG | |
| | | | | | TCCTGATAGCGGTCCGCCACACCCAGCCGGCCACAGTCGATGAA | |
| | | | | | TCCAGAAAAGCGGCCATTTTCCACCATGATATTCGGCAAGCAGG | |
| | | | | | CATCGCCATGGGTCACGACGAGATCATCGCCGTCGGGCATGCG | |
| | | | | | CGCCTTGAGCCTGGCGAACAGTTCGGCTGGCGCGAGCCCCTGA | |
| | | | | | TGCTCTTCGTCCAGATCATCCTGATCGACAAGACCGGCTTCCATC | |
| | | | | | CGAGTACGTGCTCGCTCGATGCGATGTTTCGCTTGGTGGTCGAA | |
| | | | | | TGGGCAGGTAGCCGGATCAAGCGTATGCAGCCGCCGCATTGCA | |
| | | | | | TCAGCCATGATGGATACTTTCTCGGCAGGAGCAAGGTGAGATG | |
| | | | | | ACAGGAGATCCTGCCCCGGCACTTCGCCCAATAGCAGCCAGTCC | |
| | | | | | CTTCCCGCTTCAGTGACAACGTCGAGCACAGCTGCGCAAGGAAC | |
| | | | | | GCCCGTCGTGGCCAGCCACGATAGCCGCGCTGCCTCGTCCTGCA | |
| | | | | | GTTCATTCAGGGCACCGGACAGGTCGGTCTTGACAAAAAGAAC | |
| | | | | | CGGGCGCCCCTGCGCTGACAGCCGGAACACGGCGGCATCAGAG | |
| | | | | | CAGCCGATTGTCTGTTGTGCCCAGTCATAGCCGAATAGCCTCTCC | |
| | | | | | ACCCAAGCGGCCGGAGAACCTGCGTGCAATCCATCTTGTTCAAT | |
| | | | | | CATGCGAAACGATCCAGATCCGGTGCAGATTATTTGG | |
| | | | | | ATTGAGAGTGAATATGAGACTCTAATTGGATACCGAGGGGAAT | |
| | | | | | TTATGGAACGTCAGTGGAGCATTTTTGACAAGAAATATTTGCTA | |
| | | | | | GCTGATAGTGACCTTAGGCGACTTTTGAACGCGCAATAATGGTT | |
| | | | | | TCTGACGTATGTGCTTAGCTCATTAAACTCCAGAAACCCGCGGC | |
| | | | | | TGAGTGGCTCCTTCAACGTTGCGGTTCTGTCAGTTCCAAACGTA | |
| | | | | | AAACGGCTTGTCCCGCGTCATCGGCGGGGGTCATAACGTGACTC | |
| | | | | | CCTTAATTCTCCGCTCATGATCAGATTGTCGTTTCCCGCCTTCAGT | |
| | | | | | TTAAACTATCAGTGTTTGACAGGATATATTGGCGGGTAAACCTA | |
| | | | | | AGAGAAAAGAGCGTTTATTAGAATAATCGGATATTTAAAAGGG | |
| | | | | | CGTGAAAAGGTTTATCCGTTCGTCCATTTGTATGTGCATGCCAAC | |
| | | | | | CACAGGGTTCCCCA GATCTGGCGCCGGCCAGCGAGACGA | |
| | | | | | GCAAGATTGGCCGCCGCCCGAAACGATCCGACAGCGCGCCCAG | |
| | | | | | CACAGGTGCGCAGGCAAATTGCACCAACGCATACAGCGCCAGC | |
| | | | | | AGAATGCCATAGTGGGCGGTGACGTCGTTCGAGTGAACCAGAT | |
| | | | | | CGCGCAGGAGGCCCGGCAGCACCGGCATAATCAGGCCGATGCC | |
| | | | | | GACAGCGTCGAGCGCGACAGTGCTCAGAATTACGATCAGGGGT | |
| | | | | | ATGTTGGGTTTCACGTCTGGCCTCCGGACCAGCCTCCGCTGGTC | |
| | | | | | CGATTGAACGCGCGGATTCTTTATCACTGATAAGTTGGTGGACA | |
| | | | | | TATTATGTTTATCAGTGATAAAGTGTCAAGCATGACAAAGTTGC | |
| | | | | | AGCCGAATACAGTGATCCGTGCCGCCCTGGACCTGTTGAACGA | |
| | | | | | GGTCGGCGTAGACGGTCTGACGACACGCAAACTGGCGGAACGG | |
| | | | | | TTGGGGGTTCAGCAGCCGGCGCTTTACTGGCACTTCAGGAACAA | |
| | | | | | GCGGGCGCTGCTCGACGCACTGGCCGAAGCCATGCTGGCGGAG | |
| | | | | | AATCATACGCATTCGGTGCCGAGAGCCGACGACGACTGGCGCT | |
| | | | | | CATTTCTGATCGGGAATGCCCGCAGCTTCAGGCAGGCGCTGCTC | |
| | | | | | GCCTACCGCGATGGCGCGCGCATCCATGCCGGCACGCGACCGG | |
| | | | | | GCGCACCGCAGATGGAAACGGCCGACGCGCAGCTTCGCTTCCT | |
| | | | | | CTGCGAGGCGGGTTTTTCGGCCGGGGACGCCGTCAATGCGCTG | |
| | | | | | ATGACAATCAGCTACTTCACTGTTGGGGCCGTGCTTGAGGAGCA | |
| | | | | | GGCCGGCGACAGCGATGCCGGCGAGCGCGGCGGCACCGTTGA | |
| | | | | | ACAGGCTCCGCTCTCGCCGCTGTTGCGGGCCGCGATAGACGCCT | |
| | | | | | TCGACGAAGCCGGTCCGGACGCAGCGTTCGAGCAGGGACTCGC | |
| | | | | | GGTGATTGTCGATGGATTGGCGAAAAGGAGGCTCGTTGTCAGG | |
| | | | | | AACGTTGAAGGACCGAGAAAGGGTGACGATTGATCAGGACCGC | |
| | | | | | TGCCGGAGCGCAACCCACTCACTACAGCAGAGCCATGTAGACA | |
| | | | | | ACATCCCCTCCCCCTTTCCACCGCGTCAGACGCCCGTAGCAGCCC | |
| | | | | | GCTACGGGCTTTTTCATGCCCTGCCCTAGCGTCCAAGCCTCACG | |
| | | | | | GCCGCGCTCGGCCTCTCTGGCGGCCTTCTGGCGCTCTTCCGCTTC | |
| | | | | | CTCGCTCACTGACTCGCTGCGCTCGGTCGTTCGGCTGCGGCGAG | |
| | | | | | CGGTATCAGCTCACTCAAAGGCGGTAATACGGTTATCCACAGAA | |
| | | | | | TCAGGGGATAACGCAGGAAAGAACATGTGAGCAAAAGGCCAG | |
| | | | | | CAAAAGGCCAGGAACCGTAAAAAGGCCGCGTTGCTGGCGTTTT | |
| | | | | | TCCATAGGCTCCGCCCCCCTGACGAGCATCACAAAAATCGACGC | |
| | | | | | TCAAGTCAGAGGTGGCGAAACCCGACAGGACTATAAAGATACC | |
| | | | | | AGGCGTTTCCCCCTGGAAGCTCCCTCGTGCGCTCTCCTGTTCCGA | |
| | | | | | CCCTGCCGCTTACCGGATACCTGTCCGCCTTTCTCCCTTCGGGAA | |
| | | | | | GCGTGGCGCTTTTCCGCTGCATAACCCTGCTTCGGGGTCATTATA | |
| | | | | | GCGATTTTTTCGGTATATCCATCCTTTTTCGCACGATATACAGGA | |
| | | | | | TTTTGCCAAAGGGTTCGTGTAGACTTTCCTTGGTGTATCCAACGG | |
| | | | | | CGTCAGCCGGGCAGGATAGGTGAAGTAGGCCCACCCGCGAGCG | |
| | | | | | GGTGTTCCTTCTTCACTGTCCCTTATTCGCACCTGGCGGTGCTCA | |
| | | | | | ACGGGAATCCTGCTCTGCGAGGCTGGCCGGCTACCGCCGGCGT | |
| | | | | | AACAGATGAGGGCAAGCGGATGGCTGATGAAACCAAGCCAACC | |
| | | | | | AGGAAGGGCAGCCCACCTATCAAGGTGTACTGCCTTCCAGACG | |
| | | | | | AACGAAGAGCGATTGAGGAAAAGGCGGCGGCGGCCGGCATGA | |
| | | | | | GCCTGTCGGCCTACCTGCTGGCCGTCGGCCAGGGCTACAAAATC | |
| | | | | | ACGGGCGTCGTGGACTATGAGCACGTCCGCGAGCTGGCCCGCA | |
| | | | | | TCAATGGCGACCTGGGCCGCCTGGGCGGCCTGCTGAAACTCTG | |
| | | | | | GCTCACCGACGACCCGCGCACGGCGCGGTTCGGTGATGCCACG | |
| | | | | | ATCCTCGCCCTGCTGGCGAAGATCGAAGAGAAGCAGGACGAGC | |
| | | | | | TTGGCAAGGTCATGATGGGCGTGGTCCGCCCGAGGGCAGAGCC | |
| | | | | | ATGACTTTTTTAGCCGCTAAAACGGCCGGGGGGTGCGCGTGATT | |
| | | | | | GCCAAGCACGTCCCCATGCGCTCCATCAAGAAGAGCGACTTCGC | |
| | | | | | GGAGCTGGTGAAGTACATCACCGACGAGCAAGGCAAGACCGA | |
| | | | | | GCGCCTTTGCGACGCTCA | |
| 69 | AtSEA1 | Arabidopsis thaliana | AA | Protein | NPTFVSAVVAWFFAQSSKMVINFFIERKWDFRLLYASGGMPSSHS | |
| | (AT3G61770) | (Columbia-0 ecotype) | | | ALCMALTTSVALCHGVADSLFPVCLGFSLIVMYDAIGVRRHAGMQ | |
| | DUF212 | | | | AEVLNLIIRDLFEGHPISQRKLKELLGHTPSQVLAGALVGIVI | |
| | AtSEA2a | | | | | |
| 70 | (AT1G24350) | Arabidopsis thaliana | AA | Protein | FTNYPLISAVTSFTIAQFIKLFTSWYRERRWDLKQLIGSGGMPSSHS | |
| | DUF212 | (Columbia-0 ecotype) | | | ATVTALAVAIGLQEGFGGSHFAIALILASVVMYDATGVRLHAGRQA | |
| | AtSEA2b | | | | EVLNQIVYELPAEHPLAESRPLRELLGHTPPQVVAGGMLGSAT | |
| 71 | (AT1G67600) | Arabidopsis thaliana | AA | Protein | FTNYPLISAVLAFTIAQFIKFFTSWYKERRWDLKRLVGSGGMPSSHS | |
| | DUF212 | (Columbia-0 ecotype) | | | ATVTALALAVGLQEGFGGSHFAIALVLTTIVMYDATGVRLHAGRQA | |
| | | | | | EVLNQIVYELPAEHPLAETRPLRELLGHTPPQVIAGGMLGIST | |
| 72 | AtSEA2c | Arabidopsis thaliana | AA | Protein | HNLPIFSAFLAFALAQFLKVFTNWYKEKRWDSKRMISSGGMPSSH | |
| | (AT3G21610) | (Columbia-0 ecotype) | | | SATVTALAVAIGFEEGAGAPAFAIAVVLACVVMYDASGVRLHAGR | |
| | DUF212 | | | | QAELLNQIVCEFPPEHPLSTVRPLRELLGHTPIQVAAGGILGCVV | |
| 73 | AtSEA3 | Arabidopsis thaliana | AA | Protein | IHNKVLIAAGTSAVIGQLSKPFTSVVLYGKNLDFRSVFQAGGFPSTH | |
| | (AT3G12685) | (Columbia-0 ecotype) | | | SSSVVAAATAIAFERGFADSIFGLTVVYAGLIMYDAQGVRREVGKH | |
| | DUF212 | | | | AKVLNKLTANARRSEVMSLKGNESNKALTSEEISEEIAPPLKESIGHT | |
| | | | | | EVEVIAGALFGFLV | |
| 74 | Solyc01g095980.3 | Solanum lycopersicum | AA | Protein | LLSITATAKVKISPIVATLAANPTFVSGFIAWFMAQSMKVFLNFCVE | |
| | DUF212 | | | | RKWDFRIMFASGGMPSSHSALCTALTTSVAICHGVADSLFPVCLGF | |
| | | | | | TLIVMYDAIGVRRHAGMQAEVLNLIVEDLFQGHPISQRKLKELLGH | |
| | | | | | TPLQVFAGALLGIIVAWMCSQ | |
| 75 | Solyc04g024340.4 | Solanum lycopersicum | AA | Protein | TMTTTVSVGSSSFFTNYPLMSALIAFALAQSIKLFTSWYKERRWDLK | |
| | DUF212 | | | | QLVGSGGMPSSHSSTVTALAVAVGLQEGFGGALFACALVLACVV | |
| | | | | | MYDATGVRLHAGRQAEVLNQILYELPSEHPLADSRPLRELLGHTPP | |
| | | | | | QVVAGGLLGLTTATAIHF | |
| 76 | Solyc05g014700.3 | Solanum lycopersicum | AA | Protein | TTTTTIASYGSSSFLSNCPLLSAIIAFALAQSIKFFTSWYREKHWDLKQ | |
| | DUF212 | | | | LVGSGGMPSSHSSTVTALATAVGLQEGFGGSLFAISLVLACVVMYD | |
| | | | | | ATGVRLHAGRQAEVLNQIVCELPEEHPLADTLPLRELLGHTPPQVIA | |
| | | | | | GGFLGLVTATIV | |
| 77 | Solyc10g006140.3 | Solanum lycopersicum | AA | Protein | ASSSARSYSSSIAPVNVPLFSALLACAIAQFLKLFTTWYKEKRWDSKR | |
| | DUF212 | | | | MLSSGGMPSSHSATVTSLIMAIYLQEGAGGSVFAIAVVLACVVMY | |
| | | | | | DATGVRLHAGRQAELLNQIVCELPPEHPVANVRPLRDSLGHTPLQ | |
| | | | | | VLAGAVLGCVVPLLLRS | |
| 78 | Solyc01g005910.3 | Solanum lycopersicum | AA | Protein | VEDITDVVHNKVLVAAAVSAAVGQLMKPFTSSLFYGNEFDFKTAF | |
| | DUF212 | | | | QAGGFPSTHSSAVVATATALGLERGFSDSIFGLAVVYAGLVMYDA | |
| | | | | | QGVRREVGIHAKAFNKALFRNQINSVPSTSELDVLTDSIQEKLSSEA | |
| | | | | | ENSDPQLSEESSSFQPRSKNATLLLKPDERRAPSSSFAPLKEQVGHT | |
| | | | | | EVEVIAGAFLGFFVSLAVS | |
| 79 | Os05g0534100 | Oryza sativa ssp. | AA | Protein | TLLMSTTAAAVTKARENPYILALAANPTFVSGLVAWAVAQAAKVV | |
| | DUF212 | japonica | | | LTSFVERRWDLRMLFSSGGMPSSHTALCTALTASVALCHGVSDSLF | |
| | | | | | PVCLGFTLIVMYDATGVRRHAGMQAEV | |
| 80 | Os04g0486900 | Oryza sativa ssp. | AA | Protein | AAAVVNYPLVAALVAFALAQSSKFFTTWFKEKRWDARQLIASGG | |
| | DUF212 | japonica | | | MPSSHSATVTALAVAIGIQEGYRSATFATSVIIACVVMHDAFGVRL | |
| | | | | | HAGKQAEVLNQIVYELPEEHPLSETKPLREILGHTVPQVVAGCIIGILI | |
| | | | | | AVVMR | |
| 81 | Os01g0901800 | Oryza sativa ssp. | AA | Protein | SYFAVFHNYPLVAALLGFAVAQSIKFFVTRYKENRWDPKQLIGSGG | |
| | DUF212 | japonica | | | MPSSHSATVTALAVAIGFQDGFGCALFATAAIFASVVMYDASGIRL | |
| | | | | | HAGKQAEVLNQIVCELPSEHPLSETRPLRELLGHTPTQVVAGALLGS | |
| | | | | | MLATAGQM | |
| 82 | Os08g0127500 | Oryza sativa ssp. | AA | Protein | NCPLVAAVLAGAIAQFIKVLTTWYKENRWDAKQLVGSGGMPSSH | |
| | DUF212 | japonica | | | SATVVALAVAVGLQEGFGSSLFATAAIFASVVMYDAFGVRLHAGK | |
| 83 | Os05g0548800 | Oryza sativa ssp. | AA | Protein | QAEVLNQIVYELPSEHPLAETRPLRELLGHTPAQVFAGGVLGFAV | |
| | DUF212 | japonica | | | IFFLQVMYDASGIRFHTGRQAALLNQIVSDFPPEHPIISSFRPLQEPL | |
| | | | | | GHSPFQVFAGALVGCSIAYLMGK | |
| 84 | Os06g0530300 | Oryza sativa ssp. | AA | Protein | VAVATSLGLERGFADSIFGMSVVFAAIVMYDAQGVRREVGNHAR | |
| | DUF212 | japonica | | | VLNKLLTLREKITQNPDDNSLLSSTSELHSSKPETVAELVSVAEKLGSS | |
| | | | | | QGSSANPFPIHSSGTKSSSRLNALQSSETEVTEFTQLKEAYTEECDRLS | |
| | | | | | ESVGHTELQVAAGALLGFLVTLVVYA | |

1. A method for improving the tolerance of a plant or algae to salt, relative to a wild-type, comprising:
introducing a composition into one or more plant or algae cells, wherein the composition comprises a molecule that reduces the expression in the plant or algae cells of a nucleotide sequence encoding an amino acid sequence comprising at least a sequence 8590% identical to the sequences of the following list SEQ ID No 69, SEQ ID No 70, SEQ ID No 71, SEQ ID No 72, SEQ ID No 73, SEQ ID No 74, SEQ ID No 75, SEQ ID No 76, SEQ ID No 77, SEQ ID No 78, SEQ ID No 79, SEQ ID No 80, SEQ ID No 81, SEQ ID No 82, SEQ ID No 83, SEQ ID No 84, and wherein the molecule is a protein molecule, a nucleic acid molecule or combinations thereof; and
regenerating one or more plants or algae from said one or more plant or algae cells, wherein the regenerated plants or algae have increased salt tolerance relative to a wild-type plant or algae.

2. (canceled)

3. The method of claim 1, wherein the nucleotide sequence encodes an amino acid sequence comprising at least a sequence 90% identical to the sequences of the following list SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, or homologue thereof.

4. The method of claim 1, wherein the nucleotide sequence encodes an amino acid sequence comprising at least a sequence 95% identical to the sequences of the following list: SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, or a homologue thereof.

5. The method of claim 1, wherein the nucleotide sequence is a gene comprising at least a sequence 90% identical to the sequences of the following list SEQ ID No 16, SEQ ID No 17, SEQ ID No 18, SEQ ID No 19, SEQ ID No 20, or a homologue thereof.

6. The method of claim 5, wherein the gene comprises at least a sequence 95% identical to the sequences of the following list SEQ ID No 16, SEQ ID No 17, SEQ ID No 18, SEQ ID No 19, SEQ ID No 20, or homologue thereof.

7. (canceled)

8. (canceled)

9. (canceled)

10. The method of claim 1, wherein the nucleic acid molecule is selected from a T-DNA, a siRNA, a shRNA, a miRNA, a ribozyme, a peptide nucleic acid, sgRNA, or antisense oligonucleotide.

11. The method of claim 1, wherein the protein molecule is selected from a zinc-finger nuclease, transcription activator-like effector nuclease, or CRISPR-associated protein 9.

12. (canceled)

13. (canceled)

14. The method of claim 1, wherein the composition comprises a recombinant plant expression vector comprising the nucleic acid molecule or a nucleic acid that generates an RNA molecule encoding said protein molecule in plant cells; and
optionally a poly A signal sequence inducing polyadenylation at the 3′-end of the RNA molecule.

15. (canceled)

16. (canceled)

17. (canceled)

18. (canceled)

19. A method for producing a genetically modified plant or algae with increased salt tolerance relative to a wild-type plant, the method comprising the following steps:
introducing at least one mutation or exogenous nucleic acid into the genome of one or more plant or algae cells which results in reduced activity associated with a protein, wherein the protein comprising at least a sequence 90% identical to the sequences of the following list SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67, or a homologue thereof in said one or more plant or algae cells;
regenerating one or more plants or algae from said one or more plant or algae cells; and
selecting one or more plants or algae that have increased salt tolerance relative to a wild-type plant or algae.

20. The method of claim 19, wherein the method comprises introducing at least one mutation into a gene comprising at least a sequence 90% identical to the sequences of the following list SEQ ID No 16, SEQ ID No 17, SEQ ID No 18, SEQ ID No 19, SEQ ID No 20, or homologue thereof, or inhibiting or suppressing the expression of said gene or homologue thereof.

21. The method of claim 19, wherein the exogenous nucleic acid is a T-DNA.

22. (canceled)

23. The method of claim 19, wherein the exogenous nucleic acid comprises a nucleic acid complementary to at least a portion of the encoding sequence, or homologue thereof, of a protein comprising at least a sequence 90% identical to the sequences of the following list SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67.

24. The method of claim 19, further comprising a step of transforming a plant, plant tissue culture, or plant cell or algae with a vector comprising the exogenous nucleic acid.

25. The method of claim 24, wherein the vector is a binary vector, a virus derived vector, a plasmid, a liposome, a dendrimer, or nanoparticle vector.

26. The method of claim 24, wherein the vector comprises a sequence at least 90% identical to SEQ ID. No 68.

27. (canceled)

28. The method of claim 19, wherein the plant or algae is selected from Arabidopsis thaliana, Amborella trichopoda, Chlamydomonas reinhardtii, Medicago truncatula, Oryza sativa, Picea abies, Physcomitrium patens, Sequoiadendron giganteum, Selaginella moellendorffii, or Solanum lycopersicum.

29. A method for screening a plant or algae with increased salt tolerance relative to a wild-type plant, the method comprising analyzing DNA of the plant or algae for the presence of at least one allele of a nucleotide sequence encoding a protein comprising at least a sequence 90% identical to the sequences of the following list SEQ ID No 26, SEQ ID No 27, SEQ ID No 28, SEQ ID No 29, SEQ ID No 30, SEQ ID No 31, SEQ ID No 32, SEQ ID No 33, SEQ ID No 34, SEQ ID No 35, SEQ ID No 36, SEQ ID No 37, SEQ ID 38, SEQ ID No 39, SEQ ID No 40, SEQ ID No 41, SEQ ID No 42, SEQ ID No 43, SEQ ID No 44, SEQ ID No 45, SEQ ID No 46, SEQ ID No 47, SEQ ID No 48, SEQ ID No 49, SEQ ID No 50, SEQ ID No 51, SEQ ID No 52, SEQ ID No 53, SEQ ID No 54, SEQ ID No 55, SEQ ID No 56, SEQ ID No 57, SEQ ID No 58, SEQ ID No 59, SEQ ID No 60, SEQ ID No 61, SEQ ID No 62, SEQ ID No 63, SEQ ID No 64, SEQ ID No 65, SEQ ID No 66, SEQ ID No 67 or a homologue thereof, using at least one nucleic acid molecule suitable as a probe or primer which is capable of hybridizing to a gene comprising at least a sequence 90% identical to the sequences of the following list SEQ ID No 16, SEQ ID No 17, SEQ ID No 18, SEQ ID No 19, SEQ ID No 20, or a homologue thereof.

30. The method of claim 29, comprising using at least one oligonucleotide primer pair suitable for amplification of a region of a gene comprising at least a sequence 90% identical to the sequences of the following list SEQ ID No 16, SEQ ID No 17, SEQ ID No 18, SEQ ID No 19, SEQ ID No 20, or homologue thereof, said primer pair comprising a forward primer and a reverse primer to detect the presence or absence of a mutation in said region.

31. The method of claim 29, comprising the following steps:
obtaining a biological sample from a plant or algae;
contacting the sample with the at least one oligonucleotide primer pair;
performing a nucleic acid amplification reaction;
measuring the level of expression of a gene comprising at least a sequence 90% identical to the sequences of the following list: SEQ ID No 16, SEQ ID No 17, SEQ ID No 18, SEQ ID No 19, SEQ ID No 20, or homologue thereof;
comparing the level of expression of the gene in the biological sample with the level of expression of the gene in the wild-type sample;
wherein a lower level of expression of the gene corresponds to a higher tolerance to salt.

32. (canceled)

33. (canceled)

34. A plant or algae with increased salt tolerance relative to a wild-type plant or algae, obtained by the method of claim 19, wherein said plant is selected from Arabidopsis thaliana, Amborella trichopoda, Chlamydomonas reinhardtii, Medicago truncatula, Oryza sativa, Picea abies, Physcomitrium patens, Sequoiadendron giganteum, Selaginella moellendorffii, or Solanum lycopersicum, with the proviso that said plant or algae is not an Arabidopsis thaliana SALK_030394, or SALK_039758 mutant.

35. (canceled)

36. (canceled)
